SEQUENCE LISTING (24 sequences)

SEQ ID NO: 1 - DNA, 1626 bp, Homo sapiens
atgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttcctcgagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttaa

SEQ ID NO: 2 - protein, 541 aa, Homo sapiens
MNCRELPLTLWVLISVSTAESCTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADMADIPGHVFTRGMIIAVLILVAVVCLVTVCVIYRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 3 - DNA, 1800 bp, Homo sapiens
atgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaaggagcaaccgaatcgtagtggatgaagtttatgactatcaccagggcacatatgtatgtgattacactcagtcggatactgtgagttcgtggacagtcagagctgttgttcaagtgagaaccattgtgggagacactaaactcaaaccagatattctggatcctgtcgaggacacactggaagtagaacttggaaagcctttaactattagctgcaaagcacgatttggctttgaaagggtctttaaccctgtcataaaatggtacatcaaagattctgacctagagtgggaagtctcagtacctgaggcgaaaagtattaaatccactttaaaggatgaaatcattgagcgtaatatcatcttggaaaaagtcactcagcgtgatcttcgcaggaagtttgtttgctttgtccagaactccattggaaacacaacccagtccgtccaactgaaagaaaagagaggagtggtgctcctgtacatcctgcttggcaccatcgggaccctggtggccgtgctggcggcgagtgccctcctctacaggcactggattgaaatagtgctgctgtaccggacctaccagagcaaggatcagacgcttggggataaaaaggattttgatgctttcgtatcctatgcaaaatggagctcttttccaagtgaggccacttcatctctgagtgaagaacacttggccctgagcctatttcctgatgttttagaaaacaaatatggatatagcctgtgtttgcttgaaagagatgtggctccaggaggagtgtatgcagaagacattgtgagcattattaagagaagcagaagaggaatatttatcttgagccccaactatgtcaatggacccagtatctttgaactacaagcagcagtgaatcttgccttggatgatcaaacactgaaactcattttaattaagttctgttacttccaagagccagagtctctacctcatctcgtgaaaaaagctctcagggttttgcccacagttacttggagaggcttaaaatcagttcctcccaattctaggttctgggccaaaatgcgctaccacatgcctgtgaaaaactctcagggattcacgtggaaccagctcagaattacctctaggatttttcagtggaaaggactcagtagaacagaaaccactgggaggagctcccagcctaaggaatggtga

SEQ ID NO: 4 - protein, 599 aa, Homo sapiens
MLCLGWIFLWLVAGERIKGFNISGCSTKKLLWTYSTRSEEEFVLFCDLPEPQKSHFCHRNRLSPKQVPEHLPFMGSNDLSDVQWYQQPSNGDPLEDIRKSYPHIIQDKCTLHFLTPGVNNSGSYICRPKMIKSPYDVACCVKMILEVKPQTNASCEYSASHKQDLLLGSTGSISCPSLSCQSDAQSPAVTWYKNGKLLSVERSNRIVVDEVYDYHQGTYVCDYTQSDTVSSWTVRAVVQVRTIVGDTKLKPDILDPVEDTLEVELGKPLTISCKARFGFERVFNPVIKWYIKDSDLEWEVSVPEAKSIKSTLKDEIIERNIILEKVTQRDLRRKFVCFVQNSIGNTTQSVQLKEKRGVVLLYILLGTIGTLVAVLAASALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEW

SEQ ID NO: 5 - DNA, 1713 bp, Homo sapiens
atgacacttctgtggtgtgtagtgagtctctacttttatggaatcctgcaaagtgatgcctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaagtgccagctccaagatacacagtggaactggcttgtggttttggagccacagtcctgctagtggtgattctcattgttgtttaccatgtttactggctagagatggtcctattttaccgggctcattttggaacagatgaaaccattttagatggaaaagagtatgatatttatgtatcctatgcaaggaatgcggaagaagaagaatttgtattactgaccctccgtggagttttggagaatgaatttggatacaagctgtgcatctttgaccgagacagtctgcctgggggaattgtcacagatgagactttgagcttcattcagaaaagcagacgcctcctggttgttctaagccccaactacgtgctccagggaacccaagccctcctggagctcaaggctggcctagaaaatatggcctctcggggcaacatcaacgtcattttagtacagtacaaagctgtgaaggaaacgaaggtgaaagagctgaagagggctaagacggtgctcacggtcattaaatggaaaggggaaaaatccaagtatccacagggcaggttctggaagcagctgcaggtggccatgccagtgaagaaaagtcccaggcggtctagcagtgatgagcagggcctctcgtattcatctttgaaaaatgtatga

SEQ ID NO: 6 - protein, 570 aa, Homo sapiens
MTLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVELACGFGATVLLVVILIVVYHVYWLEMVLFYRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNV

SEQ ID NO: 7 - DNA, 1728 bp, Homo sapiens
atgtggtccttgctgctctgcgggttgtccatcgcccttccactgtctgtcacagcagatggatgcaaggacatttttatgaaaaatgagatactttcagcaagccagccttttgcttttaattgtacattccctcccataacatctggggaagtcagtgtaacatggtataaaaattctagcaaaatcccagtgtccaaaatcatacagtctagaattcaccaggacgagacttggattttgtttctccccatggaatggggggactcaggagtctaccaatgtgttataaagggtagagacagctgtcatagaatacatgtaaacctaactgtttttgaaaaacattggtgtgacacttccataggtggtttaccaaatttatcagatgagtacaagcaaatattacatcttggaaaagatgatagtctcacatgtcatctgcacttcccgaagagttgtgttttgggtccaataaagtggtataaggactgtaacgagattaaaggggagcggttcactgttttggaaaccaggcttttggtgagcaatgtctcggcagaggacagagggaactacgcgtgtcaagccatactgacacactcagggaagcagtacgaggttttaaatggcatcactgtgagcattacagaaagagctggatatggaggaagtgtccctaaaatcatttatccaaaaaatcattcaattgaagtacagcttggtaccactctgattgtggactgcaatgtaacagacaccaaggataatacaaatctacgatgctggagagtcaataacactttggtggatgattactatgatgaatccaaacgaatcagagaaggggtggaaacccatgtctcttttcgggaacataatttgtacacagtaaacatcaccttcttggaagtgaaaatggaagattatggccttcctttcatgtgccacgctggagtgtccacagcatacattatattacagctcccagctccggattttcgagcttacttgataggagggcttatcgccttggtggctgtggctgtgtctgttgtgtacatatacaacatttttaagatcgacattgttctttggtatcgaagtgccttccattctacagagaccatagtagatgggaagctgtatgacgcctatgtcttataccccaagccccacaaggaaagccagaggcatgccgtggatgccctggtgttgaatatcctgcccgaggtgttggagagacaatgtggatataagttgtttatattcggcagagatgaattccctggacaagccgtggccaatgtcatcgatgaaaacgttaagctgtgcaggaggctgattgtcattgtggtccccgaatcgctgggctttggcctgttgaagaacctgtcagaagaacaaatcgcggtctacagtgccctgatccaggacgggatgaaggttattctcattgagctggagaaaatcgaggactacacagtcatgccagagtcaattcagtacatcaaacagaagcatggtgccatccggtggcatggggacttcacggagcagtcacagtgtatgaagaccaagttttggaagacagtgagataccacatgccgcccagaaggtgtcggccgtttcctccggtccagctgctgcagcacacaccttgctaccgcaccgcaggcccagaactaggctcaagaagaaagaagtgtactctcacgactggctaa

SEQ ID NO: 8 - protein, 575 aa, Homo sapiens
MWSLLLCGLSIALPLSVTADGCKDIFMKNEILSASQPFAFNCTFPPITSGEVSVTWYKNSSKIPVSKIIQSRIHQDETWILFLPMEWGDSGVYQCVIKGRDSCHRIHVNLTVFEKHWCDTSIGGLPNLSDEYKQILHLGKDDSLTCHLHFPKSCVLGPIKWYKDCNEIKGERFTVLETRLLVSNVSAEDRGNYACQAILTHSGKQYEVLNGITVSITERAGYGGSVPKIIYPKNHSIEVQLGTTLIVDCNVTDTKDNTNLRCWRVNNTLVDDYYDESKRIREGVETHVSFREHNLYTVNITFLEVKMEDYGLPFMCHAGVSTAYIILQLPAPDFRAYLIGGLIALVAVAVSVVYIYNIFKIDIVLWYRSAFHSTETIVDGKLYDAYVLYPKPHKESQRHAVDALVLNILPEVLERQCGYKLFIFGRDEFPGQAVANVIDENVKLCRRLIVIVVPESLGFGLLKNLSEEQIAVYSALIQDGMKVILIELEKIEDYTVMPESIQYIKQKHGAIRWHGDFTEQSQCMKTKFWKTVRYHMPPRRCRPFPPVQLLQHTPCYRTAGPELGSRRKKCTLTTG

SEQ ID NO: 9 - DNA, 1671 bp, Homo sapiens
atggggttttggatcttagcaattctcacaattctcatgtattccacagcagcaaagtttagtaaacaatcatggggcctggaaaatgaggctttaattgtaagatgtcctagacaaggaaaacctagttacaccgtggattggtattactcacaaacaaacaaaagtattcccactcaggaaagaaatcgtgtgtttgcctcaggccaacttctgaagtttctaccagctgcagttgctgattctggtatttatacctgtattgtcagaagtcccacattcaataggactggatatgcgaatgtcaccatatataaaaaacaatcagattgcaatgttccagattatttgatgtattcaacagtatctggatcagaaaaaaattccaaaatttattgtcctaccattgacctctacaactggacagcacctcttgagtggtttaagaattgtcaggctcttcaaggatcaaggtacagggcgcacaagtcatttttggtcattgataatgtgatgactgaggacgcaggtgattacacctgtaaatttatacacaatgaaaatggagccaattatagtgtgacggcgaccaggtccttcacggtcaaggatgagcaaggcttttctctgtttccagtaatcggagcccctgcacaaaatgaaataaaggaagtggaaattggaaaaaacgcaaacctaacttgctctgcttgttttggaaaaggcactcagttcttggctgccgtcctgtggcagcttaatggaacaaaaattacagactttggtgaaccaagaattcaacaagaggaagggcaaaatcaaagtttcagcaatgggctggcttgtctagacatggttttaagaatagctgacgtgaaggaagaggatttattgctgcagtacgactgtctggccctgaatttgcatggcttgagaaggcacaccgtaagactaagtaggaaaaatccaattgatcatcatagcatctactgcataattgcagtatgtagtgtatttttaatgctaatcaatgtcctggttatcatcctaaaaatgttctggattgaggccactctgctctggagagacatagctaaaccttacaagactaggaatgatggaaagctctatgatgcttatgttgtctacccacggaactacaaatccagtacagatggggccagtcgtgtagagcactttgttcaccagattctgcctgatgttcttgaaaataaatgtggctataccttatgcatttatgggagagatatgctacctggagaagatgtagtcactgcagtggaaaccaacatacgaaagagcaggcggcacattttcatcctgacccctcagatcactcacaataaggagtttgcctacgagcaggaggttgccctgcactgtgccctcatccagaacgacgccaaggtgatacttattgagatggaggctctgagcgagctggacatgctgcaggctgaggcgcttcaggactccctccagcatcttatgaaagtacaggggaccatcaagtggagggaggaccacattgccaataaaaggtccctgaattctaaattctggaagcacgtgaggtaccaaatgcctgtgccaagcaaaattcccagaaaggcctctagtttgactcccttggctgcccagaagcaatag

SEQ ID NO: 10 - protein, 556 aa, Homo sapiens
MGFWILAILTILMYSTAAKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPIDHHSIYCIIAVCSVFLMLINVLVIILKMFWIEATLLWRDIAKPYKTRNDGKLYDAYVVYPRNYKSSTDGASRVEHFVHQILPDVLENKCGYTLCIYGRDMLPGEDVVTAVETNIRKSRRHIFILTPQITHNKEFAYEQEVALHCALIQNDAKVILIEMEALSELDMLQAEALQDSLQHLMKVQGTIKWREDHIANKRSLNSKFWKHVRYQMPVPSKIPRKASSLTPLAAQKQ

SEQ ID NO: 11 - protein, 21 aa, Artificial
  MOG35-55
MEVGWYRSPFSRVVHLYRNGK

SEQ ID NO: 12 - protein, 17 aa, Artificial
  p11 peptide
GPDIYKGVYQFKSVEFD

SEQ ID NO: 13 - protein, 3 aa, Artificial
  linker
EFM

SEQ ID NO: 14 - protein, 13 aa, Artificial
  Linker
EFGAGLVLGGQFM

SEQ ID NO: 15 - protein, 15 aa, Artificial
  Linker
GGGGSGGGGSGGGGS

SEQ ID NO: 16 - protein, 16 aa, Artificial
  Linker
GGSGGSGGGGSGGGGS

SEQ ID NO: 17 - DNA, 1710 bp, Homo sapiens
atgaaagtgttactcagacttatttgtttcatagctctactgatttcttctctggaggctgataaatgcaaggaacgtgaagaaaaaataattttagtgtcatctgcaaatgaaattgatgttcgtccctgtcctcttaacccaaatgaacacaaaggcactataacttggtataaagatgacagcaagacacctgtatctacagaacaagcctccaggattcatcaacacaaagagaaactttggtttgttcctgctaaggtggaggattcaggacattactattgcgtggtaagaaattcatcttactgcctcagaattaaaataagtgcaaaatttgtggagaatgagcctaacttatgttataatgcacaagccatatttaagcagaaactacccgttgcaggagacggaggacttgtgtgcccttatatggagttttttaaaaatgaaaataatgagttacctaaattacagtggtataaggattgcaaacctctacttcttgacaatatacactttagtggagtcaaagataggctcatcgtgatgaatgtggctgaaaagcatagagggaactatacttgtcatgcatcctacacatacttgggcaagcaatatcctattacccgggtaatagaatttattactctagaggaaaacaaacccacaaggcctgtgattgtgagcccagctaatgagacaatggaagtagacttgggatcccagatacaattgatctgtaatgtcaccggccagttgagtgacattgcttactggaagtggaatgggtcagtaattgatgaagatgacccagtgctaggggaagactattacagtgtggaaaatcctgcaaacaaaagaaggagtaccctcatcacagtgcttaatatatcggaaattgaaagtagattttataaacatccatttacctgttttgccaagaatacacatggtatagatgcagcatatatccagttaatatatccagtcactaatttccagaagcacatgattggtatatgtgtcacgttgacagtcataattgtgtgttctgttttcatctataaaatcttcaagattgacattgtgctttggtacagggattcctgctatgattttctcccaataaaagcttcagatggaaagacctatgacgcatatatactgtatccaaagactgttggggaagggtctacctctgactgtgatatttttgtgtttaaagtcttgcctgaggtcttggaaaaacagtgtggatataagctgttcatttatggaagggatgactacgttggggaagacattgttgaggtcattaatgaaaacgtaaagaaaagcagaagactgattatcattttagtcagagaaacatcaggcttcagctggctgggtggttcatctgaagagcaaatagccatgtataatgctcttgttcaggatggaattaaagttgtcctgcttgagctggagaaaatccaagactatgagaaaatgccagaatcgattaaattcattaagcagaaacatggggctatccgctggtcaggggactttacacagggaccacagtctgcaaagacaaggttctggaagaatgtcaggtaccacatgccagtccagcgacggtcaccttcatctaaacaccagttactgtcaccagccactaaggagaaactgcaaagagaggctcacgtgcctctcgggtag

SEQ ID NO: 18 - protein, 569 aa, Homo sapiens
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNFQKHMIGICVTLTVIIVCSVFIYKIFKIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 19 - DNA, 12183 bp, Artificial
  pCEP4 (ECD mouse IL-18Ra-hIgG1 CHC)
ctagctccaccatgggatggtcatgtatcatcctttttctagtagcaactgcaaccggtagttgtattcaccgatcacaaattcatgtggtagagggagaacctttttatctgaagccatgtggcatatctgcaccagtgcacaggaatgaaacagccaccatgagatggttcaaaggcagtgcttcacatgagtatagagagctgaacaacagaagctcgcccagagtcacttttcatgatcacaccttggaattctggccagttgagatggaggatgagggaacgtacatttctcaagtcggaaatgatcgtcgcaattggaccttaaatgtcaccaaaagaaacaaacacagctgtttctctgacaagctcgtgacaagcagagatgttgaagttaacaaatctctgcatatcacttgtaagaatcctaactatgaagagctgatccaggacacatggctgtataagaactgtaaggaaatatccaaaaccccaaggatcctgaaggatgccgagtttggagatgagggctactactcctgcgtgttttctgtccaccataatgggacacggtacaacatcaccaagactgtcaatataacagttattgaaggaaggagtaaagtaactccagctattttaggaccaaagtgtgagaaggttggtgtagaactaggaaaggatgtggagttgaactgcagtgcttcattgaataaagacgatctgttttattggagcatcaggaaagaggacagctcagaccctaatgtgcaagaagacaggaaggagacgacaacatggatttctgaaggcaaactgcatgcttcaaaaatactgagatttcagaaaattactgaaaactatctcaatgttttatataattgcaccgtggccaacgaagaagccatagacaccaagagcttcgtcttggtgagaaaagaaatacctgatatcccaggccatgtctttacaggaggaggcggtggctcgggcggtggtgggtcgggtggcggcggatcccggtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagctggcacccaacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgagcggccgctcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccagctg

SEQ ID NO: 20 - DNA, 11631 bp, Artificial
  pCEP4 (ECD mouse IL-18Rb-hK CLC)
ctagctccaccatgggatggtcatgtatcatcctttttctagtagcaactgcaaccggtgagaagaccacaggatttaatcattcagcttgtgccaccaaaaaacttctgtggacatattctgcaaggggtgcagagaattttgtcctattttgtgacttacaagagcttcaggagcaaaaattctcccatgcaagtcaactgtcaccaacacaaagtcctgctcacaaaccttgcagtggcagtcagaaggacctatctgatgtccagtggtacatgcaacctcggagtggaagtccactagaggagatcagtagaaactctccccatatgcagagtgaaggcatgctgcatatattggccccacagacgaacagcatttggtcatatatttgtagacccagaattaggagcccccaggatatggcctgttgtatcaagacagtcttagaagttaagcctcagagaaacgtgtcctgtgggaacacagcacaagatgaacaagtcctacttcttggcagtactggctccattcattgtcccagtctcagctgccaaagtgatgtacagagtccagagatgacctggtacaaggatggaagactacttcctgagcacaagaaaaatccaattgagatggcagatatttatgtttttaatcaaggcttgtatgtatgtgattacacacagtcagataatgtgagttcctggacagtccgagctgtggttaaagtgagaaccattggtaaggacatcaatgtgaagccggaaattctggatcccattacagatacactggacgtagagcttggaaagcctttaactctcccctgcagagtacagtttggcttccaaagactttcaaagcctgtgataaagtggtatgtcaaagaatctacacaggagtgggaaatgtcagtatttgaggagaaaagaattcaatccactttcaagaatgaagtcattgaacgtaccatcttcttgagagaagttacccagagagatctcagcagaaagtttgtttgctttgcccagaactccattgggaacacaacacggaccatacggctgaggaagaaggaagagggaggcggtggctcgggcggtggtgggtcgggtggcggcggatccctcgagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggcggccgctcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccagctg

SEQ ID NO: 21 - DNA, 11622 bp, Artificial
  pCEP4 (ECD mouse IL-1RacP-hK CLC)
ccggttcggagcgctgtgatgactggggactagataccatgcgacaaatccaagtgtttgaagatgagccggctcgaatcaagtgccccctctttgaacacttcctgaagtacaactacagcactgcccattcctctggccttaccctgatctggtactggaccaggcaagaccgggacctggaggagcccattaacttccgcctcccagagaatcgcatcagtaaggagaaagatgtgctctggttccggcccaccctcctcaatgacacgggcaattacacctgcatgttgaggaacacaacttactgcagcaaagttgcatttcccctggaagttgttcagaaggacagctgtttcaattctgccatgagattcccagtgcacaagatgtatattgaacatggcattcataagatcacatgtccaaatgtagacggatactttccttccagtgtcaaaccatcggtcacttggtataagggttgtactgaaatagtggactttcataatgtactacccgagggcatgaacttgagctttttcatccccttggtttcaaataacggaaattacacatgtgtggttacatatcctgaaaacggacgtctctttcacctcaccaggactgtgactgtaaaggtggtgggctcaccaaaggatgcattgccaccccagatctattctccaaatgaccgtgttgtctatgagaaagaaccaggagaggaactggttattccctgcaaagtctatttcagtttcattatggactcccacaatgaggtctggtggaccattgatggaaagaagcctgatgacgtcacagtcgacatcactattaatgaaagtgtaagttattcttcaacggaagatgaaacaaggactcagattttgagcatcaagaaagtcaccccggaggatctcaggcgcaactatgtctgtcatgctcgaaataccaaaggggaagctgagcaggctgccaaggtgaaacagaaagtcataccaccaaggtacacagtagaaggaggcggtggctcgggcggtggtgggtcgggtggcggcggatccctcgagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggcggccgctcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccagctgctagctccaccatgggatggtcatgtatcatcctttttctagtagcaactgcaa

SEQ ID NO: 22 - DNA, 11565 bp, Artificial
  pCEP4 (ECD mouse IL-1Rrp2-hK CLC)
ccggtgcagatacgtgtgaggacatttttatgcacaatgtgataatttcagagggccagccttttcctttcaactgcacatacccgccagaaacaaacggggcagtaaatctgacatggtacaaaacacctagcaaaagcccagtatctaacaacagacaccttagagttcaccaggaccagacctggatcttgtttcttccattgacactggaggactccggtatctatcagtgtgttataaggaatgcccacaactgctaccaaatagctgtgaacctaaccgttttaaaaaaccactggtgtgactcttccatggaggggagtcccgtaaattcaccagatgtgtaccagcaaatattacccataggaaaatcgggcagtctgaattgtcatctctacttcccagaaagttgtgctttggattcaataaaatggtataagggttgtgaagagattaaagcggggaaaaagtacagcccttcaggagcaaagcttcttgtgaacaacgttgctgtggaggacggcgggagctatgcgtgctcagccagactgactcacttggggagacacttcaccattagaaactacattgctgtgaacaccaaggaagttgagtatggaagaaggatccctaacatcacgtatccaaagaacaactccattgaagttccacttggctccaccctcatcgtgaactgcaatataacagacacgaaggagaatacaaacctgaggtgctggagagtcaacaacaccctggtggatgactactacaaagactccaaacgcatccaggaaggaatcgaaaccaatgtgtccttgagggatcaaattcggtacacagtgaacataacattcttaaaagtgaaaatggaggactacggccgtcctttcacgtgtcatgctggagtgtccgcagcctacatcattctgatatacccagttccagacttcagggcttacggaggcggtggctcgggcggtggtgggtcgggtggcggcggatccctcgagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggcggccgctcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccagctgctagctccaccatgggatggtcatgtatcatcctttttctagtagcaactgcaa

SEQ ID NO: 23 - DNA, 11526 bp, Artificial
  pCEP4 (ECD mouse T1/ST2-hK CLC)
ccggtagtaaatcgtcctggggtctggaaaatgaggctttaattgtgagatgcccccaaagaggacgctcgacttatcctgtggaatggtattactcagatacaaatgaaagtattcctactcaaaaaagaaatcggatctttgtctcaagagatcgtctgaagtttctaccagccagagtggaagactctgggatttatgcttgtgttatcagaagccccaacttgaataagactggatacttgaatgtcaccatacataaaaagccgccaagctgcaatatccctgattatttgatgtactcgacagtacgtggatcagataaaaatttcaagataacgtgtccaacaattgacctgtataattggacagcacctgttcagtggtttaagaactgcaaagctctccaagagccaaggttcagggcacacaggtcctacttgttcattgacaacgtgactcatgatgatgaaggtgactacacttgtcaattcacacacgcggagaatggaaccaactacatcgtgacggccaccagatcattcacagttgaagaaaaaggcttttctatgtttccagtaattacaaatcctccatacaaccacacaatggaagtggaaataggaaaaccagcaagtattgcctgttcagcttgctttggcaaaggctctcacttcttggctgatgtcctgtggcagattaacaaaacagtagttggaaattttggtgaagcaagaattcaagaagaggaaggtcgaaatgaaagttccagcaatgacatggattgtttaacctcagtgttaaggataactggtgtgacagaaaaggacctgtccctggaatatgactgtctggccctgaaccttcatggcatgataaggcacaccataaggctgagaaggaaacaaccaattgatcaccgaagcggaggcggtggctcgggcggtggtgggtcgggtggcggcggatccctcgagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggcggccgctcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccagctgctagctccaccatgggatggtcatgtatcatcctttttctagtagcaactgcaa

SEQ ID NO: 24 - DNA, 11565 bp, Artificial
  pCEP4 (ECD mouse IL-1R1-hK CLC)
ccggtgagattgacgtatgtacagaatatccaaatcagatcgttttgtttttatctgtaaatgaaattgatattcgcaagtgtcctcttactccaaataaaatgcacggcgacaccataatttggtacaagaatgacagcaagacccccatatcagcggaccgggactccaggattcatcagcagaatgaacatctttggtttgtacctgccaaggtggaggactcaggatattactattgtatagtaagaaactcaacttactgcctcaaaactaaagtaaccgtaactgtgttagagaatgaccctggcttgtgttacagcacacaggccaccttcccacagcggctccacattgccggggatggaagtcttgtgtgcccttatgtgagttattttaaagatgaaaataatgagttacccgaggtccagtggtataagaactgtaaacctctgcttcttgacaacgtgagcttcttcggagtaaaagataaactgttggtgaggaatgtggctgaagagcacagaggggactatatatgccgtatgtcctatacgttccgggggaagcaatatccggtcacacgagtaatacaatttatcacaatagatgaaaacaagagggacagacctgttatcctgagccctcggaatgagacgatcgaagctgacccaggatcaatgatacaactgatctgcaacgtcacgggccagttctcagaccttgtctactggaagtggaatggatcagaaattgaatggaatgatccatttctagctgaagactatcaatttgtggaacatccttcaaccaaaagaaaatacacactcattacaacacttaacatttcagaagttaaaagccagttttatcgctatccgtttatctgtgttgttaagaacacaaatatttttgagtcggcgcatgtgcagttaatatacccagtccctgacttcaagaattacggaggcggtggctcgggcggtggtgggtcgggtggcggcggatccctcgagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggcggccgctcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccagctgctagctccaccatgggatggtcatgtatcatcctttttctagtagcaactgcaa

(19) United States
(12) Patent Application Publication
(10) Pub. No.: US 2008/0292590 A1
(43) Pub. Date: Nov. 27, 2008

(54) Soluble Receptors and Methods for Treating Autoimmune or Demyelinating Diseases

(76) Inventors: Burkhard Becher, Maur (CH); Elisabeth Saller, Zurich (CH)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 12/159,421

(22) PCT Filed: Feb. 21, 2007
(86) PCT No.: PCT/EP2007/051695

§ 371 Date: Jun. 27, 2008

Related U.S. Application Data
(60) Provisional application No. 60/792,841, filed on Apr. 18, 2006.

(30) Foreign Application Priority Data
Feb. 22, 2006 (EP) 06110304.0

(51) Int. Cl. A61K 38/21; C07K 2/00; A61P 37/02; A61K 38/02

(52) U.S. Cl. 424/85.6; 530/350; 514/12

(57) ABSTRACT

The present invention relates to novel therapeutic protein useful in the treatment of diseases, in particular in human subjects. The results of the inventor strongly support the use of soluble IL-18Rα in the treatment of diseases such as autoimmune or demyelinating disease, in particular Multiple Sclerosis (MS). Accordingly, the invention provides soluble IL-18Rα for use in the treatment of autoimmune or demyelinating disease, in particular MS. The invention also provides methods of treating, preventing or ameliorating the symptoms of autoimmune or demyelinating disease, in particular MS, in a subject, preferably a human subject, by administering a therapeutically effective amount of said soluble IL-18Rα to the subject.

The present invention relates to novel therapeutic protein useful in the treatment of diseases, in particular in human subjects.

As explained herein, the results of the inventor strongly support the use of soluble IL-18Rα in the treatment of diseases such as autoimmune or demyelinating disease, in particular Multiple Sclerosis (MS). Accordingly, the invention provides soluble IL-18Rα for use in the treatment of autoimmune or demyelinating disease, in particular MS. The invention also provides methods of treating, preventing or ameliorating the symptoms of autoimmune or demyelinating disease, in particular MS, in a human subject, by administering a therapeutically effective amount of said soluble IL-18Rα to the subject.

### BACKGROUND

Demyelinating diseases are a group of pathologies that involve abnormalities in myelin sheaths of the nervous system. Many congenital metabolic disorders affect the developing myelin sheath, mainly in the CNS, and demyelination is a feature of many neurological disorders.

The most known chronic inflammatory demyelinating disease of the central nervous system in humans is multiple sclerosis. The onset of multiple sclerosis (MS) typically occurs during ages 20 to 40. Women are affected approximately twice as often as men. Over time, MS may result in the accumulation of various neurological disabilities. Clinical disability in MS is presumed to be a result of repeated inflammatory injury with subsequent loss of myelin and axons, leading to tissue atrophy.

MS is manifested in physical symptoms (relapses and disability progression), central nervous system (CNS) inflammation, brain atrophy and cognitive impairment. Presenting symptoms include focal sensory deficits, focal weakness, visual problems, imbalance and fatigue. Sexual impairment and sphincter dysfunction may occur. Approximately half of the patients with MS may experience cognitive impairment or depression.

MS is now considered to be a multi-phasic disease, and periods of clinical quiescence (remissions) occur between exacerbations. Remissions vary in length and may last several years but are infrequently permanent.

Four courses of the disease are individualized: relapsing-remitting (RR), secondary progressive (SP), primary progressive (PP) and progressive relapsing (PR) multiple sclerosis. More than 80% of patients with MS initially display a RR course with clinical exacerbation of neurological symptoms, followed by a recovery that may or may not be complete (Lublin and Reingold, Neurology, 1996, 46:907-911).

During RRMS, accumulation of disability results from incomplete recovery from relapses. Approximately, half of the patients with RRMS switch to a progressive course, called SPMS, 10 years after the diseased onset. During the SP phase, worsening of disability results from the accumulation of residual symptoms after exacerbation but also from insidious progression between exacerbations (Lublin and Reingold above). 10% of MS patients have PPMS which is characterized by insidious progression of the symptoms from the disease onset. Less than 5% of patients have PRMS and are often considered to have the same prognosis as PPMS. It is suggested that distinct pathogenic mechanisms may be involved in different patient sub-groups and have wide-ranging implications for disease classification (Lassmann et al., 2001, Trends Mol. Med., 7, 115-121; Lucchinetti et al., Curr. Opin. Neurol., 2001, 14, 259-269).

MS onset is defined by the occurrence of the first neurological symptoms of CNS dysfunction. Advances in cerebrospinal fluid (CSF) analysis and magnetic resonance imaging (MRI) have simplified the diagnostic process and facilitated early diagnostic (Noseworthy et al., The New England Journal of Medicine, 2000, 343, 13, 938-952). The International Panel on the Diagnosis of MS issued revised criteria facilitating the diagnosis of MS and including MRI together with clinical and para-clinical diagnostic methods (Mc Donald et al., 2001, Ann. Neurol., 50:121-127).

Treatments currently available for the treatment of multiple sclerosis essentially act against the symptoms of the disease. Consequently, there is a strong need for alternative therapies that provide improved clinical benefits to patients.

### SUMMARY OF THE PRESENT INVENTION

The present invention relates to novel therapeutic or prophylactic treatment in human subjects. The results disclosed herein strongly support the use of soluble IL-18Rα in the treatment of diseases, such as autoimmune or demyelinating disease, in particular Multiple Sclerosis (MS). Accordingly, the invention provides soluble IL-18Rα for use in the treatment of autoimmune or demyelinating disease, in particular MS. The invention also provides methods of treating, preventing or ameliorating the symptoms of an autoimmune or demyelinating disease, in particular MS, in a human subject by administering a therapeutically effective amount of said soluble IL-18Rα to the subject.

In a particular aspect, the invention resides in a soluble receptor comprising all or part of the extracellular domain of IL-18Rα, in particular comprising all or part of the extracellular domain of human IL-18Rα or a variant thereof.

In a further aspect, the invention resides in the soluble receptor as defined above comprising amino acids residues 19-132 of SEQ ID NO: 2, and/or amino acids residues 122-219 of SEQ ID NO: 2, and/or amino acids residues 213-329 of SEQ ID NO: 2, and/or a variant of said amino acid residues.

In a further aspect, the invention resides in the soluble receptor as defined above comprising amino acids residues 19-219 of SEQ ID NO: 2, and/or amino acids residues 122-329 of SEQ ID NO: 2, and/or amino acids residues 19-132 and 213-329 of SEQ ID NO:2 linked by a peptide bond, and/or a variant of said amino acid residues.

In a further aspect, the invention resides in the soluble receptor as defined above comprising amino acids residues 19-329 of SEQ ID NO: 2, and/or a variant of said amino acid residues.

In a further aspect, the invention resides in the soluble receptor as defined above wherein said variant of said amino acid residues is a polypeptide having at least 80% identity with said amino acid residues.

The invention further relates to the soluble receptor as defined above comprising at least two subunits consisting of amino acids residues 19-132 of SEQ ID NO: 2, and/or amino acids residues 122-219 of SEQ ID NO: 2, and/or amino acids residues 213-329 of SEQ ID NO: 2, and/or amino acids residues 19-219 of SEQ ID NO: 2, and/or 122-329 of SEQ ID NO: 2, and/or amino acids residues 19-132 and 213-329 of SEQ ID NO:2 linked by a peptide bond, and/or amino acids residues 19-329 of SEQ ID NO: 2, and/or a variant of said amino acid residues, on the same protein backbone as a fusion protein. In a particular embodiment, said variant of said amino acid residues is a polypeptide having at least 80% identity with said amino acid residues. In another particular embodiment, at least two subunits are the same.

The invention further relates to the soluble receptor as defined above operably linked to an additional amino acid domain.

In a further aspect, the invention resides in a multimer, in particular a dimer of a soluble receptor as defined above.

In a further aspect, the invention resides in a soluble receptor as defined above comprising in addition at least one IL-18Rβ subunit comprising all or part of the extracellular domain of IL-18Rβ, or at least one IL-1RacP subunit comprising all or part of the extracellular domain of IL-1RacP, or at least one IL-1R-rp2 subunit comprising all or part of the extracellular domain of IL-1R-rp2, or at least one T1/ST2 subunit comprising all or part of the extracellular domain of T1/ST2, or at least one IL-1R-1 subunit comprising all or part of the extracellular domain of IL-1R-1.

In a further aspect, the invention resides in a soluble receptor as defined above for use as a medicament.

The invention further relates to the use of a soluble receptor as defined above in the manufacture of a medicament for the treatment of an autoimmune or demyelinating disease. In particular embodiment, said demyelinating disease is multiple sclerosis.

In a further aspect, the invention resides in a method of treating, preventing or ameliorating the symptoms of an autoimmune or demyelinating disease in a subject, in particular a human subject, said method comprising administering to the subject a therapeutically effective amount of a soluble receptor as defined above. In particular embodiment, said demyelinating disease is multiple sclerosis.

The invention further relates to the method or use as defined above wherein the subject is affected by relapsing-remitting (RR) multiple sclerosis, secondary progressive (SP) multiple sclerosis, primary progressive (PP) multiple sclerosis or progressive relapsing (PR) multiple sclerosis.

The invention further relates to the method or use as defined above wherein the soluble receptor is administered in conjunction with a second therapeutic agent for treating or preventing MS. In a particular embodiment, the soluble receptor is administered in conjunction with corticosteroïds, immunosuppressive drugs, neuro-protective agents, immunomodulatory drugs or interferons. In yet another particular embodiment, the soluble receptor is administered in conjunction with interferon-beta, preferably with interferon beta-1a, even more preferably with Rebif® (Serono).

The invention further relates to a product comprising a soluble receptor as defined above and a corticosteroïd, an immunosuppressive drug, a neuro-protective agent, an immunomodulatory drug or an interferon as a combined preparation for simultaneous, separate or sequential use in the therapy of MS in a mammalian subject, preferably a human subject. In a particular embodiment, the interferon is interferon-beta, preferably interferon beta-1a, even more preferably Rebif® (Serono).

### LEGEND TO THE FIGURES

FIG. 1: IL-18R signaling, independent of IL-18, is required for EAE induction. Mice were actively immunized with MOG₃₅₋₅₅ in CFA and injected with pertussis toxin i.p. on days 0 and 2. (a) EAE progression in p35_{-/-}xIL-18_{-/-} double knockout and wt mice. Shown is one representative of 2 experiments (n=5 mice/group).

(b) EAE progression in wt. IL-18_{-/-} and IL-18Rα_{-/-} mice. Shown is one representative of 3 experiments (n=5 mice/group).

FIG. 2: IL-18R signaling, independent of IL-18, is required for EAE induction. Mice were actively immunized with MOG₃₅₋₅₅ in CFA and injected with pertussis toxin i.p. on days 0 and 2. (a) H&E, (b) LFB, (c) CD3, (d) MAC3 and (e) B220 stainings of PFA-fixed spinal cords from wt (score 2), IL-18−/− (score 2), IL-18Rα−/− (score 0) EAE mice and a naïve mouse showing infiltration relative to disease score.

FIG. 3: IL-18−/− LN cells do not produce IL-18 in agreement with their proposed genotype. ELISA assessing IL-18 secretion by naïve wt and IL-18−/− LN cells, stimulated for 16 hours with the indicated mixes of 1 μg/ml LPS, 100 Units/ml IFNγ, 5 μg/ml Concanavalin A (ConA) and 2.5 ng/ml IL-12.

FIG. 4: IL-18 and IL-18Rα are required for mitogen-stimulated T cell activation but not for Th1 development. (a) ELISA assessing IFNγ secretion by naïve wt. IL-18_{-/-} and IL-18Rα_{-/-} LN cells, stimulated for 16 hours with 5 μg/ml Concanavalin A (ConA).

(b,c) Mice were immunized with 200 μg KLH and 7 days later LN were isolated and restimulated.
(b) ELISA of IFNγ in supernatant from KLH immunized mice restimulated in duplicate with 50 μg/ml KLH or 5 μg/ml ConA for 48 hours.
(c) Proliferation assay of LN cells from KLH immunized mice restimulated in triplicate with 50 μg/ml KLH, 5 μg/ml ConA or medium for 48 hours. ³H-thymidine was added to the culture 24 hours prior to measuring proliferation in counts per minute (CPM).
(d) BM-derived DC's were generated from wt. IL-18_{-/-} and IL18R_{-/-} mice, matured with LPS and subsequently pulsed with 1 μg/ml SMARTA peptide, p11. p11-specific CD4₊ T cells were obtained from naïve SMARTA-Tg mice and cocultured with the peptide-pulsed, irradiated (2000 rads) DC's for 72 h when proliferation was assessed by thymidine incorporation in counts per minute (CPM).

FIG. 5: An alternative IL-18Rα-binding ligand induces EAE in IL-18_{-/-} mice. (a) IL-18_{-/-} were treated with 450 μg anti-IL-18Rα antibody (white square) or control IgG (black rhomb) 1 day pre-immunization with MOG₃₅₋₅₅ and with 300 μg antibody for every 3 days thereafter. Shown is one representative of 2 experiments (n=5 mice/group).

(b) IL-18_{-/-} mice (n=6 mice/group) were immunized with MOG₃₅₋₅₅ and treated with 300 μg anti-IL-18Rα antibody (white square) or control IgG (black rhomb) at the first sign of disease.

FIG. 6: IL-18R−/− CD4+ T cells are activated similar to wt and IL-18−/− CD4+ T cells. FACS of splenocytes derived from KLH immunized wt. IL-18−/− and IL-18R−/− mice, restimulated in vitro for 2 days with 50 μg/ml KLH or medium. After 2 days, spleen cells were stained with CD4-FITC and (a) CD5-APC, (b) CD62L-bio-SA-PerCP-Cy5.5 or (c) CD44-PE.

FIG. 7: IL-18Rα_{-/-}CD4₊ T cells infiltrate the CNS to the same extent as wt and IL-18_{-/-} CD4₊ T cells prior to disease onset. wt, IL-18_{-/-} and IL-18Rα_{-/-} mice were actively immunized with MOG₃₅₋₅₅ and on day 7 post-immunization the mice were perfused with PBS and the CNS was isolated. A gradient was performed to isolate microglia cells and the infiltration of inflammatory cells in this portion was assessed by flow cytometry. Cells were stained with CD45-PerCP and CD4-APC. IL-18Rα_{-/-}CD4₊ T cells invade the CNS and do so to the same as wt and IL-18_{-/-}CD4₊ T cells on day 7 post-immunization.

FIG. 8: The IL-18Rα lesion affects the production of IL-17 and the development of T_{H}IL-17 cells. Wt. IL-18_{-/-} and IL-18Rα_{-/-} mice were immunized with KLH and 7 days later, splenocytes were isolated and restimulated with 50 μg/ml KLH. (a) Real-time PCR comparison of IL-17 mRNA expression by wt. IL-18_{-/-} and IL-18Rα_{-/-} lymphocytes after 2 days in vitro restimulation with KLH. Results are normalized to β-actin expression and analyzed in duplicate. (b) ELISA of IL-17 protein expression by lymphocytes restimulated for 2 days with KLH in vitro in duplicate. Data combine at least 2 mice per group.

FIG. 9: The absence of IL-18Rα does not lesion T cells or B cells. BM-chimeric mice were generated by transferring 12-25×10⁶ BM-cells into lethally irradiated wt mice. 6 weeks later, reconstituted IL-18Rα_{-/-}→wt (grey triangle), IL-18Rα_{-/-}+RAG_{-/-}→wt (white square) and wt→wt (black rhomb) bone-marrow chimeric mice were actively immunized with MOG₃₅₋₅₅ peptide and clinical score was assessed. The presence of IL-18Rα on non-T and -B cells derived from the RAG_{-/-} bone marrow rescued the susceptibility of IL-18Rα_{-/-}→wt mice to EAE.

FIG. 10: IL-18Rα_{-/-} mice are resistant to the passive transfer of EAE. MOG-reactive lymphocytes were generated by actively immunizing wt mice, isolating spleen and LN cells after 11 days and restimulating them for 4 days in vitro with 20 μg/ml MOG₃₅₋₅₅ and 2.5 ng/ml IL-12. EAE was induced in recipient mice by the adoptive transfer of 20-30×10⁶ MOG-reactive lymphocytes into IL-18Rα_{-/-} (grey triangle) and wt (black rhomb) mice. Shown is one representative of 2 experiments (n=5 mice/group).

FIG. 11: Anti-IL-18Rα Ab treatment does not alter the composition of peripheral immune cells. IL-18_{-/-} mice were treated with 300 μg anti-IL-18Rα antibody or control IgG 1 day pre-immunization with MOG₃₅₋₅₅. 7 days later, spleens were isolated, homogenized and immune cell composition was assessed by flow cytometry. Cells were stained for CD8-FITC, CD4-APC, NK1.1-bio-SA-PerCP and B220-PE or CD11b-FITC, CD11c-APC and GR1-bio-SA-PerCP. There is no difference in immune cell composition in anti-IL-18Rα Ab-treated IL-18−/− mice. Shown is one representative FACS of 2 mice/group.

FIG. 12: Interfering activity of the recombinant antibody (catcher αβ) with IL-18 signaling in vitro. Wild type mouse splenocytes were tested for IFNγ secretion after stimulation with the indicated cytokines and antibodies. AB is a commercially available monoclonal anti-IL-18Rα antibody (clone 112624) (R&D Systems), rat IgG is an isotypic control antibody and catcher αβ.

### DETAILED DESCRIPTION OF THE INVENTION

As explained herein, the results of the inventor strongly support the use of soluble IL-18Rα in the treatment of diseases, such as autoimmune or demyelinating disease, in particular Multiple Sclerosis (MS). Accordingly, the invention provides soluble IL-18Rα for use in the treatment of autoimmune or demyelinating disease, in particular MS. The invention also provides methods of treating, preventing or ameliorating the symptoms of an autoimmune or demyelinating disease, in particular MS, in a human subject, by administering a therapeutically effective amount of said soluble IL-18Rα to the subject.

IL-18 Receptor has been described as a heterodimer consisting of a ligand-binding IL-18Rα-subunit (also named IL-1Rrp or IL-1R5 in the literature) and a signaling IL-18Rβ-subunit. Downstream signaling of the IL-18R, like that of the TLR pathway, activates IRAK4 and MyD88. IL-18Rα is expressed on lymphocytes and has more recently been found to be expressed on accessory cells (Kaser, A. et al. Blood 103, 648-655 (2004), Tomura, M. et al. Immunol. 160, 3759-3765 (1998), Xu, D. et al. J. Exp. Med. 188, 1485-1492 (1998), Yoshimoto, T. et al. J. Immunol. 161, 3400-3407 (1998)).

While it is established that IL-18 can bind to the IL-18R complex, its affinity to IL-18Rα alone is only weak (Boraschi, D. et al. Eur. Cytokine Netw. 9, 205-212 (1998), Torigoe, K. et al. J. Biol. Chem. 272, 25737-25742 (1997)). IL-18 collaborates with IL-12 to stimulate the production of IFN-γ by T cells and can independently stimulate the cytotoxic activity of NK cells. IL-18 and IL-12 act synergistically to polarize T cells towards a T_{H}1 cytokine response, which was thought to be a prerequisite for encephalitogenicity.

IL-18_{-/-} mice have been described as being EAE resistant and insufficient NK-cell activation in IL-18_{-/-} mice was thought to be the cause for the inability to generate an encephalitogenic immune response (Shi, F. D., et al., J. Immunol. 165, 3099-3104 (2000)). Nevertheless, the proposed role of IL-18 in EAE causes a dilemma given the clearly protective activity of IL-12 (Cua, D. J. et al. Nature 421, 744-748 (2003), Becher, B., et al., J. Clin. Invest 110, 493-497 (2002)).

The inventor now demonstrates that, in contrast to the previously published data, IL-18 does not exert a visible pathogenic effect in EAE as deduced by the susceptibility of IL-18_{-/-} mice to EAE. However, deletion of its proposed receptor (IL-18Rα) results in complete resistance to EAE induction, suggesting the presence of an alternative ligand (IL-18RL) with encephalitogenic properties. As the affinity of IL-18 to IL-18Rα is fairly poor and requires heterotrimerization with IL-18Rβ for increased affinity, the possibility that there is another ligand with higher affinity for IL-18Rα is very strong. There are a number of orphan receptors within the IL-1R superfamily and given the fact that these receptor subunits form heterodimers with one another, it is most likely that the IL-18Rα not only has different binding partners, but also different ligands.

The inventor demonstrates here the potency of this putative ligand by significantly attenuating disease development in IL-18_{-/-} mice using anti-IL-18Rα antibodies. Given that the accepted IL-18Rα-ligand, IL-18, was not present in these mice and that their cellular constituents were not affected as a result of injecting the antibody, these results provide substantial evidence for the existence of such an alternative IL-18Rα ligand.

Despite the importance of T cells during EAE, the inventor shows here that deletion of IL-18Rα does not affect T cell priming with regards to expansion and Th1 polarization. Alternatively, IL-18 and IL-18Rα are both required for efficient T cell activation when stimulated with the mitogen ConA, which concurs with the finding that IL-18_{-/-} mice have a defect in stimulating IFNγ secretion, as observed in various bacterial and viral infectious models. In agreement with a lack of disturbance at the level of T cell activation, the inventor shows here that the IL-18Rα lesion does not affect the activatory functions of Antigen presenting cells (APCs) as TcR Tg T cells proliferated to the same extent when cultured with wt (wild type), IL-18_{-/-} or IL-18Rα_{-/-} Dendritic Cells (DCs).

In contrast to the absence of inflammatory cells in the CNS at the endpoint of EAE the inventor could detect comparable CD4₊ T cell infiltration in the IL-18Rα_{-/-} CNS prior to the onset of disease. Other inflammatory cells also infiltrated the CNS to the same extent as in wt and IL-18_{-/-} mice. Therefore the IL-18Rα deficiency does not affect invasion of immune cells into the CNS but must affect their ability to persist. Interestingly, the presence of inflammatory infiltrates in the IL-18Rα_{-/-} CNS, without concomitant EAE susceptibility, resembles the response that occurs in IL-23_{-/-} mice.

The inventor analyzes IL-17 production by IL-18Rα_{-/-} KLH recall lymphocytes and demonstrates that there is indeed a significant decrease in the production of IL-17 at both the RNA and protein levels. Therefore the resistance of IL-18Rα_{-/-} mice to EAE could be explained as a result of insufficient T_{H}IL-17 development.

It seemed likely that the lack of T_{H}IL-17 cells resulted from the absence of IL-18Rα expression on this subpopulation of T cells. This was not the case, however, as the generation of BM-chimeras demonstrated that only in the presence of RAG_{-/-} BM cells could the susceptibility of IL-18Rα_{-/-} mice (RAG_{-/-}+IL-18Rα^{-/-}>wt) to EAE be rescued. IL-18Rα_{-/-}>wt mice, on the other hand, were resistant to disease induction. Therefore, the presence of IL-18Rα is required on a non-lymphocytic leukocyte and is not directly located on pre-T_{H}IL-17 cells. Furthermore, the importance of IL-18Rα on an accessory cell was accentuated in an adoptive transfer experiment whereby encephalitogenic wt T cells could not induce EAE in IL-18Rα_{-/-} mice.

In summary, the inventor shows evidence refuting the T_{H}1 hypothesis of MS and EAE by demonstrating a non-pathogenic role for IL-18 in EAE. In contrast, however, the so-called IL-18Rα is critical for the development of EAE thus implying the presence of an alternative IL-18Rα-binding ligand, which the inventor could confirm by treating IL-18_{-/-} mice with anti-IL-18Rα antibodies thereby diminishing EAE severity. Alternatively, the inventor show that IL-18Rα signaling is critical for the development of encephalitogenic T_{H}IL-17 cells, which thereby explains the resistance of IL-18Rα_{-/-} mice to MOG₃₅₋₅₅-induced EAE.

As explain herein, the inventor of the present invention has discovered that antagonists of IL-18Rα are effective in vivo for treating diseases. Moreover, the IL-18Rα antagonist also inhibited the progression of an already established disease, in a mouse model of MS.

Basis, in part, for the invention are the results disclosed here above and in the examples of the present application. These results strongly support the use of soluble IL-18Rα in the treatment of autoimmune or demyelinating disease, in particular MS. Accordingly, the invention provides soluble IL-18Rα for use in the treatment of autoimmune or demyelinating disease, in particular MS. The invention also provides methods of treating, preventing or ameliorating the symptoms of an autoimmune or demyelinating disease, in particular MS, in a human subject by administering a therapeutically effective amount of said soluble IL-18Rα to the subject.

As used herein, a “therapeutically effective amount” of a compound means the minimum amount of the compound that is effective to treat, ameliorate or prevent an autoimmune or demyelinating disease, in particular MS or its symptoms. The invention also pertains to the use of said soluble IL-18Rα in the manufacture of a medicament for the treatment of autoimmune or demyelinating disease, in particular MS.

In some embodiments of the present invention, the disease to treat is relapsing-remitting (RR) MS, secondary progressive (SP) MS, primary progressive (PP) MS or progressive relapsing (PR) MS.

As explain herein, the inventor of the present invention has discovered that antagonists of IL-18Rα are effective in vivo for treating diseases. The data obtained by the inventor strongly support that inhibition of IL-18Rα is effective for treating autoimmune or demyelinating disease, in particular MS, in an IL-18 independent manner. Therefore, in an embodiment of the present invention, the soluble IL-18Rα of the present invention used to treat the autoimmune or demyelinating disease, in particular MS, do not inhibit solely IL-18 activity. IL-18 Binding Protein (IL-18BP, which is described in PCT Publication WO 99/09063) is not considered as a soluble IL-18Rα according to the present invention.

The invention also pertains to any of the above or below described soluble IL-18Rα for use as a medicament.

In a specific embodiment of the invention, the soluble IL-18Rα of the present invention are capable of inhibiting the activity of IL18Rα in Antigen presenting cells and more specifically in the Antigen presenting cells selected from the group consisting of monomorphonucleated phagocytes, polymorphonucleated phagocytes, dendritic cells and Natural Killer cells.

In an embodiment of the invention, the soluble IL-18Rα of the present invention are capable of inhibiting the development of IL-17 producing TH cells.

A cDNA encoding human IL-18Rα is presented at SEQ ID NO: 1. This cDNA encodes a 541 amino acids long protein (SEQ ID NO: 2) which includes an extracellular domain of 329 amino acids (residues 1-329 of SEQ ID NO: 2) that includes a signal peptide of 18 amino acids (residues 1-18 of SEQ ID NO: 2), a transmembrane region of 21 amino acids (residues 330 to 350 of SEQ ID NO: 2), and, a cytoplasmic domain from amino acids 351 to 541 of SEQ ID NO: 2.

### 1) Soluble IL-18Rα:

Soluble IL-18Rα of the present invention are soluble receptors comprising all or part of the extracellular domain of IL-18Rα. In particular soluble receptors of the present invention are soluble receptors comprising all or part of the extracellular domain of human IL-18Rα or a variant thereof. Such soluble receptors are used to treat, prevent or ameliorate the symptoms of an autoimmune or demyelinating disease, in particular MS, in a subject, preferably a human subject.

A “soluble receptor” is a receptor polypeptide that is not bound to a cell membrane. Soluble receptors are most commonly receptor polypeptides that lack part or all of the transmembrane domains, and other linkage to the cell membrane such as via glycophosphoinositol (gpi) that would cause retention of the polypeptide at the cell surface. Soluble receptors may include part of the transmembrane domain and/or all or part of the cytoplasmic domain as long as the polypeptide is secreted from the cell in which it is produced. Soluble receptors can comprise additional amino acid residues, such as affinity tags that provide for purification of the polypeptide or provide sites for attachment of the polypeptide to a substrate, or immunoglobulin constant region sequences, as will be described here after. Soluble IL-18Rα receptors advantageously comprise a native or heterologous signal peptide when initially synthesized, to promote secretion from the cell, but the signal sequence is cleaved upon secretion.

IL-18Rα is a member of the so-called IL-1R family and possess an extracellular domain comprising three immunoglobulin-like domains (Ig domains).

IL-18Rα Subunit and Variants Thereof (Named here after “Sol(IL-18Rα)”):

In one aspect, the soluble receptor of the present invention (Sol(IL-18Rα)) is a soluble IL-18Rα comprising or consisting of amino acids residues 19-329 of SEQ ID NO: 2, or a variant of said polypeptide. Ordinarily, the variant polypeptides are at least 275 amino acids in length, often at least 300 amino acids in length, more often at least 311 amino acids in length. A variant is defined as a polypeptide having at least 80% amino acid sequence identity with the sequence of reference (here residues 19-329 of SEQ ID NO: 2), preferably at least 90% amino acid sequence identity, more preferably at least 95% amino acid sequence identity, more preferably at least 98% amino acid sequence identity and most preferably at least 99% amino acid sequence identity. More preferably, the variants are differing from the sequence of reference (here residues 19-329 of SEQ ID NO: 2) by five, more preferably by four, even more preferably by three, even more preferably by two and most preferably by one amino acid. In some particular aspects of the invention, the variants are differing from the sequence of reference (here residues 19-329 of SEQ ID NO: 2) by the lack of 20, 15, 10, 9, 8, 7, 6, 5, 4, 3, 2 or 1 amino acid(s) at the N-terminal and/or C-terminal end. One of skill in the art using the genetic code can readily determine polynucleotides that encode such polypeptides. “Percent (%) amino acid sequence identity” with respect to the polypeptide sequence of reference, is defined as the percentage of amino acid residues in a candidate sequence that are identical with the amino acid residues in the sequence of reference, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST (Altschul S F, Gish W, Miller W, Myers E W, Lipman D J. J Mol. Biol. (1990). 215 (3): 403-410). Those skilled in the art can determine appropriate parameters for measuring alignment, including any algorithms needed to achieve maximal alignment over the full length of the sequences being compared.

In another embodiment, Sol(IL-18Rα) is a polypeptide comprising or consisting of amino acids residues 19-219, or 122-329, or 19-132 and 213-329 linked by a peptide bond, of SEQ ID NO: 2, or a variant of said polypeptide. Ordinarily, the variant polypeptides are at least 180 amino acids in length, often at least 201 amino acids in length, often at least 208 amino acids in length, more often at least 231 amino acids in length. A variant is defined as a polypeptide having at least 80% amino acid sequence identity with the sequence of reference (here residues 19-219, or 122-329, or 19-132 and 213-329 linked by a peptide bond, of SEQ ID NO: 2), preferably at least 90% amino acid sequence identity, more preferably at least 95% amino acid sequence identity, more preferably at least 98% amino acid sequence identity and most preferably at least 99% amino acid sequence identity. More preferably, the variants are differing from the sequence of reference (here residues 19-219, or 122-329, or 19-132 and 213-329 linked by a peptide bond, of SEQ ID NO: 2), by five, more preferably by four, even more preferably by three, even more preferably by two and most preferably by one amino acid. In some particular aspects of the invention, the variants are differing from the sequence of reference (here residues 19-219, or 122-329, or 19-132 and 213-329 linked by a peptide bond, of SEQ ID NO: 2), by the lack of 20, 15, 10, 9, 8, 7, 6, 5, 4, 3, 2 or 1 amino acid(s) at the N-terminal and/or C-terminal end. One of skill in the art using the genetic code can readily determine polynucleotides that encode such polypeptides. “Percent (%) amino acid sequence identity” is defined as here above.

In yet another embodiment, Sol(IL-18Rα) is a polypeptide comprising or consisting of amino acids residues 19-132, or 122-219, or 213-329 of SEQ ID NO: 2, or a variant of said polypeptide. Ordinarily, the variant polypeptides are at least 90 amino acids in length, often at least 98 amino acids in length, often at least 114 amino acids in length, more often at least 117 amino acids in length. A variant is defined as a polypeptide having at least 80% amino acid sequence identity with the sequence of reference (here residues 19-132, or 122-219, or 213-329 of SEQ ID NO: 2), preferably at least 90% amino acid sequence identity, more preferably at least 95% amino acid sequence identity, more preferably at least 98% amino acid sequence identity and most preferably at least 99% amino acid sequence identity. More preferably, the variants are differing from the sequence of reference (here residues 19-132, or 122-219, or 213-329 of SEQ ID NO: 2) by five, more preferably by four, even more preferably by three, even more preferably by two and most preferably by one amino acid. In some particular aspects of the invention, the variants are differing from the sequence of reference (here residues 19-132, or 122-219, or 213-329 of SEQ ID NO: 2), by the lack of 20, 15, 10, 9, 8, 7, 6, 5, 4, 3, 2 or 1 amino acid(s) at the N-terminal and/or C-terminal end. One of skill in the art using the genetic code can readily determine polynucleotides that encode such polypeptides. “Percent (%) amino acid sequence identity” is defined as here above.

Soluble IL-18Rα Comprising at Least Two IL-18Rα Subunits or Variant thereof on the Same Protein Backbone (Named here after “Sol(IL-18Rα)ₓ”):

In a particular aspect of the present invention, the soluble IL-18Rα receptors of the present invention are soluble receptors comprising at least two IL-18Rα subunits, or variant thereof (i.e at least two Sol(IL-18Rα) subunits as defined here above) on the same protein backbone as a fusion protein. In a particular embodiment, the fusion protein comprises two Sol(IL-18Rα) subunits. In yet another particular embodiment, the at least two Sol(IL-18Rα) subunit are the same (i.e the fusion protein is a homomultimer of Sol(IL-18Rα)), and in a particular embodiment the fusion protein is a homodimer of Sol(IL-18Rα).

The at least two IL-18Rα subunit (Sol(IL-18Rα)) are operably linked to one another. The term “operably linked” indicates that the subunits are associated through peptide linkage, either directly or via a “peptide linker”. In this manner, the fusion protein can be produced recombinantly, by direct expression in a host cell of a nucleic acid molecule encoding the same. The subunits are linked either directly or via a “peptide linker”. The peptide linker can be as short as 1 to 3 amino acid residues in length (preferably consisting of small amino acids such as glycine, serine, threonine or alanine) or longer, for example 13, 15 or 16 amino acid residues in length, introduced between the subunits. Preferably, the peptide linker is a peptide which is immunologically inert. Said linker may be a tripeptide of the sequence E-F-M (Glu-Phe-Met) (SEQ ID NO: 13), for example, a 13-amino acid linker sequence consisting of Glu-Phe-Gly-Ala-Gly-Leu-Val-Leu-Gly-Gly-Gln-Phe-Met (SEQ ID NO: 14), a 15-amino acid linker sequence consisting of (G₄S)₃ (SEQ ID NO: 15), a 16-amino acid linker sequence consisting of GGSGG SGGGG SGGGG S (SEQ ID NO: 16) or the hinge region of human IgG (e.g. IgG1, IgG2, IgG3 or IgG4).

Soluble IL-18Rα (Sol(IL-18Rα) or Sol(IL-18Rα)ₓ) as Fusion Protein:

The soluble IL-18Rα receptors of the invention include fusion proteins. Accordingly, the present invention also relates to proteins comprising at least one IL-18Rα subunit or a variant thereof as described here above (Sol(IL-18Rα) or Sol(IL-18Rα)ₓ), operably linked to an additional amino acid domain. The additional amino acid domain may be located upstream (N-ter) or downstream (C-ter) from the sequence of Sol(IL-18Rα) or Sol(IL-18Rα)ₓ. The additional domain may comprise any functional region, providing for instance an increased stability, targeting or bioavailability of the fusion protein; facilitating purification or production, or conferring on the molecule additional biological activity. Specific examples of such additional amino acid sequences include a GST sequence, a His tag sequence, a multimerication domain, the constant region of an immunoglobulin molecule or a heterodimeric protein hormone such as human chorionic gonadotropin (hCG) as described in U.S. Pat. No. 6,193,972. The term “operably linked” indicates that Sol(IL-18Rα) or Sol(IL-18Rα)ₓ, and the additional amino acid domain are associated through peptide linkage, either directly or via a “peptide linker” (as defined here above). In this manner, the fusion protein can be produced recombinantly, by direct expression in a host cell of a nucleic acid molecule encoding the same. Also, if needed, the additional amino acid sequence included in the fusion proteins may be eliminated, either at the end of the production/purification process or in vivo, e.g., by means of an appropriate endo-/exopeptidase. For example, a spacer sequence included in the fusion protein may comprise a recognition site for an endopeptidase (such as a caspase) that can be used to separate by enzymatic cleavage the desired polypeptide variant from the additional amino acid domain, either in vivo or in vitro.

Multimers of Sol(IL-18Rα) and/or Sol(IL-18Rα)ₓ:

In a particular aspect of the present invention, Sol(IL-18Rα) and/or Sol(IL-18Rα)ₓ (as defined here above) are produced as multimers. Each subunit of the multimer comprising or consisting of Sol(IL-18Rα) and/or Sol(IL-18Rα)ₓ. These multimers may be homodimeric, heterodimeric, or multimeric soluble receptors, with multimeric receptors generally not comprising more than 9 subunits, preferably not comprising more than 6 subunits, even more preferably not more than 3 subunits and most preferably not comprising more than 2 subunits. Preferably, these multimers soluble receptors are homodimers of Sol(IL-18Rα) or Sol(IL-18Rα)ₓ. In an embodiment, the subunits of the multimers are linked via covalent linkages. The subunits may be covalently linked by any suitable means, such as via a cross-linking reagent or a polypeptide linker. In another embodiment, the subunits are linked via non-covalent linkages.

In one embodiment, the subunits are operably linked to an additional amino acid domain that provides for the multimerization of the subunits (in particular the additional domains comprise any functional region providing for dimerization of the subunits). The term “operably linked” indicates that Sol(IL-18Rα) or Sol(IL-18Rα)ₓ, and the additional amino acid domain are associated through peptide linkage, either directly or via a “peptide linker” (as defined here above). The additional amino acid domain may be located upstream (N-ter) or downstream (C-ter) from the sequence of Sol(IL-18Rα) or Sol(IL-18Rα)ₓ. In this manner, the fusion protein can be produced recombinantly, by direct expression in a host cell of a nucleic acid molecule encoding the same. In these embodiments, soluble IL-18Rα receptors of the invention are multimers of fusion proteins containing Sol(IL-18Rα) and/or Sol(IL-18Rα)ₓ components and a multimerizing component capable of interacting with the multimerizing component present in another fusion protein to form a higher order structure, such as a dimer. This type of fusion proteins may be prepared by operably linking the Sol(IL-18Rα) and/or Sol(IL-18Rα)ₓ sequence (as defined above) to domains isolated from other proteins allowing the formation of dimers, trimers, etc. Examples for protein sequences allowing the multimerization of the IL-18Rα soluble receptors of the invention are domains isolated from proteins such as immunoglobulins, hCG (WO 97/30161), collagen X (WO 04/33486), C4BP (WO 04/20639), Erb proteins (WO 98/02540), or coiled coil peptides (WO 01/00814).

In a particular aspect, the multimers are dimers of Sol(IL-18Rα) and/or Sol(IL-18Rα)ₓ where the subunits (Sol(IL-18Rα) and/or Sol(IL-18Rα)ₓ) are operably linked to an immunoglobulin. The term “operably linked” indicates that Sol(IL-18Rα) and/or Sol(IL-18Rα)ₓ, and the immunoglobulin are associated through peptide linkage, either directly or via a “peptide linker” (as defined here above). In this embodiment, the subunits are operably linked to all or a portion of an immunoglobulin, particularly a human immunoglobulin, even more particularly the Fc portion of a human immunoglobulin. Typically an Fc portion of a human immunoglobulin contains two constant region domains (the CH2 and CH3 domains) and a hinge region but lacks the variable region (See e.g. U.S. Pat. Nos. 6,018,026 and 5,750,375). The immunoglobulin may be selected from any of the major classes of immunoglobulins, including IgA, IgD, IgE, IgG and IgM, and any subclass or isotype, e.g. IgG1, IgG2, IgG3 and IgG4; IgA-1 and IgA-2. In an embodiment, the Fc moiety is of human IgG4, which is stable in solution and has little or no complement activating activity. In another embodiment, the Fc moiety is of human IgG1. The Fc part may be mutated in order to prevent unwanted activities, such as complement binding, binding to Fc receptors, or the like. Usually the subunits (Sol(IL-18Rα) and/or Sol(IL-18Rα)ₓ) are operably linked to the same immunoglobulin (particularly to the Fc portion of a human immunoglobulin, for example of a human IgG4 or human IgG1). The amino acid sequence derived from the immunoglobulin may be linked to the C-terminus or to the N-terminus of Sol(IL-18Rα) and/or Sol(IL-18Rα)ₓ, preferably to the C-terminus. Such fusion proteins can be prepared by transfecting cells with DNA encoding Sol(IL-18Rα):Fc fusion protein and/or DNA encoding Sol(IL-18Rα)ₓ:Fc fusion protein and expressing the dimers in the same cells. In a particular embodiment, the subunits Sol(IL-18Rα) or Sol(IL-18Rα)ₓ are the same on each monomer (i.e the dimer is a homodimer of Sol(IL-18Rα) or Sol(IL-18Rα)ₓ). Even more particularly, the subunits of Sol(IL-18Rα) or Sol(IL-18Rα)ₓ are operably linked to the same immunoglobulin (particularly to the Fc portion of a human immunoglobulin, for example of a human IgG4 or human IgG1). Such fusion proteins can be prepared by transfecting cells with DNA encoding Sol(IL-18Rα):Fc fusion protein or DNA encoding Sol(IL-18Rα)ₓ:Fc fusion protein and expressing the dimers in the same cells. Both subunits advantageously comprise a native or heterologous signal peptide when initially synthesized, to promote secretion from the cell, but the signal sequence is cleaved upon secretion. Methods for making immunoglobulin fusion proteins are well known in the art, such as the ones described in Hollenbaugh and Aruffo (“Construction of Immunoglobulin Fusion Proteins”, in Current Protocols in Immunology, Suppl. 4, pages 10.19.1-10.19.11, 1992) or WO 01/03737, for example.

Alternatively, the dimers of Sol(IL-18Rα) and/or Sol(IL-18Rα)ₓ of the present invention can be prepared by operably linking one of the receptor subunit to the constant region of an immunoglobulin heavy chain and operably linking the other receptor subunit to the constant region of an immunoglobulin light chain. The term “operably linked” indicates that Sol(IL-18Rα) and/or Sol(IL-18Rα)ₓ, and the immunoglobulin are associated through peptide linkage, either directly or via a “peptide linker” (as defined here above). For example, a Sol(IL-18Rα) or Sol(IL-18Rα)ₓ subunit can be operably linked to the CH₁-hinge-CH2-CH3 region of human IgG1 and another or the same Sol(IL-18Rα) or Sol(IL-18Rα)ₓ subunit can be operably linked to the C kappa region of the Ig kappa light chain. The amino acid sequence derived from the immunoglobulin may be linked to the C-terminus or to the N-terminus of the Sol(IL-18Rα) and/or Sol(IL-18Rα)ₓ subunits, preferably to the C-terminus. Cells transfected with DNA encoding the immunoglobulin light chain fusion protein and the immunoglobulin heavy chain fusion protein express heavy chain/light chain heterodimers containing each a Sol(IL-18Rα) or Sol(IL-18Rα)ₓ subunit. Both subunits advantageously comprise a native or heterologous signal peptide when initially synthesized, to promote secretion from the cell, but the signal sequence is cleaved upon secretion. In a particular embodiment, the subunits Sol(IL-18Rα) or Sol(IL-18Rα)ₓ are the same on each monomer (i.e the dimer is a homodimer of Sol(IL-18Rα) or Sol(IL-18Rα)ₓ).

In another particular aspect of the present invention, the subunits of the multimers Sol(IL-18Rα) and/or Sol(IL-18Rα)ₓ (as defined here above) are linked via non-covalent linkages. Non-covalent bonding of the subunits may be achieved by any suitable means that does not interfere with its biological activity (i.e. its ability to reduce the symptoms of MS). In a particular aspect, these multimers are dimers of Sol(IL-18Rα) and/or Sol(IL-18Rα)ₓ where one subunit of Sol(IL-18Rα) and/or Sol(IL-18Rα)ₓ is operably linked to a first compound and another or the same subunit Sol(IL-18Rα) and/or Sol(IL-18Rα)ₓ is operably linked to a second compound that will non-covalently bond to the first compound. The term “operably linked” is as defined here above. Examples of such compounds are biotin and avidin. The dimers of Sol(IL-18Rα) and/or Sol(IL-18Rα)ₓ can be prepared by operably linking one of the receptor subunit to biotin and operably linking the other receptor subunit to avidin. The receptor is thus formed through the non-covalent interactions of biotin with avidin. Other examples include subunits of heterodimeric proteinaceous hormone. In these embodiments, a DNA construct encoding one subunit of Sol(IL-18Rα) and/or Sol(IL-18Rα)ₓ is fused to a DNA construct encoding a subunit of a heterodimeric proteinaceous hormone, such as hCG, and a DNA construct encoding the other Sol(IL-18Rα) and/or Sol(IL-18Rα)ₓ subunit is fused to DNA encoding the other subunit of the heterodimeric proteinaceous hormone, such as hCG (as disclosed in U.S. Pat. No. 6,193,972). These DNA constructs are coexpressed in the same cells leading to the expression of an Sol(IL-18Rα) and/or Sol(IL-18Rα)ₓ heterodimeric receptor fusion protein, as each coexpressed sequence contains a corresponding hormone subunit so as to form a heterodimer upon expression. The amino acid sequence derived from the heterodimeric proteinaceous hormone may be linked to the C-terminus or to the N-terminus of the Sol(IL-18Rα) and/or Sol(IL-18Rα)ₓ subunits, preferably to the C-terminus. Both subunits advantageously comprise a native or heterologous signal peptide when initially synthesized, to promote secretion from the cell, but the signal sequence is cleaved upon secretion. In a particular embodiment, the subunits Sol(IL-18Rα) or Sol(IL-18Rα)ₓ are the same on each monomer (i.e the dimer is a homodimer of Sol(IL-18Rα) or Sol(IL-18Rα)ₓ).

Other examples for protein sequences allowing the dimerization of the Sol(IL-18Rα) and/or Sol(IL-18Rα)ₓ subunits are domains isolated from proteins such as collagen X (WO 04/33486), C4BP (WO 04/20639), Erb proteins (WO 98/02540), or coiled coil peptides (WO 01/00814).

In a particular aspect of the present invention, the multimer of Sol(IL-18Rα) and/or Sol(IL-18Rα)ₓ is a recombinant antibody. The technology of recombinant antibody is described for example in the U.S. Pat. No. 6,018,026. In that case, the multimer of Sol(IL-18Rα) and/or Sol(IL-18Rα)ₓ is a multimer polypeptide fusion, comprising: a first Sol(IL-18Rα) or Sol(IL-18Rα)ₓ polypeptide chain and a second Sol(IL-18Rα) and/or Sol(IL-18Rα)ₓ polypeptide chain, wherein the first polypeptide chain is operably linked to an immunoglobulin heavy chain constant region and the second polypeptide chain is operably linked to an immunoglobulin light chain constant region. The term “operably linked” indicates that Sol(IL-18Rα) and/or Sol(IL-18Rα)ₓ, and the immunoglobulin heavy or light chain constant region are associated through peptide linkage, either directly or via a “peptide linker” (as defined here above). In an embodiment, the immunoglobulin heavy chain constant region domain and the immunoglobulin light chain constant region domain are human immunoglobulin constant regions. In an embodiment, the immunoglobulin heavy chain constant region domain is selected from the group consisting of the constant region of an α, γ, μ, δ or ε human immunoglobulin heavy chain. Preferably, said constant region is the constant region of a γ1, γ2, γ3 or γ4 human immunoglobulin heavy chain. In a preferred embodiment, the immunoglobulin light chain constant region domain is selected from the group consisting of the constant region of a κ or λ human immunoglobulin light chain. The amino acid sequence from the immunoglobulin may be linked to the C-terminus or to the N-terminus of the Sol(IL-18Rα) and/or Sol(IL-18Rα)ₓ subunits, preferably to the C-terminus. Cells transfected with DNA encoding the immunoglobulin light chain fusion protein and the immunoglobulin heavy chain fusion protein express a fusion protein having the structure of an antibody: a protein consisting of two identical heavy chains constant region operably linked to a Sol(IL-18Rα) or Sol(IL-18Rα)ₓ subunit and two identical light chains constant region operably linked to a Sol(IL-18Rα) or Sol(IL-18Rα)ₓ. As for an antibody, heavy and light chains are disulfide linked (interchain disulfide bond) and heavy chains are disulfide linked (interchain disulfide bond). The resulting molecule is therefore an homodimer composed of two heterodimers each of these heterodimers being composed of:

an immunoglobulin heavy chain constant region operably linked to a first Sol(IL-18Rα) or Sol(IL-18Rα)ₓ polypeptide chain and;

an immunoglobulin light chain constant region operably linked to a second Sol(IL-18Rα) or Sol(IL-18Rα)ₓ polypeptide chain.

Both subunits advantageously comprise a native or heterologous signal peptide when initially synthesized, to promote secretion from the cell, but the signal sequence is cleaved upon secretion. In a particular embodiment, the subunits Sol(IL-18Rα) or Sol(IL-18Rα)ₓ are the same on the light and the heavy chains (i.e the recombinant antibody is composed of four Sol(IL-18Rα) or Sol(IL-18Rα)ₓ subunits that are the same).

In an embodiment, the heavy constant chain is human γ4, which is stable in solution and has little or no complement activating activity. In another embodiment, the heavy constant chain is human γ1. The heavy constant chain may be mutated in order to prevent unwanted activities, such as complement binding, binding to Fc receptors, or the like.

1. In an embodiment the recombinant antibody of the present invention comprises or consists of:

two identical heavy chains constant regions, said heavy chains constant regions being the constant region of γ1, γ2, γ3 or γ4 human immunoglobulin heavy chain, operably linked to the extracellular domain of the human IL-18Rα and;

two identical light chains constant regions, said light chain constant regions being the constant region of κ or λ human immunoglobulin light chain, operably linked to the extra cellular domain of the human IL-18Rα. In an embodiment, heavy and light chains are disulfide linked (interchain disulfide bond) and heavy chains are disulfide linked (interchain disulfide bond) as for a natural antibody.

2. In another embodiment, the present invention resides in a recombinant antibody as defined at point 1 above wherein the constant regions of the heavy chain are the constant regions of γ1 human immunoglobulin heavy chain.

3. In another embodiment, the present invention resides in a recombinant antibody as defined at point 1 or 2 above wherein the constant regions of the light chain are the constant regions of κ human immunoglobulin light chain.

4. In another embodiment, the present invention resides in a recombinant antibody as defined at point 1, 2 or 3 above wherein the extra cellular domain of the human IL-18Rα operably linked to the heavy chain consists of amino acids residues 19-329 of SEQ ID NO: 2 or a variant of said polypeptide as defined here above.

5. In another embodiment, the present invention resides in a recombinant antibody as defined at point 1, 2, 3 or 4 above wherein the extra cellular domain of the human IL-18Rα operably linked to the light chain consists of amino acids residues 19-329 of SEQ ID NO: 2 or a variant of said polypeptide as defined here above.

6. In another embodiment, the present invention resides in a recombinant antibody as defined at point 1, 2, 3, 4 or 5 above wherein the heavy chain constant regions are directly associated through peptide linkage to the extracellular domain of the human IL-18Rα.

7. In another embodiment, the present invention resides in a recombinant antibody as defined at point 1, 2, 3, 4, 5 or 6 above wherein the light chain constant regions are directly associated through peptide linkage to the extracellular domain of the human IL-18Rα.

8. In another embodiment, the present invention resides in a recombinant antibody as defined at point 1, 2, 3, 4 or 5 above wherein the heavy chain constant regions are associated through peptide linkage via a peptide linker to the extracellular domain of the human IL-18Rα. The peptide linker can be as short as 1 to 3 amino acid residues in length (preferably consisting of small amino acids such as glycine, serine, threonine or alanine) or longer, for example 13, 15 or 16 amino acid residues in length, introduced between the subunits. Preferably, the peptide linker is a peptide which is immunologically inert. Said linker may be a tripeptide of the sequence E-F-M (Glu-Phe-Met) (SEQ ID NO: 13), for example, a 13-amino acid linker sequence consisting of Glu-Phe-Gly-Ala-Gly-Leu-Val-Leu-Gly-Gly-Gln-Phe-Met (SEQ ID NO: 14), a 15-amino acid linker sequence consisting of (G₄S)₃ (SEQ ID NO: 15), a 16-amino acid linker sequence consisting of GGSGG SGGGG SGGGG S (SEQ ID NO: 16) or the hinge region of human IgG (e.g. IgG1, IgG2, IgG3 or IgG4). In an embodiment, said peptide linker is a 15-amino acid linker sequence consisting of (G₄S)₃ (SEQ ID NO: 15),

9. In another embodiment, the present invention resides in a recombinant antibody as defined at point 1, 2, 3, 4, 5, or 8 above wherein the light chain constant regions are associated through peptide linkage via a peptide linker to the extracellular domain of the human IL-18Rα. The peptide linker can be as short as 1 to 3 amino acid residues in length (preferably consisting of small amino acids such as glycine, serine, threonine or alanine) or longer, for example 13, 15 or 16 amino acid residues in length, introduced between the subunits. Preferably, the peptide linker is a peptide which is immunologically inert. Said linker may be a tripeptide of the sequence E-F-M (Glu-Phe-Met) (SEQ ID NO: 13), for example, a 13-amino acid linker sequence consisting of Glu-Phe-Gly-Ala-Gly-Leu-Val-Leu-Gly-Gly-Gln-Phe-Met (SEQ ID NO: 14), a 15-amino acid linker sequence consisting of (G₄S)₃ (SEQ ID NO: 15), a 16-amino acid linker sequence consisting of GGSGG SGGGG SGGGG S (SEQ ID NO: 16) or the hinge region of human IgG (e.g. IgG1, IgG2, IgG3 or IgG4). In an embodiment, said peptide linker is a 15-amino acid linker sequence consisting of (G₄S)₃ (SEQ ID NO: 15).

10. In another embodiment, the present invention resides in a recombinant antibody as defined at point 1, 2, 3, 4, 5, 6, 7, 8 or 9 above wherein the heavy constant chain is human γ4, which is stable in solution and has little or no complement activating activity.

11. In another embodiment, the present invention resides in a recombinant antibody as defined at point 1, 2, 3, 4, 5, 6, 7, 8 or 9 above wherein the heavy constant chain is human γ1 and is mutated in order to prevent unwanted activities, such as complement binding, binding to Fc receptors, or the like.

12. In another embodiment, the present invention resides in a recombinant antibody as defined at point 1, 2, 3, 4, 5, 6, 7, 8, 9, 10 or 11 above wherein the heavy chain constant regions are operably linked to the C-terminus or to the N-terminus of the extracellular domain of the human IL-18Rα, preferably to the C-terminus.

13. In another embodiment, the present invention resides in a recombinant antibody as defined at point 1, 2, 3, 4, 5, 6, 7, 8, 9, 10, 11 or 12 above wherein the light chain constant regions are operably linked to the C-terminus or to the N-terminus of the extracellular domain of the human IL-18Rα, preferably to the C-terminus.

14. In another embodiment, the present invention resides in a recombinant antibody as defined at point 1, 2, 3, 4, 5, 6, 7, 8, 9, 10, 11, 12 or 13 above wherein the extracellular domain of the human IL-18Rα is operably linked to the C-terminus or to the N-terminus of the heavy chain constant regions, preferably to the N-terminus.

15. In another embodiment, the present invention resides in a recombinant antibody as defined at point 1, 2, 3, 4, 5, 6, 7, 8, 9, 10, 11, 12, 13 or 14 above wherein the extracellular domain of the human IL-18Rα is operably linked to the C-terminus or to the N-terminus of the light chain constant regions, preferably to the N-terminus.

Also, if needed, fusion proteins described herein may comprise any functional region facilitating purification or production. Specific examples of such additional amino acid sequences include a GST sequence or a His tag sequence.

2) Soluble IL-18Rα Comprising at Least One IL-18Rα Subunit (Sol(IL-18Rα) and/or Sol(IL-18Rα)ₓ) and one IL-18Rβ subunit (Sol(IL-18Rβ) and/or Sol(IL-18Rβ)ₓ):

In a particular aspect of the present invention, the soluble IL-18Rα receptors used to treat, prevent or ameliorate the symptoms of an autoimmune or demyelinating disease, in particular MS, are soluble receptors comprising at least one IL-18Rα subunit (Sol(IL-18Rα) and/or Sol(IL-18Rα)ₓ as defined here above), and at least one IL-18Rβ subunit, as defined here after. The term “soluble receptor” has been defined above.

IL-18Rβ (also named AcPL, IL-18RacP, IL-1RacPL or IL-1R7 in the literature) is a member of the IL-1 receptor family and possesses an extracellular domain comprising three immunoglobulin-like domains (Ig domains). A cDNA encoding human IL-18Rβ is presented at SEQ ID NO: 3. This cDNA encodes a 599 amino acids long protein (SEQ ID NO: 4) which includes an extracellular domain of 356 amino acids (residues 1-356 from N- to C-terminus of SEQ ID NO: 4) that includes a signal peptide of 19 amino acids (residues 1-19 of SEQ ID NO: 4); a transmembrane region of 21 amino acids (residues 357-377) and a cytoplasmic domain of 222 amino acids (residues 378-599).

2.1 IL-18Rβ Subunit and Variants thereof (Named here after “Sol(IL-18Rβ)”):

In one aspect, the IL-18Rβ subunit of the soluble IL-18Rα receptor of the present invention is a polypeptides comprising all or part of the extracellular domain of IL-18Rβ, in particular all or part of the extracellular domain of human IL-18Rβ or a variant thereof.

In an aspect, the IL-18Rβ subunit of the soluble IL-18Rα receptor of the present invention (Sol(IL-18Rβ)) is a polypeptide comprising or consisting of amino acids residues 20-356 of SEQ ID NO: 4, or a variant of said polypeptide. Ordinarily, the variant polypeptides are at least 300 amino acids in length, often at least 325 amino acids in length, more often at least 337 amino acids in length. A variant is defined as a polypeptide having at least 80% amino acid sequence identity with the sequence of reference (here residues 20-356 of SEQ ID NO: 4), preferably at least 90% amino acid sequence identity, more preferably at least 95% amino acid sequence identity, more preferably at least 98% amino acid sequence identity and most preferably at least 99% amino acid sequence identity. More preferably, the variants are differing from the sequence of reference (here residues 20-356 of SEQ ID NO: 4) by five, more preferably by four, even more preferably by three, even more preferably by two and most preferably by one amino acid. In some particular aspects of the invention, the variants are differing from the sequence of reference (here residues 20-356 of SEQ ID NO: 4) by the lack of 20, 15, 10, 9, 8, 7, 6, 5, 4, 3, 2 or 1 amino acid(s) at the N-terminal and/or C-terminal end. One of skill in the art using the genetic code can readily determine polynucleotides that encode such polypeptides. “Percent (%) amino acid sequence identity” is defined as here above.

In another embodiment, Sol(IL-18Rβ) is a polypeptide comprising or consisting of amino acids residues 20-250, or 140-356, or 20-148 and 236-356 linked by a peptide bond, of SEQ ID NO: 4, or a variant of said polypeptide. Ordinarily, the variant polypeptides are at least 200 amino acids in length, often at least 217 amino acids in length, often at least 231 amino acids in length, more often at least 250 amino acids in length. A variant is defined as a polypeptide having at least 80% amino acid sequence identity with the sequence of reference (here residues 20-250, or 140-356, or 20-148 and 236-356 linked by a peptide bond, of SEQ ID NO: 4), preferably at least 90% amino acid sequence identity, more preferably at least 95% amino acid sequence identity, more preferably at least 98% amino acid sequence identity and most preferably at least 99% amino acid sequence identity. More preferably, the variants are differing from the sequence of reference (here residues 20-250, or 140-356, or 20-148 and 236-356 linked by a peptide bond, of SEQ ID NO: 4), by five, more preferably by four, even more preferably by three, even more preferably by two and most preferably by one amino acid. In some particular aspects of the invention, the variants are differing from the sequence of reference (here residues 20-250, or 140-356, or 20-148 and 236-356 linked by a peptide bond, of SEQ ID NO: 4), by the lack of 20, 15, 10, 9, 8, 7, 6, 5, 4, 3, 2 or 1 amino acid(s) at the N-terminal and/or C-terminal end. One of skill in the art using the genetic code can readily determine polynucleotides that encode such polypeptides. “Percent (%) amino acid sequence identity” is defined as here above.

In yet another embodiment, Sol(IL-18Rβ) is a polypeptide comprising or consisting of amino acids residues 20-148, or 140-250, or 236-356 of SEQ ID NO: 4, or a variant of said polypeptide. Ordinarily, the variant polypeptides are at least 100 amino acids in length, often at least 111 amino acids in length, often at least 121 amino acids in length, more often at least 129 amino acids in length. A variant is defined as a polypeptide having at least 80% amino acid sequence identity with the sequence of reference (here residues 20-148, or 140-250, or 236-356 of SEQ ID NO: 4), preferably at least 90% amino acid sequence identity, more preferably at least 95% amino acid sequence identity, more preferably at least 98% amino acid sequence identity and most preferably at least 99% amino acid sequence identity. More preferably, the variants are differing from the sequence of reference (here residues 20-148, or 140-250, or 236-356 of SEQ ID NO: 4) by five, more preferably by four, even more preferably by three, even more preferably by two and most preferably by one amino acid. In some particular aspects of the invention, the variants are differing from the sequence of reference (here residues 20-148, or 140-250, or 236-356 of SEQ ID NO: 4), by the lack of 20, 15, 10, 9, 8, 7, 6, 5, 4, 3, 2 or 1 amino acid(s) at the N-terminal and/or C-terminal end. One of skill in the art using the genetic code can readily determine polynucleotides that encode such polypeptides. “Percent (%) amino acid sequence identity” is defined as here above.

2.2 Soluble IL-18Rβ Comprising at Least Two IL-18Rβ Subunits or Variant thereof on the Same Protein Backbone (Named here after “Sol(IL-18Rβ)ₓ”):

As it will be described here after, the present invention, among other aspects, pertains to soluble IL-18Rα receptors comprising at least two IL-18Rβ subunits (at least two Sol(IL-18Rβ)). These soluble IL-18Rβ comprising at least two IL-18Rβ subunits (i.e at least two Sol(IL-18Rβ) subunits as defined here above) are on the same protein backbone as a fusion protein and are named here after “Sol(IL-18Rβ)ₓ”. In a particular embodiment, the fusion protein comprises two Sol(IL-18Rβ) subunits. In yet another particular embodiment, the at least two Sol(IL-18Rβ) subunit are the same (i.e the fusion protein is a homomultimer of Sol(IL-18Rβ)), and in a particular embodiment the fusion protein is a homodimer of Sol(IL-18Rβ).

The at least two IL-18Rβ subunit (Sol(IL-18Rβ)) are operably linked to one another. The term “operably linked” indicates that the subunits are associated through peptide linkage, either directly or via a “peptide linker”. In this manner, the fusion protein can be produced recombinantly, by direct expression in a host cell of a nucleic acid molecule encoding the same. The subunits are linked either directly or via a “peptide linker”. The peptide linker can be as short as 1 to 3 amino acid residues in length (preferably consisting of small amino acids such as glycine, serine, threonine or alanine) or longer, for example 13, 15 or 16 amino acid residues in length, introduced between the subunits. Preferably, the peptide linker is a peptide which is immunologically inert. Said linker may be a tripeptide of the sequence E-F-M (Glu-Phe-Met) (SEQ ID NO: 13), for example, a 13-amino acid linker sequence consisting of Glu-Phe-Gly-Ala-Gly-Leu-Val-Leu-Gly-Gly-Gln-Phe-Met (SEQ ID NO: 14), a 15-amino acid linker sequence consisting of (G₄S)₃ (SEQ ID NO: 15), a 16-amino acid linker sequence consisting of GGSGG SGGGG SGGGG S (SEQ ID NO: 16) or the hinge region of human IgG (e.g. IgG1, IgG2, IgG3 or IgG4).

2.3 Soluble IL-18Rα Comprising at Least One IL-18Rα Subunit (Sol(IL-18Rα) or Sol(IL-18Rα)ₓ) and at Least One IL-18Rβ Subunit (Sol(IL-18Rβ) or Sol(IL-18Rβ)ₓ):

As disclosed here above, the present invention, among other aspects, pertains to soluble IL-18Rα receptors comprising at least one IL-18Rα subunit ((Sol(IL-18Rα) or Sol(IL-18Rα)ₓ as defined here above), and one IL-18Rβ subunit (Sol(IL-18Rβ) or Sol(IL-18Rβ)ₓ as defined here above).

2.3.1 Soluble IL-18Rα Comprising at Least One IL-18Rα Subunit (Sol(IL-18Rα) or Sol(IL-18Rα)ₓ) and at Least One IL-18Rβ Subunit (Sol(IL-18Rβ) or Sol(IL-18Rβ)ₓ) on the Same Protein Backbone (Named here after “Sol(IL-18Rα)ₓ-(IL-18Rβ)ₓ”):

In one aspect of the present invention, the Sol(IL-18Rα) or Sol(IL-18Rα)ₓ, and, the Sol(IL-18Rβ) or Sol(IL-18Rβ)ₓ, are on the same protein backbone as a fusion protein (these soluble receptors will be named “Sol(IL-18Rα)ₓ-(IL-18Rβ)ₓ” here after). According to this embodiment, the Sol(IL-18Rα) or Sol(IL-18Rα)ₓ subunit is operably linked to the Sol(IL-18Rβ) or Sol(IL-18Rβ)ₓ subunit. The term “operably linked” indicates that the subunits are associated through peptide linkage, either directly or via a “peptide linker” (as defined here above). In this manner, the fusion protein can be produced recombinantly, by direct expression in a host cell of a nucleic acid molecule encoding the same. The Sol(IL-18Rα) or Sol(IL-18Rα)ₓ subunit can be located upstream (closer to the N-terminus of the protein) or downstream (closer to the C-terminus of the protein) to the Sol(IL-18Rβ) or Sol(IL-18Rβ)ₓ subunit. The subunits are linked either directly or via a “peptide linker”. In a particular embodiment, the fusion protein comprises one Sol(IL-18Rα) subunit and one Sol(IL-18Rβ) subunit as defined herein.

2.3.2 Soluble IL-18Rα Comprising at Least One IL-18Rα Subunit (Sol(IL-18Rα) or Sol(IL-18Rα)ₓ) and at Least One IL-18Rβ Subunit (Sol(IL-18Rβ) or Sol(IL-18Rβ)ₓ) on the Same Protein Backbone (Sol(IL-18Rα)ₓ-(IL-18Rβ)ₓ) as Fusion Protein:

In yet another particular aspect, the fusion protein comprising, the Sol(IL-18Rα) or Sol(IL-18Rα)ₓ, and, the Sol(IL-18Rβ) or Sol(IL-18Rβ)ₓ, subunits (Sol(IL-18Rα)ₓ-(IL-18Rβ)ₓ) is itself “operably linked” to an additional amino acid domain. The term “operably linked” indicates that the additional amino acid domain is associated through peptide linkage, either directly or via a “peptide linker” as defined here above. In this manner, this fusion protein can be produced recombinantly, by direct expression in a host cell of a nucleic acid molecule encoding the same. The additional amino acid domain may be located upstream (N-ter) or downstream (C-ter) to (Sol(IL-18Rα)ₓ-(IL-18Rβ)ₓ). In this embodiment, the additional amino acid domain comprises any functional region providing for instance an increased stability, targeting or bioavailability of the fusion protein; facilitating purification or production, or conferring on the molecule additional biological activity. Specific examples of such additional amino acid sequences include a GST sequence, a His tag sequence, the constant region of an immunoglobulin molecule or a heterodimeric protein hormone such as human chorionic gonadotropin (hCG) as described in U.S. Pat. No. 6,193,972. Also, if needed, the additional amino acid sequence included in the fusion proteins may be eliminated, either at the end of the production/purification process or in vivo, e.g., by means of an appropriate endo-/exopeptidase. For example, a spacer sequence included in the fusion protein may comprise a recognition site for an endopeptidase (such as a caspase) that can be used to separate by enzymatic cleavage the desired polypeptide variant from the additional amino acid domain, either in vivo or in vitro. In a particular aspect of this embodiment, (Sol(IL-18Rα)ₓ-(IL-18Rβ)ₓ) comprises one Sol(IL-18Rα) subunit and one Sol(IL-18Rβ) subunit as defined here above.

2.3.3 Multimers of Sol(IL-18Rα)ₓ-(IL-18Rβ)ₓ:

In a particular aspect, Sol(IL-18Rα)ₓ-(IL-18Rβ)ₓ soluble receptors are produced as multimers. Each subunit of the multimer comprising one Sol(IL-18Rα)ₓ-(IL-18Rβ)ₓ. These multimers may be homodimeric, heterodimeric, or multimeric soluble receptors, with multimeric receptors generally not comprising more than 9 subunits, preferably not comprising more than 6 subunits, even more preferably not more than 3 subunits and most preferably not comprising more than 2 subunits. Preferably, these multimers soluble receptors are homodimers of Sol(IL-18Rα)ₓ-(IL-18Rβ)ₓ as defined here above. In an embodiment, the subunits of the multimers are linked via covalent linkages. The subunits may be covalently linked by any suitable means, such as via a cross-linking reagent or a polypeptide linker. In another embodiment, the subunits are linked via non-covalent linkages.

In one embodiment, each Sol(IL-18Rα)ₓ-(IL-18Rβ)ₓ subunit is operably linked to an additional amino acid domain that provides for the multimerization of the subunits (in particular the additional domains comprise any functional region providing for dimerization of the subunits). The term “operably linked” is as defined here above. The additional amino acid domain may be located upstream (N-ter) or downstream (C-ter) from the sequence of the Sol(IL-18Rα)ₓ-(IL-18Rβ)ₓ subunit. In this manner, the fusion protein can be produced recombinantly, by direct expression in a host cell of a nucleic acid molecule encoding the same. In these embodiments, soluble IL-18Rα receptors of the invention are multimers of fusion proteins containing a Sol(IL-18Rα)ₓ-(IL-18Rβ)ₓ subunit, operably linked to a multimerizing component capable of interacting with the multimerizing component present in another fusion protein to form a higher order structure, such as a dimer. This type of fusion proteins may be prepared by operably linking the Sol(IL-18Rα)ₓ-(IL-18Rβ)ₓ subunit sequence to domains isolated from other proteins allowing the formation of dimers, trimers, etc. Examples for protein sequences allowing the multimerization of the IL-18Rα soluble receptors of the invention are domains isolated from proteins such as immunoglobulins, hCG (WO 97/30161), collagen X (WO 04/33486), C4BP (WO 04/20639), Erb proteins (WO 98/02540), or coiled coil peptides (WO 01/00814).

In a particular aspect, the multimers are dimers of Sol(IL-18Rα)ₓ-(IL-18Rβ)ₓ where the subunits are operably linked to an immunoglobulin. The term “operably linked” is as defined here above. In this embodiment, the subunits are operably linked to all or a portion of an immunoglobulin, particularly a human immunoglobulin, even more particularly the Fc portion of a human immunoglobulin. Typically an Fc portion of a human immunoglobulin contains two constant region domains (the CH2 and CH3 domains) and a hinge region but lacks the variable region (See e.g. U.S. Pat. Nos. 6,018,026 and 5,750,375). The immunoglobulin may be selected from any of the major classes of immunoglobulins, including IgA, IgD, IgE, IgG and IgM, and any subclass or isotype, e.g. IgG1, IgG2, IgG3 and IgG4; IgA-1 and IgA-2. In an embodiment, the Fc moiety is of human IgG4, which is stable in solution and has little or no complement activating activity. In another embodiment, the Fc moiety is of human IgG1. The Fc part may be mutated in order to prevent unwanted activities, such as complement binding, binding to Fc receptors, or the like. Usually the Sol(IL-18Rα)ₓ-(IL-18Rβ)ₓ subunits are operably linked to the same immunoglobulin (particularly to the Fc portion of a human immunoglobulin, for example of a human IgG4 or human IgG1). The amino acid sequence derived from the immunoglobulin may be linked to the C-terminus or to the N-terminus of Sol(IL-18Rα)ₓ-(IL-18Rβ)ₓ, preferably to the C-terminus. Such fusion proteins can be prepared by transfecting cells with DNA encoding Sol(IL-18Rα)ₓ-(IL-18Rβ)ₓ:Fc fusion protein and/or DNA encoding another Sol(IL-18Rα)ₓ-(IL-18Rβ)ₓ:Fc fusion protein and expressing the dimers in the same cells. In a particular embodiment, the subunits Sol(IL-18Rα)ₓ-(IL-18Rβ)ₓ are the same on each monomer (i.e the dimer is a homodimer of Sol(IL-18Rα)ₓ-(IL-18Rβ)ₓ). Even more particularly, the subunits of Sol(IL-18Rα)ₓ-(IL-18Rβ)ₓ are operably linked to the same immunoglobulin (particularly to the Fc portion of a human immunoglobulin, for example of a human IgG4 or human IgG1). Such fusion proteins can be prepared by transfecting cells with DNA encoding Sol(IL-18Rα)ₓ-(IL-18Rβ)ₓ:Fc fusion protein and expressing the dimers in the same cells. Subunits advantageously comprise a native or heterologous signal peptide when initially synthesized, to promote secretion from the cell, but the signal sequence is cleaved upon secretion. Methods for making immunoglobulin fusion proteins are well known in the art, such as the ones described in Hollenbaugh and Aruffo (“Construction of Immunoglobulin Fusion Proteins”, in Current Protocols in Immunology, Suppl. 4, pages 10.19.1-10.19.11, 1992) or WO 01/03737, for example.

Alternatively, the dimers of Sol(IL-18Rα)ₓ-(IL-18Rβ)ₓ of the present invention can be prepared by operably linking one of the receptor subunit to the constant region of an immunoglobulin heavy chain and operably linking the other receptor subunit to the constant region of an immunoglobulin light chain. The term “operably linked” indicates that Sol(IL-18Rα)ₓ-(IL-18Rβ)ₓ, and the immunoglobulin are associated through peptide linkage, either directly or via a “peptide linker” (as defined here above). For example, a Sol(IL-18Rα)ₓ-(IL-18Rβ)ₓ subunit can be operably linked to the CH₁-hinge-CH2-CH3 region of human IgG1 and another or the same Sol(IL-18Rα)ₓ-(IL-18Rβ)ₓ subunit can be operably linked to the C kappa region of the Ig kappa light chain. The amino acid sequence derived from the immunoglobulin may be linked to the C-terminus or to the N-terminus of the Sol(IL-18Rα)ₓ-(IL-18Rβ)ₓ subunits, preferably to the C-terminus. Cells transfected with DNA encoding the immunoglobulin light chain fusion protein and the immunoglobulin heavy chain fusion protein express heavy chain/light chain heterodimers containing each a Sol(IL-18Rα)ₓ-(IL-18Rβ)ₓ subunit. Both subunits advantageously comprise a native or heterologous signal peptide when initially synthesized, to promote secretion from the cell, but the signal sequence is cleaved upon secretion. In a particular embodiment, the subunits Sol(IL-18Rα)ₓ-(IL-18Rβ)ₓ are the same on each monomer (i.e the dimer is a homodimer of Sol(IL-18Rα)ₓ-(IL-18Rβ)ₓ).

In another particular aspect of the present invention, the subunits of the multimers Sol(IL-18Rα)ₓ-(IL-18Rβ)ₓ (as defined here above) are linked via non-covalent linkages. Non-covalent bonding of the subunits may be achieved by any suitable means that does not interfere with its biological activity (i.e. its ability to reduce the symptoms of MS). In a particular aspect, these multimers are dimers of Sol(IL-18Rα)ₓ-(IL-18Rβ)ₓ where one subunit of Sol(IL-18Rα)ₓ-(IL-18Rβ)ₓ is operably linked to a first compound and another or the same subunit Sol(IL-18Rα)ₓ-(IL-18Rβ)ₓ is operably linked to a second compound that will non-covalently bond to the first compound. The term “operably linked” is as defined here above. Examples of such compounds are biotin and avidin. The dimers of Sol(IL-18Rα)ₓ-(IL-18Rβ)ₓ can be prepared by operably linking one of the receptor subunit to biotin and operably linking the other subunit to avidin. The receptor is thus formed through the non-covalent interactions of biotin with avidin. Other examples include subunits of heterodimeric proteinaceous hormone. In these embodiments, a DNA construct encoding one subunit of Sol(IL-18Rα)ₓ-(IL-18Rβ)ₓ is fused to a DNA construct encoding a subunit of a heterodimeric proteinaceous hormone, such as hCG, and a DNA construct encoding the other Sol(IL-18Rα)ₓ-(IL-18Rβ)ₓ subunit is fused to DNA encoding the other subunit of the heterodimeric proteinaceous hormone, such as hCG (as disclosed in U.S. Pat. No. 6,193,972). These DNA constructs are coexpressed in the same cells leading to the expression of an Sol(IL-18Rα)ₓ-(IL-18Rβ)ₓ heterodimeric receptor fusion protein, as each coexpressed sequence contains a corresponding hormone subunit so as to form a heterodimer upon expression. The amino acid sequence derived from the heterodimeric proteinaceous hormone may be linked to the C-terminus or to the N-terminus of the Sol(IL-18Rα)ₓ-(IL-18Rβ)ₓ subunits, preferably to the C-terminus. Both subunits advantageously comprise a native or heterologous signal peptide when initially synthesized, to promote secretion from the cell, but the signal sequence is cleaved upon secretion. In a particular embodiment, the subunits Sol(IL-18Rα)ₓ-(IL-18Rβ)ₓ are the same on each monomer (i.e the dimer is a homodimer of Sol(IL-18Rα)ₓ-(IL-18Rβ)ₓ).

Other examples for protein sequences allowing the dimerization of the Sol(IL-18Rα)ₓ-(IL-18Rβ)ₓ subunits are domains isolated from proteins such as collagen X (WO 04/33486), C4BP (WO 04/20639), Erb proteins (WO 98/02540), or coiled coil peptides (WO 01/00814).

Also, if needed, fusion proteins described herein may comprise any functional region facilitating purification or production. Specific examples of such additional amino acid sequences include a GST sequence or a His tag sequence.

2.3.4 Soluble IL-18Rα Comprising at Least One IL-18Rα Subunit (Sol(IL-18Rα) or Sol(IL-18Rα)ₓ) and at Least One IL-18Rβ Subunit (Sol(IL-18Rβ) or Sol(IL-18Rβ)ₓ) as heteromultimers:

In a particular aspect, soluble receptors of the present invention comprising at least one IL-18Rα subunit (Sol(IL-18Rα) or Sol(IL-18Rα)ₓ) and at least one IL-18Rβ subunit (Sol(IL-18Rβ) or Sol(IL-18Rβ)ₓ) are heteromultimers. Each subunit of the heteromultimer comprising:

at least one IL-18Rα subunit (Sol(IL-18Rα) or Sol(IL-18Rα)ₓ) or;

at least one IL-18Rβ subunit (Sol(IL-18Rβ) or Sol(IL-18Rβ)ₓ).

These heteromultimers generally do not comprise more than 9 subunits, preferably not more than 6 subunits, even more preferably not more than 3 subunits and most preferably not more than 2 subunits. Preferably, these heteromultimers soluble receptors are heterodimers comprising one subunit consisting of Sol(IL-18Rα) or Sol(IL-18Rα)ₓ (as defined above) and one subunit consisting of Sol(IL-18Rβ) or Sol(IL-18Rβ)ₓ (as defined above). In an embodiment, the subunits of the heteromultimers are linked via covalent linkages. The subunits may be covalently linked by any suitable means, such as via a cross-linking reagent. In another embodiment, the subunits are linked via non-covalent linkages.

In one embodiment, each subunit of the heteromultimer is operably linked to an additional amino acid domain that provides for the multimerization of the subunits (in particular the additional domains may comprise any functional region providing for dimerization of the subunits). The term “operably linked” is as defined here above. The additional amino acid domain may be located upstream (N-ter) or downstream (C-ter) (preferably downstream (C-ter)) from the sequence of the Sol(IL-18Rα) or Sol(IL-18Rα)ₓ subunit(s) and upstream (N-ter) or downstream (C-ter) (preferably downstream (C-ter)) from the sequence of the Sol(IL-18Rβ) or Sol(IL-18Rβ)ₓ subunit(s). In this manner, the fusion protein can be produced recombinantly, by direct expression in a host cell of a nucleic acid molecule encoding the same. In these embodiments, soluble IL-18Rα receptors of the invention are heteromultimers of fusion proteins containing one subunit consisting of Sol(IL-18Rα) or Sol(IL-18Rα)ₓ or of Sol(IL-18Rβ) or Sol(IL-18Rβ)ₓ, operably linked to a multimerizing component capable of interacting with the multimerizing component present in another fusion protein to form a higher order structure, such as a dimer. This type of fusion proteins may be prepared by operably linking the Sol(IL-18Rα) or Sol(IL-18Rα)ₓ subunit sequence and the Sol(IL-18Rβ) or Sol(IL-18Rβ)ₓ subunit sequence to domains isolated from other proteins allowing the formation of dimers, trimers, etc. Examples for protein sequences allowing the multimerization of the IL-18Rα soluble receptors of the invention are domains isolated from proteins such as immunoglobulins, hCG (WO 97/30161), collagen X (WO 04/33486), C4BP (WO 04/20639), Erb proteins (WO 98/02540), or coiled coil peptides (WO 01/00814).

In a particular aspect, the heteromultimers are heterodimers comprising one subunit consisting of Sol(IL-18Rα) and one subunit consisting of Sol(IL-18Rβ), or one subunit consisting of Sol(IL-18Rα)ₓ and one subunit consisting of Sol(IL-18Rβ), or one subunit consisting of Sol(IL-18Rα) and one subunit consisting of Sol(IL-18Rβ)ₓ, or one subunit consisting of Sol(IL-18Rα)ₓ and one subunit consisting of Sol(IL-18Rβ)ₓ. In yet another particular aspect, the two subunits of the heterodimer are operably linked to an immunoglobulin. The term “operably linked” is as defined here above. In these embodiment, the subunits are operably linked to all or a portion of an immunoglobulin, particularly a human immunoglobulin, even more particularly the Fc portion of a human immunoglobulin. Typically an Fc portion of a human immunoglobulin contains two constant region domains (the CH2 and CH3 domains) and a hinge region but lacks the variable region (See e.g. U.S. Pat. Nos. 6,018,026 and 5,750,375). The immunoglobulin may be selected from any of the major classes of immunoglobulins, including IgA, IgD, IgE, IgG and IgM, and any subclass or isotype, e.g. IgG1, IgG2, IgG3 and IgG4; IgA-1 and IgA-2. In an embodiment, the Fc moiety is of human IgG4, which is stable in solution and has little or no complement activating activity. In another embodiment, the Fc moiety is of human IgG1. The Fc part may be mutated in order to prevent unwanted activities, such as complement binding, binding to Fc receptors, or the like. Usually the two subunits are operably linked to the same immunoglobulin (particularly to the Fc portion of a human immunoglobulin, for example of a human IgG4 or human IgG1). The amino acid sequence derived from the immunoglobulin may be linked to the C-terminus or to the N-terminus of the subunit, preferably to the C-terminus. Such fusion proteins can be prepared by transfecting cells with DNA encoding the first subunit:Fc fusion protein and DNA encoding the other subunit:Fc fusion protein and expressing the dimers in the same cells. Subunits advantageously comprise a native or heterologous signal peptide when initially synthesized, to promote secretion from the cell, but the signal sequence is cleaved upon secretion. Methods for making immunoglobulin fusion proteins are well known in the art, such as the ones described in Hollenbaugh and Aruffo (“Construction of Immunoglobulin Fusion Proteins”, in Current Protocols in Immunology, Suppl. 4, pages 10.19.1-10.19.11, 1992) or WO 01/03737, for example.

Alternatively, the heterodimers comprising one subunit consisting of Sol(IL-18Rα) and one subunit consisting of Sol(IL-18Rβ), or one subunit consisting of Sol(IL-18Rα)ₓ and one subunit consisting of Sol(IL-18Rβ), or one subunit consisting of Sol(IL-18Rα) and one subunit consisting of Sol(IL-18Rβ)ₓ, or one subunit consisting of Sol(IL-18Rα)ₓ and one subunit consisting of Sol(IL-18Rβ)ₓ, of the present invention can be prepared by operably linking one of the receptor subunit to the constant region of an immunoglobulin heavy chain and operably linking the other receptor subunit to the constant region of an immunoglobulin light chain. The term “operably linked” is as defined here above. For example, the Sol(IL-18Rα) or Sol(IL-18Rα)ₓ subunit can be operably linked to the CH₁-hinge-CH2-CH3 region of human IgG1 and the Sol(IL-18Rβ) subunit can be operably linked to the C kappa region of the Ig kappa light chain (or vice versa); or the Sol(IL-18Rα) or Sol(IL-18Rα)ₓ subunit can be operably linked to the CH₁-hinge-CH2-CH3 region of human IgG1 and the Sol(IL-18Rβ)ₓ subunit can be operably linked to the C kappa region of the Ig kappa light chain (or vice versa). The amino acid sequence derived from the immunoglobulin may be linked to the C-terminus or to the N-terminus of the subunits, preferably to the C-terminus. Cells transfected with DNA encoding the immunoglobulin light chain fusion protein and the immunoglobulin heavy chain fusion protein express heavy chain/light chain heterodimers containing each a subunit. Both subunits advantageously comprise a native or heterologous signal peptide when initially synthesized, to promote secretion from the cell, but the signal sequence is cleaved upon secretion.

In another particular aspect of the present invention, the subunits of the heteromultimers are linked via non-covalent linkages. Non-covalent bonding of the subunits may be achieved by any suitable means that does not interfere with its biological activity (i.e. its ability to reduce the symptoms of MS). In a particular aspect, these heteromultimers are heterodimers comprising one subunit consisting of Sol(IL-18Rα) and one subunit consisting of Sol(IL-18Rβ), or one subunit consisting of Sol(IL-18Rα)ₓ and one subunit consisting of Sol(IL-18Rβ), or one subunit consisting of Sol(IL-18Rα) and one subunit consisting of Sol(IL-18Rβ)ₓ, or one subunit consisting of Sol(IL-18Rα)ₓ and one subunit consisting of Sol(IL-18Rβ)ₓ, where one subunit is operably linked to a first compound the other is operably linked to a second compound that will non-covalently bond to the first compound. The term “operably linked” is as defined here above. Examples of such compounds are biotin and avidin. These heterodimers can be prepared by operably linking one of the receptor subunit to biotin and operably linking the other subunit to avidin. The receptor is thus formed through the non-covalent interactions of biotin with avidin. Other examples include subunits of heterodimeric proteinaceous hormone. In these embodiments, a DNA construct encoding one subunit (Sol(IL-18Rα) or Sol(IL-18Rα)ₓ) is fused to a DNA construct encoding a subunit of a heterodimeric proteinaceous hormone, such as hCG, and a DNA construct encoding the other subunit (Sol(IL-18Rβ) or Sol(IL-18Rβ)ₓ) is fused to DNA encoding the other subunit of the heterodimeric proteinaceous hormone, such as hCG (as disclosed in U.S. Pat. No. 6,193,972). These DNA constructs are coexpressed in the same cells leading to the expression of an heterodimeric receptor fusion protein, as each coexpressed sequence contains a corresponding hormone subunit so as to form a heterodimer upon expression. The amino acid sequence derived from the heterodimeric proteinaceous hormone may be linked to the C-terminus or to the N-terminus of the subunits, preferably to the C-terminus. Both subunits advantageously comprise a native or heterologous signal peptide when initially synthesized, to promote secretion from the cell, but the signal sequence is cleaved upon secretion.

Other examples for protein sequences allowing the dimerization of the Sol(IL-18Rα)ₓ-(IL-18Rβ)ₓ subunits are domains isolated from proteins such as collagen X (WO 04/33486), C4BP (WO 04/20639), Erb proteins (WO 98/02540), or coiled coil peptides (WO 01/00814).

In an embodiment, the heteromultimers comprising at least one Sol(IL-18Rα) or Sol(IL-18Rα)ₓ subunit and one Sol(IL-18Rβ) or Sol(IL-18Rβ)ₓ subunit of the present invention are recombinant antibodies. The technology of recombinant antibody is described for example in the U.S. Pat. No. 6,018,026. In that case, the multimer of one Sol(IL-18Rα) or Sol(IL-18Rα)ₓ and Sol(IL-18Rβ) or Sol(IL-18Rβ)ₓ is a multimer polypeptide fusion, comprising: a first Sol(IL-18Rα) or Sol(IL-18Rα)ₓ polypeptide chain and a second Sol(IL-18Rβ) or Sol(IL-18Rβ)ₓ polypeptide chains, wherein one of the polypeptide chain is operably linked to an immunoglobulin heavy chain constant region and the other polypeptide chain is operably linked to an immunoglobulin light chain constant region. In an embodiment, the first Sol(IL-18Rα) or Sol(IL-18Rα)ₓ polypeptide chain is operably linked to an immunoglobulin heavy chain constant region and the second Sol(IL-18Rβ) or Sol(IL-18Rβ)ₓ polypeptide chains is operably linked to an immunoglobulin light chain constant region. In another embodiment, the first Sol(IL-18Rα) or Sol(IL-18Rα)ₓ polypeptide chain is operably linked to an immunoglobulin light chain constant region and the second Sol(IL-18Rβ) or Sol(IL-18Rβ)ₓ polypeptide chains is operably linked to an immunoglobulin heavy chain constant region. The term “operably linked” indicates that Sol(IL-18Rα) or Sol(IL-18Rα)ₓ and Sol(IL-18Rβ) or Sol(IL-18Rβ)ₓ, and the immunoglobulin heavy or light chain constant region are associated through peptide linkage, either directly or via a “peptide linker” (as defined here above). In an embodiment, the immunoglobulin heavy chain constant region domain and the immunoglobulin light chain constant region domain are human immunoglobulin constant regions. In an embodiment, the immunoglobulin heavy chain constant region domain is selected from the group consisting of the constant region of an α, γ, μ, δ or ε human immunoglobulin heavy chain. Preferably, said constant region is the constant region of a γ1, γ2, γ3 or γ4 human immunoglobulin heavy chain. In a preferred embodiment, the immunoglobulin light chain constant region domain is selected from the group consisting of the constant region of a κ or λ human immunoglobulin light chain. The amino acid sequence from the immunoglobulin may be linked to the C-terminus or to the N-terminus of the Sol(IL-18Rα) or Sol(IL-18Rα)ₓ and Sol(IL-18Rβ) or Sol(IL-18Rβ)ₓ subunits, preferably to the C-terminus. Cells transfected with DNA encoding the immunoglobulin light chain fusion protein and the immunoglobulin heavy chain fusion protein express a fusion protein having the structure of an antibody. The resulting protein obtained consists of:

two identical heavy chains constant region operably linked to a Sol(IL-18Rα) or Sol(IL-18Rα)ₓ subunit and two identical light chains constant region operably linked to a Sol(IL-18Rβ) or Sol(IL-18Rβ)ₓ subunit; or

two identical heavy chains constant region operably linked to a Sol(IL-18Rβ) or Sol(IL-18Rβ)ₓ subunit and two identical light chains constant region operably linked to a Sol(IL-18Rα) or Sol(IL-18Rα)ₓ subunit.

As for an antibody, heavy and light chains are disulfide linked (interchain disulfide bond) and heavy chains are disulfide linked (interchain disulfide bond).
The resulting molecule is therefore a homodimer composed of two heterodimers each of these heterodimers being composed of:

an immunoglobulin heavy chain constant region operably linked to a Sol(IL-18Rα) or Sol(IL-18Rα)ₓ polypeptide chain and;

an immunoglobulin light chain constant region operably linked to a Sol(IL-18Rβ) or Sol(IL-18Rβ)ₓ polypeptide chain. Or a homodimer composed of two heterodimers each of these heterodimers being composed of:

an immunoglobulin heavy chain constant region operably linked to a Sol(IL-18Rβ) or Sol(IL-18Rβ)ₓ polypeptide chain and;

an immunoglobulin light chain constant region operably linked to a Sol(IL-18Rα) or Sol(IL-18Rα)ₓ polypeptide chain.
Both subunits advantageously comprise a native or heterologous signal peptide when initially synthesized, to promote secretion from the cell, but the signal sequence is cleaved upon secretion. In an embodiment, the heavy constant chain is human γ4, which is stable in solution and has little or no complement activating activity. In another embodiment, the heavy constant chain is human γ1. The heavy constant chain may be mutated in order to prevent unwanted activities, such as complement binding, binding to Fc receptors, or the like.

1. In an embodiment the recombinant antibody of the present invention comprises or consists of:

two identical heavy chains constant regions, said heavy chains constant regions being the constant region of γ1, γ2, γ3 or γ4 human immunoglobulin heavy chain, operably linked to the extracellular domain of the human IL-18Rα and;

two identical light chains constant region, said light chain constant region being the constant region of κ or λ human immunoglobulin light chain, operably linked to the extra cellular domain of the human IL-18Rβ. In an embodiment, heavy and light chains are disulfide linked (interchain disulfide bond) and heavy chains are disulfide linked (interchain disulfide bond) as for a natural antibody.

2. In another particular embodiment, the recombinant antibody of the present invention comprises or consists of:

two identical heavy chains constant region, said heavy chains constant region being the constant region of γ1, γ2, γ3 or γ4 human immunoglobulin heavy chain, operably linked to the extracellular domain of the human IL-18Rβ and;

two identical light chains constant region, said light chain constant region being the constant region of κ or λ human immunoglobulin light chain, operably linked to the extra cellular domain of the human IL-18Rα. In an embodiment, heavy and light chains are disulfide linked (interchain disulfide bond) and heavy chains are disulfide linked (interchain disulfide bond) as for a natural antibody.

3. In another embodiment, the present invention resides in a recombinant antibody as defined at point 1 or 2 above wherein the constant regions of the heavy chain are the constant regions of γ1 human immunoglobulin heavy chain.

4. In another embodiment, the present invention resides in a recombinant antibody as defined at point 1, 2 or 3 above wherein the constant regions of the light chain are the constant regions of κ human immunoglobulin light chain.

5. In another embodiment, the present invention resides in a recombinant antibody as defined at point 1, 2, 3 or 4 above wherein the extra cellular domain of the human IL-18Rα consists of amino acids residues 19-329 of SEQ ID NO: 2 or a variant of said polypeptide as defined here above.

6. In another embodiment, the present invention resides in a recombinant antibody as defined at point 1, 2, 3, 4 or 5 above wherein the extra cellular domain of the human IL-18Rβ consists of amino acids residues 20-356 of SEQ ID NO: 4 or a variant of said polypeptide as defined here above.

7. In another embodiment, the present invention resides in a recombinant antibody as defined at point 1, 2, 3, 4, 5 or 6 above wherein the heavy chain constant regions are directly associated through peptide linkage to the extracellular domain of the human IL-18Rα or of the human IL-18Rβ.

8. In another embodiment, the present invention resides in a recombinant antibody as defined at point 1, 2, 3, 4, 5, 6 or 7 above wherein the light chain constant regions are directly associated through peptide linkage to the extracellular domain of the human IL-18Rα or of the human IL-18Rβ.

9. In another embodiment, the present invention resides in a recombinant antibody as defined at point 1, 2, 3, 4, 5 or 6 above wherein the heavy chain constant regions are associated through peptide linkage via a peptide linker to the extracellular domain of the human IL-18Rα or of the human IL-18Rβ. The peptide linker can be as short as 1 to 3 amino acid residues in length (preferably consisting of small amino acids such as glycine, serine, threonine or alanine) or longer, for example 13, 15 or 16 amino acid residues in length, introduced between the subunits. Preferably, the peptide linker is a peptide which is immunologically inert. Said linker may be a tripeptide of the sequence E-F-M (Glu-Phe-Met) (SEQ ID NO: 13), for example, a 13-amino acid linker sequence consisting of Glu-Phe-Gly-Ala-Gly-Leu-Val-Leu-Gly-Gly-Gln-Phe-Met (SEQ ID NO: 14), a 15-amino acid linker sequence consisting of (G₄S)₃ (SEQ ID NO: 15), a 16-amino acid linker sequence consisting of GGSGG SGGGG SGGGG S (SEQ ID NO: 16) or the hinge region of human IgG (e.g. IgG1, IgG2, IgG3 or IgG4). In an embodiment, said peptide linker is a 15-amino acid linker sequence consisting of (G₄S)₃ (SEQ ID NO: 15),

10. In another embodiment, the present invention resides in a recombinant antibody as defined at point 1, 2, 3, 4, 5, 6 or 9 above wherein the light chain constant regions are associated through peptide linkage via a peptide linker to the extracellular domain of the human IL-18Rα or of the human IL-18Rβ. The peptide linker can be as short as 1 to 3 amino acid residues in length (preferably consisting of small amino acids such as glycine, serine, threonine or alanine) or longer, for example 13, 15 or 16 amino acid residues in length, introduced between the subunits. Preferably, the peptide linker is a peptide which is immunologically inert. Said linker may be a tripeptide of the sequence E-F-M (Glu-Phe-Met) (SEQ ID NO: 13), for example, a 13-amino acid linker sequence consisting of Glu-Phe-Gly-Ala-Gly-Leu-Val-Leu-Gly-Gly-Gln-Phe-Met (SEQ ID NO: 14), a 15-amino acid linker sequence consisting of (G₄S)₃ (SEQ ID NO: 15), a 16-amino acid linker sequence consisting of GGSGG SGGGG SGGGG S (SEQ ID NO: 16) or the hinge region of human IgG (e.g. IgG1, IgG2, IgG3 or IgG4). In an embodiment, said peptide linker is a 15-amino acid linker sequence consisting of (G₄S)₃ (SEQ ID NO: 15).

11. In another embodiment, the present invention resides in a recombinant antibody as defined at point 1, 2, 3, 4, 5, 6, 7, 8, 9 or 10 above wherein the heavy constant chain is human γ4, which is stable in solution and has little or no complement activating activity.

12. In another embodiment, the present invention resides in a recombinant antibody as defined at point 1, 2, 3, 4, 5, 6, 7, 8, 9 or 10 above wherein the heavy constant chain is human γ1 and is mutated in order to prevent unwanted activities, such as complement binding, binding to Fc receptors, or the like.

13. In another embodiment, the present invention resides in a recombinant antibody as defined at point 1, 2, 3, 4, 5, 6, 7, 8, 9, 10, 11 or 12 above wherein the heavy chain constant regions are operably linked to the C-terminus or to the N-terminus of the extracellular domain of the human IL-18Rα or of the human IL-18Rβ, preferably to the C-terminus.

14. In another embodiment, the present invention resides in a recombinant antibody as defined at point 1, 2, 3, 4, 5, 6, 7, 8, 9, 10, 11, 12 or 13 above wherein the light chain constant regions are operably linked to the C-terminus or to the N-terminus of the extracellular domain of the human IL-18Rα or of the human IL-18Rβ, preferably to the C-terminus.

15. In another embodiment, the present invention resides in a recombinant antibody as defined at point 1, 2, 3, 4, 5, 6, 7, 8, 9, 10, 11, 12, 13 or 14 above wherein the extracellular domain of the human IL-18Rα or of the human IL-18Rβ is operably linked to the C-terminus or to the N-terminus of the heavy chain constant regions, preferably to the N-terminus.

16. In another embodiment, the present invention resides in a recombinant antibody as defined at point 1, 2, 3, 4, 5, 6, 7, 8, 9, 10, 11, 12, 13, 14 or 15 above wherein the extracellular domain of the human IL-18Rα or of the human IL-18Rβ is operably linked to the C-terminus or to the N-terminus of the light chain constant regions, preferably to the N-terminus.

Also, if needed, fusion proteins described herein may comprise any functional region facilitating purification or production. Specific examples of such additional amino acid sequences include a GST sequence or a His tag sequence.

3) Soluble IL-18Rα Comprising at Least One IL-18Rα Subunit (Sol(IL-18Rα) and/or Sol(IL-18Rα)ₓ) and One IL-1RAcP Subunit (Sol(IL-1RAcP) and/or Sol(IL-1RAcP)ₓ):

In a particular aspect of the present invention, the soluble IL-18Rα receptors used to treat, prevent or ameliorate the symptoms of an autoimmune or demyelinating disease, in particular MS, are soluble receptors comprising at least one IL-18Rα subunit (Sol(IL-18Rα) and/or Sol(IL-18Rα)ₓ as defined here above), and at least one IL-1RAcP subunit, as defined here after. The term “soluble receptor” has been defined above.

IL-1RAcP (also named IL1RAP, FLJ37788 or IL1R3 in the literature) is a member of the IL-1 receptor family and possesses an extracellular domain comprising three immunoglobulin-like domains (Ig domains). A cDNA encoding human IL-1RAcP is presented at SEQ ID NO: 5. This cDNA encodes a 570 amino acids long protein (SEQ ID NO: 6) which includes an extracellular domain of 367 amino acids (residues 1-367 from N- to C-terminus of SEQ ID NO: 6) that includes a signal peptide of 20 amino acids (residues 1-20 of SEQ ID NO: 6); a transmembrane region of 21 amino acids (residues 368-388) and a cytoplasmic domain of 182 amino acids (residues 389-570).

3.1 IL-1RAcP Subunit and Variants thereof (Named here after “Sol(IL-1RAcP)”):

In one aspect, the IL-1RAcP subunit of the soluble IL-18Rα receptor of the present invention is a polypeptide comprising all or part of the extracellular domain of IL-1RAcP, in particular all or part of the extracellular domain of human IL-1RAcP or a variant thereof.

In an aspect, the IL-1RAcP subunit of the soluble IL-18Rα receptor of the present invention (Sol(IL-1RAcP)) is a polypeptide comprising or consisting of amino acids residues 21-367 of SEQ ID NO: 6, or a variant of said polypeptide. Ordinarily, the variant polypeptides are at least 300 amino acids in length, often at least 325 amino acids in length, more often at least 347 amino acids in length. A variant is defined as a polypeptide having at least 80% amino acid sequence identity with the sequence of reference (here residues 21-367 of SEQ ID NO: 6), preferably at least 90% amino acid sequence identity, more preferably at least 95% amino acid sequence identity, more preferably at least 98% amino acid sequence identity and most preferably at least 99% amino acid sequence identity. More preferably, the variants are differing from the sequence of reference (here residues 21-367 of SEQ ID NO: 6) by five, more preferably by four, even more preferably by three, even more preferably by two and most preferably by one amino acid. In some particular aspects of the invention, the variants are differing from the sequence of reference (here residues 21-367 of SEQ ID NO: 6) by the lack of 20, 15, 10, 9, 8, 7, 6, 5, 4, 3, 2 or 1 amino acid(s) at the N-terminal and/or C-terminal end. One of skill in the art using the genetic code can readily determine polynucleotides that encode such polypeptides. “Percent (%) amino acid sequence identity” is defined as here above.

In another embodiment, Sol(IL-1RAcP) is a polypeptide comprising or consisting of amino acids residues 21-241, or 129-367, or 21-140 and 231-367 linked by a peptide bond, of SEQ ID NO: 6, or a variant of said polypeptide. Ordinarily, the variant polypeptides are at least 200 amino acids in length, often at least 221 amino acids in length, often at least 239 amino acids in length, more often at least 257 amino acids in length. A variant is defined as a polypeptide having at least 80% amino acid sequence identity with the sequence of reference (here residues 21-241, or 129-367, or 21-140 and 231-367 linked by a peptide bond, of SEQ ID NO: 6), preferably at least 90% amino acid sequence identity, more preferably at least 95% amino acid sequence identity, more preferably at least 98% amino acid sequence identity and most preferably at least 99% amino acid sequence identity. More preferably, the variants are differing from the sequence of reference (here residues 21-241, or 129-367, or 21-140 and 231-367 linked by a peptide bond, of SEQ ID NO: 6), by five, more preferably by four, even more preferably by three, even more preferably by two and most preferably by one amino acid. In some particular aspects of the invention, the variants are differing from the sequence of reference (here residues 21-241, or 129-367, or 21-140 and 231-367 linked by a peptide bond, of SEQ ID NO: 6), by the lack of 20, 15, 10, 9, 8, 7, 6, 5, 4, 3, 2 or 1 amino acid(s) at the N-terminal and/or C-terminal end. One of skill in the art using the genetic code can readily determine polynucleotides that encode such polypeptides. “Percent (%) amino acid sequence identity” is defined as here above.

In yet another embodiment, Sol(IL-1RAcP) is a polypeptide comprising or consisting of amino acids residues 21-140, or 129-241, or 231-367 of SEQ ID NO: 6, or a variant of said polypeptide. Ordinarily, the variant polypeptides are at least 100 amino acids in length, often at least 113 amino acids in length, often at least 120 amino acids in length, more often at least 137 amino acids in length. A variant is defined as a polypeptide having at least 80% amino acid sequence identity with the sequence of reference (here residues 21-140, or 129-241, or 231-367 of SEQ ID NO: 6), preferably at least 90% amino acid sequence identity, more preferably at least 95% amino acid sequence identity, more preferably at least 98% amino acid sequence identity and most preferably at least 99% amino acid sequence identity. More preferably, the variants are differing from the sequence of reference (here residues 21-140, or 129-241, or 231-367 of SEQ ID NO: 6) by five, more preferably by four, even more preferably by three, even more preferably by two and most preferably by one amino acid. In some particular aspects of the invention, the variants are differing from the sequence of reference (here residues 21-140, or 129-241, or 231-367 of SEQ ID NO: 6), by the lack of 20, 15, 10, 9, 8, 7, 6, 5, 4, 3, 2 or 1 amino acid(s) at the N-terminal and/or C-terminal end. One of skill in the art using the genetic code can readily determine polynucleotides that encode such polypeptides. “Percent (%) amino acid sequence identity” is defined as here above.

3.2 Soluble IL-1RAcP Comprising at Least Two IL-1RAcP Subunits or Variant thereof on the Same Protein Backbone (Named here after “Sol(IL-1RAcP)ₓ”):

As it will be described here after, the present invention, among other aspects, pertains to soluble IL-18Rα receptors comprising at least two IL-1RAcP subunits (at least two Sol(IL-1RAcP)). These soluble IL-1RAcP comprising at least two IL-1RAcP subunits (i.e at least two Sol(IL-1RAcP) subunits as defined here above) are on the same protein backbone as a fusion protein and are named here after “Sol(IL-1RAcP)ₓ”. In a particular embodiment, the fusion protein comprises two Sol(IL-1RAcP) subunits. In yet another particular embodiment, the at least two Sol(IL-1RAcP) subunits are the same (i.e the fusion protein is a homomultimer of Sol(IL-1RAcP)), and in a particular embodiment the fusion protein is a homodimer of Sol(IL-1RAcP).

The at least two IL-1RAcP subunits are operably linked to one another. The term “operably linked” indicates that the subunits are associated through peptide linkage, either directly or via a “peptide linker”. In this manner, the fusion protein can be produced recombinantly, by direct expression in a host cell of a nucleic acid molecule encoding the same. The subunits are linked either directly or via a “peptide linker”. The peptide linker can be as short as 1 to 3 amino acid residues in length (preferably consisting of small amino acids such as glycine, serine, threonine or alanine) or longer, for example 13, 15 or 16 amino acid residues in length, introduced between the subunits. Preferably, the peptide linker is a peptide which is immunologically inert. Said linker may be a tripeptide of the sequence E-F-M (Glu-Phe-Met) (SEQ ID NO: 13), for example, a 13-amino acid linker sequence consisting of Glu-Phe-Gly-Ala-Gly-Leu-Val-Leu-Gly-Gly-Gln-Phe-Met (SEQ ID NO: 14), a 15-amino acid linker sequence consisting of (G₄S)₃ (SEQ ID NO: 15), a 16-amino acid linker sequence consisting of GGSGG SGGGG SGGGG S (SEQ ID NO: 16) or the hinge region of human IgG (e.g. IgG1, IgG2, IgG3 or IgG4).

3.3 Soluble IL-18Rα Comprising at Least One IL-18Rα Subunit (Sol(IL-18Rα) or Sol(IL-18Rα)ₓ) and at Least One IL-1RAcP Subunit (Sol(IL-1RAcP) or Sol(IL-1RAcP)ₓ):

As disclosed here above, the present invention, among other aspects, pertains to soluble IL-18Rα receptors comprising at least one IL-18Rα subunit ((Sol(IL-18Rα) or Sol(IL-18Rα)ₓ as defined here above), and one IL-1RAcP subunit (Sol(IL-1RAcP) or Sol(IL-1RAcP)ₓ as defined here above).

3.3.1 Soluble IL-18Rα Comprising at Least One IL-18Rα Subunit (Sol(IL-18Rα) or Sol(IL-18Rα)ₓ) and at Least One IL-1RAcP Subunit (Sol(IL-1RAcP) or Sol(IL-1RAcP)ₓ) on the Same Protein Backbone (Named here after “Sol(IL-18Rα)ₓ-(IL-1RAcP)ₓ”):

In one aspect of the present invention, the Sol(IL-18Rα) or Sol(IL-18Rα)ₓ, and, the Sol(IL-1RAcP) or Sol(IL-1RAcP)ₓ, are on the same protein backbone as a fusion protein (these soluble receptors will be named “Sol(IL-18Rα)ₓ-(IL-1RAcP)ₓ” here after). According to this embodiment, the Sol(IL-18Rα) or Sol(IL-18Rα)ₓ subunit is operably linked to the Sol(IL-1RAcP) or Sol(IL-1RAcP)ₓ subunit. The term “operably linked” indicates that the subunits are associated through peptide linkage, either directly or via a “peptide linker” (as defined here above). In this manner, the fusion protein can be produced recombinantly, by direct expression in a host cell of a nucleic acid molecule encoding the same. The Sol(IL-18Rα) or Sol(IL-18Rα)ₓ subunit can be located upstream (closer to the N-terminus of the protein) or downstream (closer to the C-terminus of the protein) to the Sol(IL-1RAcP) or Sol(IL-1RAcP)ₓ subunit. The subunits are linked either directly or via a “peptide linker”. In a particular embodiment, the fusion protein comprises one Sol(IL-18Rα) subunit and one Sol(IL-1RAcP) subunit as defined herein.

3.3.2 Soluble IL-18Rα Comprising at Least One IL-18Rα Subunit (Sol(IL-18Rα) or Sol(IL-18Rα)ₓ) a and at Least One IL-1RAcP Subunit (Sol(IL-1RAcP) or Sol(IL-1RAcP)ₓ) on the Same Protein Backbone (Sol(IL-18Rα)ₓ-(IL-1RAcP)ₓ) as Fusion Protein:

In yet another particular aspect, the fusion protein comprising, the Sol(IL-18Rα) or Sol(IL-18Rα)ₓ, and, the Sol(IL-1RAcP) or Sol(IL-1RAcP)ₓ, subunits (Sol(IL-18Rα)ₓ-(IL-1RAcP)ₓ) is itself “operably linked” to an additional amino acid domain. The term “operably linked” indicates that the additional amino acid domain is associated through peptide linkage, either directly or via a “peptide linker” as defined here above. In this manner, this fusion protein can be produced recombinantly, by direct expression in a host cell of a nucleic acid molecule encoding the same. The additional amino acid domain may be located upstream (N-ter) or downstream (C-ter) to Sol(IL-18Rα)ₓ-(IL-1RAcP)ₓ. In this embodiment, the additional amino acid domain comprises any functional region providing for instance an increased stability, targeting or bioavailability of the fusion protein; facilitating purification or production, or conferring on the molecule additional biological activity. Specific examples of such additional amino acid sequences include a GST sequence, a His tag sequence, the constant region of an immunoglobulin molecule or a heterodimeric protein hormone such as human chorionic gonadotropin (hCG) as described in U.S. Pat. No. 6,193,972. Also, if needed, the additional amino acid sequence included in the fusion proteins may be eliminated, either at the end of the production/purification process or in vivo, e.g., by means of an appropriate endo-/exopeptidase. For example, a spacer sequence included in the fusion protein may comprise a recognition site for an endopeptidase (such as a caspase) that can be used to separate by enzymatic cleavage the desired polypeptide variant from the additional amino acid domain, either in vivo or in vitro. In a particular aspect of this embodiment, Sol(IL-18Rα)ₓ-(IL-1RAcP)ₓ comprises one Sol(IL-18Rα) subunit and one Sol(IL-1RAcP) subunit as defined here above.

3.3.3 Multimers of Sol(IL-18Rα)ₓ-(IL-1RAcP)ₓ:

In a particular aspect, Sol(IL-18Rα)ₓ-(IL-1RAcP)ₓ soluble receptors are produced as multimers. Each subunit of the multimer comprising one Sol(IL-18Rα)ₓ-(IL-1RAcP)ₓ. These multimers may be homodimeric, heterodimeric, or multimeric soluble receptors, with multimeric receptors generally not comprising more than 9 subunits, preferably not comprising more than 6 subunits, even more preferably not more than 3 subunits and most preferably not comprising more than 2 subunits. Preferably, these multimers soluble receptors are homodimers of Sol(IL-18Rα)ₓ-(IL-1RAcP)ₓ as defined here above. In an embodiment, the subunits of the multimers are linked via covalent linkages. The subunits may be covalently linked by any suitable means, such as via a cross-linking reagent or a polypeptide linker. In another embodiment, the subunits are linked via non-covalent linkages.

In one embodiment, each Sol(IL-18Rα)ₓ-(IL-1RAcP)ₓ subunit is operably linked to an additional amino acid domain that provides for the multimerization of the subunits (in particular the additional domains comprise any functional region providing for dimerization of the subunits). The term “operably linked” is as defined here above. The additional amino acid domain may be located upstream (N-ter) or downstream (C-ter) from the sequence of the Sol(IL-18Rα)ₓ-(IL-1RAcP)ₓ subunit. In this manner, the fusion protein can be produced recombinantly, by direct expression in a host cell of a nucleic acid molecule encoding the same. In these embodiments, soluble IL-18Rα receptors of the invention are multimers of fusion proteins containing a Sol(IL-18Rα)ₓ-(IL-1RAcP)ₓ subunit, operably linked to a multimerizing component capable of interacting with the multimerizing component present in another fusion protein to form a higher order structure, such as a dimer. This type of fusion proteins may be prepared by operably linking the Sol(IL-18Rα)ₓ-(IL-1RAcP)ₓ subunit sequence to domains isolated from other proteins allowing the formation of dimers, trimers, etc. Examples for protein sequences allowing the multimerization of the IL-18Rα soluble receptors of the invention are domains isolated from proteins such as immunoglobulins, hCG (WO 97/30161), collagen X (WO 04/33486), C4BP (WO 04/20639), Erb proteins (WO 98/02540), or coiled coil peptides (WO 01/00814).

In a particular aspect, the multimers are dimers of Sol(IL-18Rα)ₓ-(IL-1RAcP)ₓ where the subunits are operably linked to an immunoglobulin. The term “operably linked” is as defined here above. In this embodiment, the subunits are operably linked to all or a portion of an immunoglobulin, particularly a human immunoglobulin, even more particularly the Fc portion of a human immunoglobulin. Typically an Fc portion of a human immunoglobulin contains two constant region domains (the CH2 and CH3 domains) and a hinge region but lacks the variable region (See e.g. U.S. Pat. Nos. 6,018,026 and 5,750,375). The immunoglobulin may be selected from any of the major classes of immunoglobulins, including IgA, IgD, IgE, IgG and IgM, and any subclass or isotype, e.g. IgG1, IgG2, IgG3 and IgG4; IgA-1 and IgA-2. In an embodiment, the Fc moiety is of human IgG4, which is stable in solution and has little or no complement activating activity. In another embodiment, the Fc moiety is of human IgG1. The Fc part may be mutated in order to prevent unwanted activities, such as complement binding, binding to Fc receptors, or the like. Usually the Sol(IL-18Rα)ₓ-(IL-1RAcP)ₓ subunits are operably linked to the same immunoglobulin (particularly to the Fc portion of a human immunoglobulin, for example of a human IgG4 or human IgG1). The amino acid sequence derived from the immunoglobulin may be linked to the C-terminus or to the N-terminus of Sol(IL-18Rα)ₓ-(IL-1RAcP)ₓ, preferably to the C-terminus. Such fusion proteins can be prepared by transfecting cells with DNA encoding Sol(IL-18Rα)ₓ-(IL-1RAcP)ₓ:Fc fusion protein and/or DNA encoding another Sol(IL-18Rα)ₓ-(IL-1RAcP)ₓ:Fc fusion protein and expressing the dimers in the same cells. In a particular embodiment, the subunits Sol(IL-18Rα)ₓ-(IL-1RAcP)ₓ are the same on each monomer (i.e the dimer is a homodimer of Sol(IL-18Rα)ₓ-(IL-1RAcP)ₓ). Even more particularly, the subunits of Sol(IL-18Rα)ₓ-(IL-1RAcP)ₓ are operably linked to the same immunoglobulin (particularly to the Fc portion of a human immunoglobulin, for example of a human IgG4 or human IgG1). Such fusion proteins can be prepared by transfecting cells with DNA encoding Sol(IL-18Rα)ₓ-(IL-1RAcP)ₓ:Fc fusion protein and expressing the dimers in the same cells. Subunits advantageously comprise a native or heterologous signal peptide when initially synthesized, to promote secretion from the cell, but the signal sequence is cleaved upon secretion. Methods for making immunoglobulin fusion proteins are well known in the art, such as the ones described in Hollenbaugh and Aruffo (“Construction of Immunoglobulin Fusion Proteins”, in Current Protocols in Immunology, Suppl. 4, pages 10.19.1-10.19.11, 1992) or WO 01/03737, for example.

Alternatively, the dimers of Sol(IL-18Rα)ₓ-(IL-1RAcP)ₓ of the present invention can be prepared by operably linking one of the receptor subunit to the constant region of an immunoglobulin heavy chain and operably linking the other receptor subunit to the constant region of an immunoglobulin light chain. The term “operably linked” indicates that Sol(IL-18Rα)ₓ-(IL-1RAcP)ₓ, and the immunoglobulin are associated through peptide linkage, either directly or via a “peptide linker” (as defined here above). For example, a Sol(IL-18Rα)ₓ-(IL-1RAcP)ₓ subunit can be operably linked to the CH₁-hinge-CH2-CH3 region of human IgG1 and another or the same Sol(IL-18Rα)ₓ-(IL-1RAcP)ₓ subunit can be operably linked to the C kappa region of the Ig kappa light chain. The amino acid sequence derived from the immunoglobulin may be linked to the C-terminus or to the N-terminus of the Sol(IL-18Rα)ₓ-(IL-1RAcP)ₓ subunits, preferably to the C-terminus. Cells transfected with DNA encoding the immunoglobulin light chain fusion protein and the immunoglobulin heavy chain fusion protein express heavy chain/light chain heterodimers containing each a Sol(IL-18Rα)ₓ-(IL-1RAcP)ₓ subunit. Both subunits advantageously comprise a native or heterologous signal peptide when initially synthesized, to promote secretion from the cell, but the signal sequence is cleaved upon secretion. In a particular embodiment, the subunits Sol(IL-18Rα)ₓ-(IL-1RAcP)ₓ are the same on each monomer (i.e the dimer is a homodimer of Sol(IL-18Rα)ₓ-(IL-1RAcP)ₓ).

In another particular aspect of the present invention, the subunits of the multimers Sol(IL-18Rα)ₓ-(IL-1RAcP)ₓ (as defined here above) are linked via non-covalent linkages. Non-covalent bonding of the subunits may be achieved by any suitable means that does not interfere with its biological activity (i.e. its ability to reduce the symptoms of MS). In a particular aspect, these multimers are dimers of Sol(IL-18Rα)ₓ-(IL-1RAcP)ₓ where one subunit of Sol(IL-18Rα)ₓ-(IL-1RAcP)ₓ is operably linked to a first compound and another or the same subunit Sol(IL-18Rα)ₓ-(IL-1RAcP)ₓ is operably linked to a second compound that will non-covalently bond to the first compound. The term “operably linked” is as defined here above. Examples of such compounds are biotin and avidin. The dimers of Sol(IL-18Rα)ₓ-(IL-1RAcP)ₓ can be prepared by operably linking one of the receptor subunit to biotin and operably linking the other subunit to avidin. The receptor is thus formed through the non-covalent interactions of biotin with avidin. Other examples include subunits of heterodimeric proteinaceous hormone. In these embodiments, a DNA construct encoding one subunit of Sol(IL-18Rα)ₓ-(IL-1RAcP)ₓ is fused to a DNA construct encoding a subunit of a heterodimeric proteinaceous hormone, such as hCG, and a DNA construct encoding the other Sol(IL-18Rα)ₓ-(IL-1RAcP)ₓ subunit is fused to DNA encoding the other subunit of the heterodimeric proteinaceous hormone, such as hCG (as disclosed in U.S. Pat. No. 6,193,972). These DNA constructs are coexpressed in the same cells leading to the expression of an Sol(IL-18Rα)ₓ-(IL-1RAcP)ₓ heterodimeric receptor fusion protein, as each coexpressed sequence contains a corresponding hormone subunit so as to form a heterodimer upon expression. The amino acid sequence derived from the heterodimeric proteinaceous hormone may be linked to the C-terminus or to the N-terminus of the Sol(IL-18Rα)ₓ-(IL-1RAcP)ₓ subunits, preferably to the C-terminus. Both subunits advantageously comprise a native or heterologous signal peptide when initially synthesized, to promote secretion from the cell, but the signal sequence is cleaved upon secretion. In a particular embodiment, the subunits Sol(IL-18Rα)ₓ-(IL-1RAcP)ₓ are the same on each monomer (i.e the dimer is a homodimer of Sol(IL-18Rα)ₓ-(IL-1RAcP)ₓ).

Other examples for protein sequences allowing the dimerization of the Sol(IL-18Rα)ₓ-(IL-1RAcP)ₓ subunits are domains isolated from proteins such as collagen X (WO 04/33486), C4BP (WO 04/20639), Erb proteins (WO 98/02540), or coiled coil peptides (WO 01/00814).

Also, if needed, fusion proteins described herein may comprise any functional region facilitating purification or production. Specific examples of such additional amino acid sequences include a GST sequence or a His tag sequence.

3.3.4 Soluble IL-18Rα Comprising at Least One IL-18Rα Subunit (Sol(IL-18Rα) or Sol(IL-18Rα)ₓ) and at Least One IL-1RAcP Subunit (Sol(IL-1RAcP) or Sol(IL-1RAcP)ₓ) as Heteromultimers:

In a particular aspect, soluble receptors of the present invention comprising at least one IL-18Rα subunit (Sol(IL-18Rα) or Sol(IL-18Rα)ₓ) and at least one IL-1RAcP subunit (Sol(IL-1RAcP) or Sol(IL-1RAcP)ₓ) are heteromultimers. Each subunit of the heteromultimer comprising:

at least one IL-18Rα subunit (Sol(IL-18Rα) or Sol(IL-18Rα)ₓ) or;

at least one IL-1RAcP subunit (Sol(IL-1RAcP) or Sol(IL-1RAcP)ₓ).

These heteromultimers generally do not comprise more than 9 subunits, preferably not more than 6 subunits, even more preferably not more than 3 subunits and most preferably not more than 2 subunits. Preferably, these heteromultimers soluble receptors are heterodimers comprising one subunit consisting of Sol(IL-18Rα) or Sol(IL-18Rα)ₓ (as defined above) and one subunit consisting of Sol(IL-1RAcP) or Sol(IL-1RAcP)ₓ (as defined above). In an embodiment, the subunits of the heteromultimers are linked via covalent linkages. The subunits may be covalently linked by any suitable means, such as via a cross-linking reagent. In another embodiment, the subunits are linked via non-covalent linkages.

In one embodiment, each subunit of the heteromultimer is operably linked to an additional amino acid domain that provides for the multimerization of the subunits (in particular the additional domains may comprise any functional region providing for dimerization of the subunits). The term “operably linked” is as defined here above. The additional amino acid domain may be located upstream (N-ter) or downstream (C-ter) (preferably downstream (C-ter)) from the sequence of the Sol(IL-18Rα) or Sol(IL-18Rα)ₓ subunit(s) and upstream (N-ter) or downstream (C-ter) (preferably downstream (C-ter)) from the sequence of the Sol(IL-1RAcP) or Sol(IL-1RAcP)ₓ subunit(s). In this manner, the fusion protein can be produced recombinantly, by direct expression in a host cell of a nucleic acid molecule encoding the same. In these embodiments, soluble IL-18Rα receptors of the invention are heteromultimers of fusion proteins containing one subunit consisting of Sol(IL-18Rα) or Sol(IL-18Rα)ₓ or of Sol(IL-1RAcP) or Sol(IL-1RAcP)ₓ, operably linked to a multimerizing component capable of interacting with the multimerizing component present in another fusion protein to form a higher order structure, such as a dimer. This type of fusion proteins may be prepared by operably linking the Sol(IL-18Rα) or Sol(IL-18Rα)ₓ subunit sequence and the Sol(IL-1RAcP) or Sol(IL-1RAcP)ₓ subunit sequence to domains isolated from other proteins allowing the formation of dimers, trimers, etc. Examples for protein sequences allowing the multimerization of the IL-18Rα soluble receptors of the invention are domains isolated from proteins such as immunoglobulins, hCG (WO 97/30161), collagen X (WO 04/33486), C4BP (WO 04/20639), Erb proteins (WO 98/02540), or coiled coil peptides (WO 01/00814).

In a particular aspect, the heteromultimers are heterodimers comprising one subunit consisting of Sol(IL-18Rα) and one subunit consisting of Sol(IL-1RAcP), or one subunit consisting of Sol(IL-18Rα)ₓ and one subunit consisting of Sol(IL-1RAcP), or one subunit consisting of Sol(IL-18Rα) and one subunit consisting of Sol(IL-1RAcP)ₓ, or one subunit consisting of Sol(IL-18Rα)ₓ and one subunit consisting of Sol(IL-1RAcP)ₓ. In yet another particular aspect, the two subunits of the heterodimer are operably linked to an immunoglobulin. The term “operably linked” is as defined here above. In these embodiment, the subunits are operably linked to all or a portion of an immunoglobulin, particularly a human immunoglobulin, even more particularly the Fc portion of a human immunoglobulin. Typically an Fc portion of a human immunoglobulin contains two constant region domains (the CH2 and CH3 domains) and a hinge region but lacks the variable region (See e.g. U.S. Pat. Nos. 6,018,026 and 5,750,375). The immunoglobulin may be selected from any of the major classes of immunoglobulins, including IgA, IgD, IgE, IgG and IgM, and any subclass or isotype, e.g. IgG1, IgG2, IgG3 and IgG4; IgA-1 and IgA-2. In an embodiment, the Fc moiety is of human IgG4, which is stable in solution and has little or no complement activating activity. In another embodiment, the Fc moiety is of human IgG1. The Fc part may be mutated in order to prevent unwanted activities, such as complement binding, binding to Fc receptors, or the like. Usually the two subunits are operably linked to the same immunoglobulin (particularly to the Fc portion of a human immunoglobulin, for example of a human IgG4 or human IgG1). The amino acid sequence derived from the immunoglobulin may be linked to the C-terminus or to the N-terminus of the subunit, preferably to the C-terminus. Such fusion proteins can be prepared by transfecting cells with DNA encoding the first subunit:Fc fusion protein and DNA encoding the other subunit:Fc fusion protein and expressing the dimers in the same cells. Subunits advantageously comprise a native or heterologous signal peptide when initially synthesized, to promote secretion from the cell, but the signal sequence is cleaved upon secretion. Methods for making immunoglobulin fusion proteins are well known in the art, such as the ones described in Hollenbaugh and Aruffo (“Construction of Immunoglobulin Fusion Proteins”, in Current Protocols in Immunology, Suppl. 4, pages 10.19.1-10.19.11, 1992) or WO 01/03737, for example.

Alternatively, the heterodimers comprising one subunit consisting of Sol(IL-18Rα) and one subunit consisting of Sol(IL-1RAcP), or one subunit consisting of Sol(IL-18Rα)ₓ and one subunit consisting of Sol(IL-1RAcP), or one subunit consisting of Sol(IL-18Rα) and one subunit consisting of Sol(IL-1RAcP)ₓ, or one subunit consisting of Sol(IL-18Rα)ₓ and one subunit consisting of Sol(IL-1RAcP)ₓ, of the present invention can be prepared by operably linking one of the receptor subunit to the constant region of an immunoglobulin heavy chain and operably linking the other receptor subunit to the constant region of an immunoglobulin light chain. The term “operably linked” is as defined here above. For example, the Sol(IL-18Rα) or Sol(IL-18Rα)ₓ subunit can be operably linked to the CH₁-hinge-CH2-CH3 region of human IgG1 and the Sol(IL-1RAcP) subunit can be operably linked to the C kappa region of the Ig kappa light chain (or vice versa); or the Sol(IL-18Rα) or Sol(IL-18Rα)ₓ subunit can be operably linked to the CH₁-hinge-CH2-CH3 region of human IgG1 and the Sol(IL-1RAcP)ₓ subunit can be operably linked to the C kappa region of the Ig kappa light chain (or vice versa). The amino acid sequence derived from the immunoglobulin may be linked to the C-terminus or to the N-terminus of the subunits, preferably to the C-terminus. Cells transfected with DNA encoding the immunoglobulin light chain fusion protein and the immunoglobulin heavy chain fusion protein express heavy chain/light chain heterodimers containing each a subunit. Both subunits advantageously comprise a native or heterologous signal peptide when initially synthesized, to promote secretion from the cell, but the signal sequence is cleaved upon secretion.

In another particular aspect of the present invention, the subunits of the heteromultimers are linked via non-covalent linkages. Non-covalent bonding of the subunits may be achieved by any suitable means that does not interfere with its biological activity (i.e. its ability to reduce the symptoms of MS). In a particular aspect, these heteromultimers are heterodimers comprising one subunit consisting of Sol(IL-18Rα) and one subunit consisting of Sol(IL-1RAcP), or one subunit consisting of Sol(IL-18Rα)ₓ and one subunit consisting of Sol(IL-1RAcP), or one subunit consisting of Sol(IL-18Rα) and one subunit consisting of Sol(IL-1RAcP)ₓ, or one subunit consisting of Sol(IL-18Rα)ₓ and one subunit consisting of Sol(IL-1RAcP)ₓ, where one subunit is operably linked to a first compound the other is operably linked to a second compound that will non-covalently bond to the first compound. The term “operably linked” is as defined here above. Examples of such compounds are biotin and avidin. These heterodimers can be prepared by operably linking one of the receptor subunit to biotin and operably linking the other subunit to avidin. The receptor is thus formed through the non-covalent interactions of biotin with avidin. Other examples include subunits of heterodimeric proteinaceous hormone. In these embodiments, a DNA construct encoding one subunit (Sol(IL-18Rα) or Sol(IL-18Rα)ₓ) is fused to a DNA construct encoding a subunit of a heterodimeric proteinaceous hormone, such as hCG, and a DNA construct encoding the other subunit (Sol(IL-1RAcP) or Sol(IL-1RAcP)ₓ) is fused to DNA encoding the other subunit of the heterodimeric proteinaceous hormone, such as hCG (as disclosed in U.S. Pat. No. 6,193,972). These DNA constructs are coexpressed in the same cells leading to the expression of an heterodimeric receptor fusion protein, as each coexpressed sequence contains a corresponding hormone subunit so as to form a heterodimer upon expression. The amino acid sequence derived from the heterodimeric proteinaceous hormone may be linked to the C-terminus or to the N-terminus of the subunits, preferably to the C-terminus. Both subunits advantageously comprise a native or heterologous signal peptide when initially synthesized, to promote secretion from the cell, but the signal sequence is cleaved upon secretion.

Other examples for protein sequences allowing the dimerization of the Sol(IL-18Rα)ₓ-(IL-1RAcP)ₓ subunits are domains isolated from proteins such as collagen X (WO 04/33486), C4BP (WO 04/20639), Erb proteins (WO 98/02540), or coiled coil peptides (WO 01/00814).

In an embodiment, the heteromultimers comprising at least one Sol(IL-18Rα) or Sol(IL-18Rα)ₓ subunit and one Sol(IL-1RAcP) or Sol(IL-1RAcP)ₓ subunit of the present invention are recombinant antibodies. The technology of recombinant antibody is described for example in the U.S. Pat. No. 6,018,026. In that case, the multimer of one Sol(IL-18Rα) or Sol(IL-18Rα)ₓ and Sol(IL-1RAcP) or Sol(IL-1RAcP)ₓ is a multimer polypeptide fusion, comprising: a first Sol(IL-18Rα) or Sol(IL-18Rα)ₓ polypeptide chain and a second Sol(IL-1RAcP) or Sol(IL-1RAcP)ₓ polypeptide chains, wherein one of the polypeptide chain is operably linked to an immunoglobulin heavy chain constant region and the other polypeptide chain is operably linked to an immunoglobulin light chain constant region. In an embodiment, the first Sol(IL-18Rα) or Sol(IL-18Rα)ₓ polypeptide chain is operably linked to an immunoglobulin heavy chain constant region and the second Sol(IL-1RAcP) or Sol(IL-1RAcP)ₓ polypeptide chains is operably linked to an immunoglobulin light chain constant region. In another embodiment, the first Sol(IL-18Rα) or Sol(IL-18Rα)ₓ polypeptide chain is operably linked to an immunoglobulin light chain constant region and the second Sol(IL-1RAcP) or Sol(IL-1RAcP)ₓ polypeptide chains is operably linked to an immunoglobulin heavy chain constant region. The term “operably linked” indicates that Sol(IL-18Rα) or Sol(IL-18Rα)x and Sol(IL-1RAcP) or Sol(IL-1RAcP)ₓ, and the immunoglobulin heavy or light chain constant region are associated through peptide linkage, either directly or via a “peptide linker” (as defined here above). In an embodiment, the immunoglobulin heavy chain constant region domain and the immunoglobulin light chain constant region domain are human immunoglobulin constant regions. In an embodiment, the immunoglobulin heavy chain constant region domain is selected from the group consisting of the constant region of an α, γ, μ, δ or ε human immunoglobulin heavy chain. Preferably, said constant region is the constant region of a γ1, γ2, γ3 or γ4 human immunoglobulin heavy chain. In a preferred embodiment, the immunoglobulin light chain constant region domain is selected from the group consisting of the constant region of a κ or λ human immunoglobulin light chain. The amino acid sequence from the immunoglobulin may be linked to the C-terminus or to the N-terminus of the Sol(IL-18Rα) or Sol(IL-18Rα)ₓ and Sol(IL-1RAcP) or Sol(IL-1RAcP)ₓ subunits, preferably to the C-terminus. Cells transfected with DNA encoding the immunoglobulin light chain fusion protein and the immunoglobulin heavy chain fusion protein express a fusion protein having the structure of an antibody. The resulting protein obtained consists of:

two identical heavy chains constant region operably linked to a Sol(IL-18Rα) or Sol(IL-18Rα)ₓ subunit and two identical light chains constant region operably linked to a Sol(IL-1RAcP) or Sol(IL-1RAcP)ₓ subunit; or

two identical heavy chains constant region operably linked to a Sol(IL-1RAcP) or Sol(IL-1RAcP)ₓ subunit and two identical light chains constant region operably linked to a Sol(IL-18Rα) or Sol(IL-18Rα)ₓ subunit.

As for an antibody, heavy and light chains are disulfide linked (interchain disulfide bond) and heavy chains are disulfide linked (interchain disulfide bond).
The resulting molecule is therefore a homodimer composed of two heterodimers each of these heterodimers being composed of:

an immunoglobulin heavy chain constant region operably linked to a Sol(IL-18Rα) or Sol(IL-18Rα)ₓ polypeptide chain and;

an immunoglobulin light chain constant region operably linked to a Sol(IL-1RAcP) or Sol(IL-1RAcP)ₓ polypeptide chain.

Or a homodimer composed of two heterodimers each of these heterodimers being composed of:

an immunoglobulin heavy chain constant region operably linked to a Sol(IL-1RAcP) or Sol(IL-1RAcP)ₓ polypeptide chain and;

an immunoglobulin light chain constant region operably linked to a Sol(IL-18Rα) or Sol(IL-18Rα)ₓ polypeptide chain.

Both subunits advantageously comprise a native or heterologous signal peptide when initially synthesized, to promote secretion from the cell, but the signal sequence is cleaved upon secretion. In an embodiment, the heavy constant chain is human γ4, which is stable in solution and has little or no complement activating activity. In another embodiment, the heavy constant chain is human γ1. The heavy constant chain may be mutated in order to prevent unwanted activities, such as complement binding, binding to Fc receptors, or the like.

1. In an embodiment the recombinant antibody of the present invention comprises or consists of:

two identical heavy chains constant regions, said heavy chains constant regions being the constant region of γ1, γ2, γ3 or γ4 human immunoglobulin heavy chain, operably linked to the extracellular domain of the human IL-18Rα and;

two identical light chains constant region, said light chain constant region being the constant region of κ or λ human immunoglobulin light chain, operably linked to the extra cellular domain of the human IL-1RAcP. In an embodiment, heavy and light chains are disulfide linked (interchain disulfide bond) and heavy chains are disulfide linked (interchain disulfide bond) as for a natural antibody.

2. In another particular embodiment, the recombinant antibody of the present invention comprises or consists of:

two identical heavy chains constant region, said heavy chains constant region being the constant region of γ1, γ2, γ3 or γ4 human immunoglobulin heavy chain, operably linked to the extracellular domain of the human IL-1RAcP and;

two identical light chains constant region, said light chain constant region being the constant region of κ or λ human immunoglobulin light chain, operably linked to the extra cellular domain of the human IL-18Rα. In an embodiment, heavy and light chains are disulfide linked (interchain disulfide bond) and heavy chains are disulfide linked (interchain disulfide bond) as for a natural antibody.

3. In another embodiment, the present invention resides in a recombinant antibody as defined at point 1 or 2 above wherein the constant regions of the heavy chain are the constant regions of γ1 human immunoglobulin heavy chain.

4. In another embodiment, the present invention resides in a recombinant antibody as defined at point 1, 2 or 3 above wherein the constant regions of the light chain are the constant regions of κ human immunoglobulin light chain.

5. In another embodiment, the present invention resides in a recombinant antibody as defined at point 1, 2, 3 or 4 above wherein the extra cellular domain of the human IL-18Rα consists of amino acids residues 19-329 of SEQ ID NO: 2 or a variant of said polypeptide as defined here above.

6. In another embodiment, the present invention resides in a recombinant antibody as defined at point 1, 2, 3, 4 or 5 above wherein the extra cellular domain of the human IL-1RAcP consists of amino acids residues 21-367 of SEQ ID NO: 6 or a variant of said polypeptide as defined here above.

7. In another embodiment, the present invention resides in a recombinant antibody as defined at point 1, 2, 3, 4, 5 or 6 above wherein the heavy chain constant regions are directly associated through peptide linkage to the extracellular domain of the human IL-18Rα or of the human IL-1RAcP.

8. In another embodiment, the present invention resides in a recombinant antibody as defined at point 1, 2, 3, 4, 5, 6 or 7 above wherein the light chain constant regions are directly associated through peptide linkage to the extracellular domain of the human IL-18Rα or of the human IL-1RAcP.

9. In another embodiment, the present invention resides in a recombinant antibody as defined at point 1, 2, 3, 4, 5 or 6 above wherein the heavy chain constant regions are associated through peptide linkage via a peptide linker to the extracellular domain of the human IL-18Rα or of the human IL-1RAcP. The peptide linker can be as short as 1 to 3 amino acid residues in length (preferably consisting of small amino acids such as glycine, serine, threonine or alanine) or longer, for example 13, 15 or 16 amino acid residues in length, introduced between the subunits. Preferably, the peptide linker is a peptide which is immunologically inert. Said linker may be a tripeptide of the sequence E-F-M (Glu-Phe-Met) (SEQ ID NO: 13), for example, a 13-amino acid linker sequence consisting of Glu-Phe-Gly-Ala-Gly-Leu-Val-Leu-Gly-Gly-Gln-Phe-Met (SEQ ID NO: 14), a 15-amino acid linker sequence consisting of (G₄S)₃ (SEQ ID NO: 15), a 16-amino acid linker sequence consisting of GGSGG SGGGG SGGGG S (SEQ ID NO: 16) or the hinge region of human IgG (e.g. IgG1, IgG2, IgG3 or IgG4). In an embodiment, said peptide linker is a 15-amino acid linker sequence consisting of (G₄S)₃ (SEQ ID NO: 15),

10. In another embodiment, the present invention resides in a recombinant antibody as defined at point 1, 2, 3, 4, 5, 6 or 9 above wherein the light chain constant regions are associated through peptide linkage via a peptide linker to the extracellular domain of the human IL-18Rα or of the human IL-1RAcP. The peptide linker can be as short as 1 to 3 amino acid residues in length (preferably consisting of small amino acids such as glycine, serine, threonine or alanine) or longer, for example 13, 15 or 16 amino acid residues in length, introduced between the subunits. Preferably, the peptide linker is a peptide which is immunologically inert. Said linker may be a tripeptide of the sequence E-F-M (Glu-Phe-Met) (SEQ ID NO: 13), for example, a 13-amino acid linker sequence consisting of Glu-Phe-Gly-Ala-Gly-Leu-Val-Leu-Gly-Gly-Gln-Phe-Met (SEQ ID NO: 14), a 15-amino acid linker sequence consisting of (G₄S)₃ (SEQ ID NO: 15), a 16-amino acid linker sequence consisting of GGSGG SGGGG SGGGG S (SEQ ID NO: 16) or the hinge region of human IgG (e.g. IgG1, IgG2, IgG3 or IgG4). In an embodiment, said peptide linker is a 15-amino acid linker sequence consisting of (G₄S)₃ (SEQ ID NO: 15).

11. In another embodiment, the present invention resides in a recombinant antibody as defined at point 1, 2, 3, 4, 5, 6, 7, 8, 9 or 10 above wherein the heavy constant chain is human γ4, which is stable in solution and has little or no complement activating activity.

12. In another embodiment, the present invention resides in a recombinant antibody as defined at point 1, 2, 3, 4, 5, 6, 7, 8, 9 or 10 above wherein the heavy constant chain is human γ1 and is mutated in order to prevent unwanted activities, such as complement binding, binding to Fc receptors, or the like.

13. In another embodiment, the present invention resides in a recombinant antibody as defined at point 1, 2, 3, 4, 5, 6, 7, 8, 9, 10, 11 or 12 above wherein the heavy chain constant regions are operably linked to the C-terminus or to the N-terminus of the extracellular domain of the human IL-18Rα or of the human IL-1RAcP, preferably to the C-terminus.

14. In another embodiment, the present invention resides in a recombinant antibody as defined at point 1, 2, 3, 4, 5, 6, 7, 8, 9, 10, 11, 12 or 13 above wherein the light chain constant regions are operably linked to the C-terminus or to the N-terminus of the extracellular domain of the human IL-18Rα or of the human IL-1RAcP, preferably to the C-terminus.

15. In another embodiment, the present invention resides in a recombinant antibody as defined at point 1, 2, 3, 4, 5, 6, 7, 8, 9, 10, 11, 12, 13 or 14 above wherein the extracellular domain of the human IL-18Rα or of the human IL-1RAcP is operably linked to the C-terminus or to the N-terminus of the heavy chain constant regions, preferably to the N-terminus.

16. In another embodiment, the present invention resides in a recombinant antibody as defined at point 1, 2, 3, 4, 5, 6, 7, 8, 9, 10, 11, 12, 13, 14 or 15 above wherein the extracellular domain of the human IL-18Rα or of the human IL-1RAcP is operably linked to the C-terminus or to the N-terminus of the light chain constant regions, preferably to the N-terminus.

Also, if needed, fusion proteins described herein may comprise any functional region facilitating purification or production. Specific examples of such additional amino acid sequences include a GST sequence or a His tag sequence.

4) Soluble IL-18Rα Comprising at Least One IL-18Rα Subunit (Sol(IL-18Rα) and/or Sol(IL-18Rα)ₓ) and One IL-1R-rp2 Subunit (Sol(IL-1R-rp2) and/or Sol(IL-1R-rp2)ₓ):

In a particular aspect of the present invention, the soluble IL-18Rα receptors used to treat, prevent or ameliorate the symptoms of an autoimmune or demyelinating disease, in particular MS are soluble receptors comprising at least one IL-18Rα subunit (Sol(IL-18Rα) and/or Sol(IL-18Rα)ₓ as defined here above), and at least one IL-1R-rp2 subunit, as defined here after. The term “soluble receptor” has been defined above.

IL-1R-rp2 (also named IL1RRP2 in the literature) is a member of the IL-1 receptor family and possesses an extracellular domain comprising three immunoglobulin-like domains (Ig domains). A cDNA encoding human IL-1R-rp2 is presented at SEQ ID NO: 7. This cDNA encodes a 575 amino acids long protein (SEQ ID NO: 8) which includes an extracellular domain of 335 amino acids (residues 1-335 from N- to C-terminus of SEQ ID NO: 8) that includes a signal peptide of 19 amino acids (residues 1-19 of SEQ ID NO: 8); a transmembrane region of 21 amino acids (residues 336-356) and a cytoplasmic domain of 219 amino acids (residues 357-575).

4.1 IL-1R-rp2 Subunit and Variants thereof (Named here after “Sol(IL-1R-rp2)”):

In one aspect, the IL-1R-rp2 subunit of the soluble IL-18Rα receptor of the present invention is a polypeptide comprising all or part of the extracellular domain of IL-1R-rp2, in particular all or part of the extracellular domain of human IL-1R-rp2 or a variant thereof.

In an aspect, the IL-1R-rp2 subunit of the soluble IL-18Rα receptor of the present invention (Sol(IL-1R-rp2)) is a polypeptide comprising or consisting of amino acids residues 20-335 of SEQ ID NO: 8, or a variant of said polypeptide. Ordinarily, the variant polypeptides are at least 280 amino acids in length, often at least 300 amino acids in length, more often at least 316 amino acids in length. A variant is defined as a polypeptide having at least 80% amino acid sequence identity with the sequence of reference (here residues 20-335 of SEQ ID NO: 8), preferably at least 90% amino acid sequence identity, more preferably at least 95% amino acid sequence identity, more preferably at least 98% amino acid sequence identity and most preferably at least 99% amino acid sequence identity. More preferably, the variants are differing from the sequence of reference (residues 20-335 of SEQ ID NO: 8) by five, more preferably by four, even more preferably by three, even more preferably by two and most preferably by one amino acid. In some particular aspects of the invention, the variants are differing from the sequence of reference (residues 20-335 of SEQ ID NO: 8) by the lack of 20, 15, 10, 9, 8, 7, 6, 5, 4, 3, 2 or 1 amino acid(s) at the N-terminal and/or C-terminal end. One of skill in the art using the genetic code can readily determine polynucleotides that encode such polypeptides. “Percent (%) amino acid sequence identity” is defined as here above.

In another embodiment, Sol(IL-1R-rp2) is a polypeptide comprising or consisting of amino acids residues 20-221, or 112-335, or 20-125 and 212-335 linked by a peptide bond, of SEQ ID NO: 8, or a variant of said polypeptide. Ordinarily, the variant polypeptides are at least 180 amino acids in length, often at least 202 amino acids in length, often at least 224 amino acids in length, more often at least 230 amino acids in length. A variant is defined as a polypeptide having at least 80% amino acid sequence identity with the sequence of reference (here residues 20-221, or 112-335, or 20-125 and 212-335 linked by a peptide bond, of SEQ ID NO: 8), preferably at least 90% amino acid sequence identity, more preferably at least 95% amino acid sequence identity, more preferably at least 98% amino acid sequence identity and most preferably at least 99% amino acid sequence identity. More preferably, the variants are differing from the sequence of reference (here residues 20-221, or 112-335, or 20-125 and 212-335 linked by a peptide bond, of SEQ ID NO: 8), by five, more preferably by four, even more preferably by three, even more preferably by two and most preferably by one amino acid. In some particular aspects of the invention, the variants are differing from the sequence of reference (here residues 20-221, or 112-335, or 20-125 and 212-335 linked by a peptide bond, of SEQ ID NO: 8), by the lack of 20, 15, 10, 9, 8, 7, 6, 5, 4, 3, 2 or 1 amino acid(s) at the N-terminal and/or C-terminal end. One of skill in the art using the genetic code can readily determine polynucleotides that encode such polypeptides. “Percent (%) amino acid sequence identity” is defined as here above.

In yet another embodiment, Sol(IL-1R-rp2) is a polypeptide comprising or consisting of amino acids residues 20-125, or 112-221, or 212-335 of SEQ ID NO: 8, or a variant of said polypeptide. Ordinarily, the variant polypeptides are at least 95 amino acids in length, often at least 106 amino acids in length, often at least 110 amino acids in length, more often at least 124 amino acids in length. A variant is defined as a polypeptide having at least 80% amino acid sequence identity with the sequence of reference (here residues 20-125, or 112-221, or 212-335 of SEQ ID NO: 8), preferably at least 90% amino acid sequence identity, more preferably at least 95% amino acid sequence identity, more preferably at least 98% amino acid sequence identity and most preferably at least 99% amino acid sequence identity. More preferably, the variants are differing from the sequence of reference (here residues 20-125, or 112-221, or 212-335 of SEQ ID NO: 8) by five, more preferably by four, even more preferably by three, even more preferably by two and most preferably by one amino acid. In some particular aspects of the invention, the variants are differing from the sequence of reference (here residues 20-125, or 112-221, or 212-335 of SEQ ID NO: 8), by the lack of 20, 15, 10, 9, 8, 7, 6, 5, 4, 3, 2 or 1 amino acid(s) at the N-terminal and/or C-terminal end. One of skill in the art using the genetic code can readily determine polynucleotides that encode such polypeptides. “Percent (%) amino acid sequence identity” is defined as here above.

4.2 Soluble IL-1R-rp2 Comprising at Least Two IL-1R-rp2 Subunits or Variant thereof on the Same Protein Backbone (Named here after “Sol(IL-1R-rp2).”):

As it will be described here after, the present invention, among other aspects, pertains to soluble IL-18Rα receptors comprising at least two IL-1R-rp2 subunits (at least two Sol(IL-1R-rp2)). These soluble IL-1R-rp2 comprising at least two IL-1R-rp2 subunits (i.e at least two Sol(IL-1R-rp2) subunits as defined here above) are on the same protein backbone as a fusion protein and are named here after “Sol(IL-1R-rp2)ₓ”. In a particular embodiment, the fusion protein comprises two Sol(IL-1R-rp2) subunits. In yet another particular embodiment, the at least two Sol(IL-1R-rp2) subunits are the same (i.e the fusion protein is a homomultimer of Sol(IL-1R-rp2)), and in a particular embodiment the fusion protein is a homodimer of Sol(IL-1R-rp2).

The at least two IL-1R-rp2 subunits are operably linked to one another. The term “operably linked” indicates that the subunits are associated through peptide linkage, either directly or via a “peptide linker”. In this manner, the fusion protein can be produced recombinantly, by direct expression in a host cell of a nucleic acid molecule encoding the same. The subunits are linked either directly or via a “peptide linker”. The peptide linker can be as short as 1 to 3 amino acid residues in length (preferably consisting of small amino acids such as glycine, serine, threonine or alanine) or longer, for example 13, 15 or 16 amino acid residues in length, introduced between the subunits. Preferably, the peptide linker is a peptide which is immunologically inert. Said linker may be a tripeptide of the sequence E-F-M (Glu-Phe-Met) (SEQ ID NO: 13), for example, a 13-amino acid linker sequence consisting of Glu-Phe-Gly-Ala-Gly-Leu-Val-Leu-Gly-Gly-Gln-Phe-Met (SEQ ID NO: 14), a 15-amino acid linker sequence consisting of (G₄S)₃ (SEQ ID NO: 15), a 16-amino acid linker sequence consisting of GGSGG SGGGG SGGGG S (SEQ ID NO: 16) or the hinge region of human IgG (e.g. IgG1, IgG2, IgG3 or IgG4).

4.3 Soluble IL-18Rα Comprising at Least One IL-18Rα Subunit (Sol(IL-18Rα) or Sol(IL-18Rα)ₓ) and at Least One IL-1R-rp2 Subunit (Sol(IL-1R-rp2) or Sol(IL-1R-rp2)ₓ):

As disclosed here above, the present invention, among other aspects, pertains to soluble IL-18Rα receptors comprising at least one IL-18Rα subunit ((Sol(IL-18Rα) or Sol(IL-18Rα)ₓ as defined here above), and one IL-1R-rp2 subunit (Sol(IL-1R-rp2) or Sol(IL-1R-rp2)ₓ as defined here above).

4.3.1 Soluble IL-18Rα Comprising at Least One IL-18Rα Subunit (Sol(IL-18Rα) or Sol(IL-18Rα)ₓ) and at Least One IL-1R-rp2 Subunit (Sol(IL-1R-rp2) or Sol(IL-1R-rp2)ₓ) on the Same Protein Backbone (Named here after “Sol(IL-18Rα)ₓ-(IL-1R-rp2)ₓ”):

In one aspect of the present invention, the Sol(IL-18Rα) or Sol(IL-18Rα)ₓ, and, the Sol(IL-1R-rp2) or Sol(IL-1R-rp2)ₓ, are on the same protein backbone as a fusion protein (these soluble receptors will be named “Sol(IL-18Rα)ₓ-(IL-1R-rp2)ₓ” here after). According to this embodiment, the Sol(IL-18Rα) or Sol(IL-18Rα)ₓ subunit is operably linked to the Sol(IL-1R-rp2) or Sol(IL-1R-rp2)ₓ subunit. The term “operably linked” indicates that the subunits are associated through peptide linkage, either directly or via a “peptide linker” (as defined here above). In this manner, the fusion protein can be produced recombinantly, by direct expression in a host cell of a nucleic acid molecule encoding the same. The Sol(IL-18Rα) or Sol(IL-18Rα)ₓ subunit can be located upstream (closer to the N-terminus of the protein) or downstream (closer to the C-terminus of the protein) to the Sol(IL-1R-rp2) or Sol(IL-1R-rp2)ₓ subunit. The subunits are linked either directly or via a “peptide linker”. In a particular embodiment, the fusion protein comprises one Sol(IL-18Rα) subunit and one Sol(IL-1R-rp2) subunit as defined herein.

4.3.2 Soluble IL-18Rα Comprising at Least One IL-18Rα Subunit (Sol(IL-18Rα) or Sol(IL-18Rα)ₓ) a and at Least One IL-1R-rp2 Subunit (Sol(IL-1R-rp2) or Sol(IL-1R-rp2)ₓ) on the same protein backbone (Sol(IL-18Rα)ₓ-(IL-1R-rp2)ₓ) as fusion protein:

In yet another particular aspect, the fusion protein comprising, the Sol(IL-18Rα) or Sol(IL-18Rα)ₓ, and, the Sol(IL-1R-rp2) or Sol(IL-1R-rp2)ₓ, subunits (Sol(IL-18Rα)ₓ-(IL-1R-rp2)ₓ) is itself “operably linked” to an additional amino acid domain. The term “operably linked” indicates that the additional amino acid domain is associated through peptide linkage, either directly or via a “peptide linker” as defined here above. In this manner, this fusion protein can be produced recombinantly, by direct expression in a host cell of a nucleic acid molecule encoding the same. The additional amino acid domain may be located upstream (N-ter) or downstream (C-ter) to Sol(IL-18Rα)ₓ-(IL-1R-rp2)ₓ. In this embodiment, the additional amino acid domain comprises any functional region providing for instance an increased stability, targeting or bioavailability of the fusion protein; facilitating purification or production, or conferring on the molecule additional biological activity. Specific examples of such additional amino acid sequences include a GST sequence, a His tag sequence, the constant region of an immunoglobulin molecule or a heterodimeric protein hormone such as human chorionic gonadotropin (hCG) as described in U.S. Pat. No. 6,193,972. Also, if needed, the additional amino acid sequence included in the fusion proteins may be eliminated, either at the end of the production/purification process or in vivo, e.g., by means of an appropriate endo-/exopeptidase. For example, a spacer sequence included in the fusion protein may comprise a recognition site for an endopeptidase (such as a caspase) that can be used to separate by enzymatic cleavage the desired polypeptide variant from the additional amino acid domain, either in vivo or in vitro. In a particular aspect of this embodiment, Sol(IL-18Rα)ₓ-(IL-1R-rp2)ₓ comprises one Sol(IL-18Rα) subunit and one Sol(IL-1R-rp2) subunit as defined here above.

4.3.3 Multimers of Sol(IL-18Rα)ₓ-(IL-1R-rp2)ₓ:

In a particular aspect, Sol(IL-18Rα)ₓ-(IL-1R-rp2)ₓ soluble receptors are produced as multimers. Each subunit of the multimer comprising one Sol(IL-18Rα)ₓ-(IL-1R-rp2)ₓ. These multimers may be homodimeric, heterodimeric, or multimeric soluble receptors, with multimeric receptors generally not comprising more than 9 subunits, preferably not comprising more than 6 subunits, even more preferably not more than 3 subunits and most preferably not comprising more than 2 subunits. Preferably, these multimers soluble receptors are homodimers of Sol(IL-18Rα)ₓ-(IL-1R-rp²)ₓ as defined here above. In an embodiment, the subunits of the multimers are linked via covalent linkages. The subunits may be covalently linked by any suitable means, such as via a cross-linking reagent or a polypeptide linker. In another embodiment, the subunits are linked via non-covalent linkages.

In one embodiment, each Sol(IL-18Rα)ₓ-(IL-1R-rp2)ₓ subunit is operably linked to an additional amino acid domain that provides for the multimerization of the subunits (in particular the additional domains comprise any functional region providing for dimerization of the subunits). The term “operably linked” is as defined here above. The additional amino acid domain may be located upstream (N-ter) or downstream (C-ter) from the sequence of the Sol(IL-18Rα)ₓ-(IL-1R-rp2)ₓ subunit. In this manner, the fusion protein can be produced recombinantly, by direct expression in a host cell of a nucleic acid molecule encoding the same. In these embodiments, soluble IL-18Rα receptors of the invention are multimers of fusion proteins containing a Sol(IL-18Rα)ₓ-(IL-1R-rp2)ₓ subunit, operably linked to a multimerizing component capable of interacting with the multimerizing component present in another fusion protein to form a higher order structure, such as a dimer. This type of fusion proteins may be prepared by operably linking the Sol(IL-18Rα)ₓ-(IL-1R-rp2)ₓ subunit sequence to domains isolated from other proteins allowing the formation of dimers, trimers, etc. Examples for protein sequences allowing the multimerization of the IL-18Rα soluble receptors of the invention are domains isolated from proteins such as immunoglobulins, hCG (WO 97/30161), collagen X (WO 04/33486), C4BP (WO 04/20639), Erb proteins (WO 98/02540), or coiled coil peptides (WO 01/00814).

In a particular aspect, the multimers are dimers of Sol(IL-18Rα)ₓ-(IL-1R-rp2)ₓ where the subunits are operably linked to an immunoglobulin. The term “operably linked” is as defined here above. In this embodiment, the subunits are operably linked to all or a portion of an immunoglobulin, particularly a human immunoglobulin, even more particularly the Fc portion of a human immunoglobulin. Typically an Fc portion of a human immunoglobulin contains two constant region domains (the CH2 and CH3 domains) and a hinge region but lacks the variable region (See e.g. U.S. Pat. Nos. 6,018,026 and 5,750,375). The immunoglobulin may be selected from any of the major classes of immunoglobulins, including IgA, IgD, IgE, IgG and IgM, and any subclass or isotype, e.g. IgG1, IgG2, IgG3 and IgG4; IgA-1 and IgA-2. In an embodiment, the Fc moiety is of human IgG4, which is stable in solution and has little or no complement activating activity. In another embodiment, the Fc moiety is of human IgG1. The Fc part may be mutated in order to prevent unwanted activities, such as complement binding, binding to Fc receptors, or the like. Usually the Sol(IL-18Rα)ₓ-(IL-1R-rp2)ₓ subunits are operably linked to the same immunoglobulin (particularly to the Fc portion of a human immunoglobulin, for example of a human IgG4 or human IgG1). The amino acid sequence derived from the immunoglobulin may be linked to the C-terminus or to the N-terminus of Sol(IL-18Rα)ₓ-(IL-1R-rp2)ₓ, preferably to the C-terminus. Such fusion proteins can be prepared by transfecting cells with DNA encoding Sol(IL-18Rα)ₓ-(IL-IR-rp2)ₓ:Fc fusion protein and/or DNA encoding another Sol(IL-18Rα)ₓ-(IL-1R-rp2)ₓ:Fc fusion protein and expressing the dimers in the same cells. In a particular embodiment, the subunits Sol(IL-18Rα)ₓ-(IL-1R-rp2)ₓ are the same on each monomer (i.e the dimer is a homodimer of Sol(IL-18Rα)ₓ-(IL-1R-rp2)ₓ). Even more particularly, the subunits of Sol(IL-18Rα)ₓ-(IL-1R-rp2)ₓ are operably linked to the same immunoglobulin (particularly to the Fc portion of a human immunoglobulin, for example of a human IgG4 or human IgG1). Such fusion proteins can be prepared by transfecting cells with DNA encoding Sol(IL-18Rα)ₓ-(IL-1R-rp2)ₓ:Fc fusion protein and expressing the dimers in the same cells. Subunits advantageously comprise a native or heterologous signal peptide when initially synthesized, to promote secretion from the cell, but the signal sequence is cleaved upon secretion. Methods for making immunoglobulin fusion proteins are well known in the art, such as the ones described in Hollenbaugh and Aruffo (“Construction of Immunoglobulin Fusion Proteins”, in Current Protocols in Immunology, Suppl. 4, pages 10.19.1-10.19.11, 1992) or WO 01/03737, for example.

Alternatively, the dimers of Sol(IL-18Rα)ₓ-(IL-1R-rp2)ₓ of the present invention can be prepared by operably linking one of the receptor subunit to the constant region of an immunoglobulin heavy chain and operably linking the other receptor subunit to the constant region of an immunoglobulin light chain. The term “operably linked” indicates that Sol(IL-18Rα)ₓ-(IL-1R-rp2)ₓ, and the immunoglobulin are associated through peptide linkage, either directly or via a “peptide linker” (as defined here above). For example, a Sol(IL-18Rα)ₓ-(IL-1R-rp2)ₓ subunit can be operably linked to the CH₁-hinge-CH2-CH3 region of human IgG1 and another or the same Sol(IL-18Rα)ₓ-(IL-1R-rp2)ₓ subunit can be operably linked to the C kappa region of the Ig kappa light chain. The amino acid sequence derived from the immunoglobulin may be linked to the C-terminus or to the N-terminus of the Sol(IL-18Rα)ₓ-(IL-1R-rp2)ₓ subunits, preferably to the C-terminus. Cells transfected with DNA encoding the immunoglobulin light chain fusion protein and the immunoglobulin heavy chain fusion protein express heavy chain/light chain heterodimers containing each a Sol(IL-18Rα)ₓ-(IL-1R-rp2)ₓ subunit. Both subunits advantageously comprise a native or heterologous signal peptide when initially synthesized, to promote secretion from the cell, but the signal sequence is cleaved upon secretion. In a particular embodiment, the subunits Sol(IL-18Rα)ₓ-(IL-1R-rp2)ₓ are the same on each monomer (i.e the dimer is a homodimer of Sol(IL-18Rα)ₓ-(IL-1R-rp2)ₓ).

In another particular aspect of the present invention, the subunits of the multimers Sol(IL-18Rα)ₓ-(IL-1R-rp2)ₓ (as defined here above) are linked via non-covalent linkages. Non-covalent bonding of the subunits may be achieved by any suitable means that does not interfere with its biological activity (i.e. its ability to reduce the symptoms of MS). In a particular aspect, these multimers are dimers of Sol(IL-18Rα)ₓ-(IL-1R-rp2)ₓ where one subunit of Sol(IL-18Rα)ₓ-(IL-1R-rp2)ₓ is operably linked to a first compound and another or the same subunit Sol(IL-18Rα)ₓ-(IL-1R-rp2)ₓ is operably linked to a second compound that will non-covalently bond to the first compound. The term “operably linked” is as defined here above. Examples of such compounds are biotin and avidin. The dimers of Sol(IL-18Rα)ₓ-(IL-1R-rp2)ₓ can be prepared by operably linking one of the receptor subunit to biotin and operably linking the other subunit to avidin. The receptor is thus formed through the non-covalent interactions of biotin with avidin. Other examples include subunits of heterodimeric proteinaceous hormone. In these embodiments, a DNA construct encoding one subunit of Sol(IL-18Rα)ₓ-(IL-1R-rp2)ₓ is fused to a DNA construct encoding a subunit of a heterodimeric proteinaceous hormone, such as hCG, and a DNA construct encoding the other Sol(IL-18Rα)ₓ-(IL-1R-rp2)ₓ subunit is fused to DNA encoding the other subunit of the heterodimeric proteinaceous hormone, such as hCG (as disclosed in U.S. Pat. No. 6,193,972). These DNA constructs are coexpressed in the same cells leading to the expression of an Sol(IL-18Rα)ₓ-(IL-1R-rp2)ₓ heterodimeric receptor fusion protein, as each coexpressed sequence contains a corresponding hormone subunit so as to form a heterodimer upon expression. The amino acid sequence derived from the heterodimeric proteinaceous hormone may be linked to the C-terminus or to the N-terminus of the Sol(IL-18Rα)ₓ-(IL-1R-rp2)ₓ subunits, preferably to the C-terminus. Both subunits advantageously comprise a native or heterologous signal peptide when initially synthesized, to promote secretion from the cell, but the signal sequence is cleaved upon secretion. In a particular embodiment, the subunits Sol(IL-18Rα)ₓ-(IL-1R-rp2)ₓ are the same on each monomer (i.e the dimer is a homodimer of Sol(IL-18Rα)ₓ-(IL-1R-rp2)ₓ).

Other examples for protein sequences allowing the dimerization of the Sol(IL-18Rα)ₓ-(IL-1R-rp2)ₓ subunits are domains isolated from proteins such as collagen X (WO 04/33486), C4BP (WO 04/20639), Erb proteins (WO 98/02540), or coiled coil peptides (WO 01/00814).

Also, if needed, fusion proteins described herein may comprise any functional region facilitating purification or production. Specific examples of such additional amino acid sequences include a GST sequence or a His tag sequence.

4.3.4 Soluble IL-18Rα Comprising at Least One IL-18Rα Subunit (Sol(IL-18Rα) or Sol(IL-18Rα)ₓ) and at Least One IL-1R-rp2 Subunit (Sol(IL-1R-rp2) or Sol(IL-1R-rp2)ₓ) as Heteromultimers:

In a particular aspect, soluble receptors of the present invention comprising at least one IL-18Rα subunit (Sol(IL-18Rα) or Sol(IL-18Rα)ₓ) and at least one IL-1R-rp2 subunit (Sol(IL-1R-rp2) or Sol(IL-1R-rp2)ₓ) are heteromultimers. Each subunit of the heteromultimer comprising:

at least one IL-18Rα subunit (Sol(IL-18Rα) or Sol(IL-18Rα)ₓ) or;

at least one IL-1R-rp2 subunit (Sol(IL-1R-rp2) or Sol(IL-1R-rp2)ₓ).

These heteromultimers generally do not comprise more than 9 subunits, preferably not more than 6 subunits, even more preferably not more than 3 subunits and most preferably not more than 2 subunits. Preferably, these heteromultimers soluble receptors are heterodimers comprising one subunit consisting of Sol(IL-18Rα) or Sol(IL-18Rα)ₓ (as defined above) and one subunit consisting of Sol(IL-1R-rp2) or Sol(IL-1R-rp2)ₓ (as defined above). In an embodiment, the subunits of the heteromultimers are linked via covalent linkages. The subunits may be covalently linked by any suitable means, such as via a cross-linking reagent. In another embodiment, the subunits are linked via non-covalent linkages.

In one embodiment, each subunit of the heteromultimer is operably linked to an additional amino acid domain that provides for the multimerization of the subunits (in particular the additional domains may comprise any functional region providing for dimerization of the subunits). The term “operably linked” is as defined here above. The additional amino acid domain may be located upstream (N-ter) or downstream (C-ter) (preferably downstream (C-ter)) from the sequence of the Sol(IL-18Rα) or Sol(IL-18Rα)ₓ subunit(s) and upstream (N-ter) or downstream (C-ter) (preferably downstream (C-ter)) from the sequence of the Sol(IL-1R-rp2) or Sol(IL-1R-rp2)ₓ subunit(s). In this manner, the fusion protein can be produced recombinantly, by direct expression in a host cell of a nucleic acid molecule encoding the same. In these embodiments, soluble IL-18Rα receptors of the invention are heteromultimers of fusion proteins containing one subunit consisting of Sol(IL-18Rα) or Sol(IL-18Rα)ₓ or of Sol(IL-1R-rp2) or Sol(IL-1R-rp2)ₓ, operably linked to a multimerizing component capable of interacting with the multimerizing component present in another fusion protein to form a higher order structure, such as a dimer. This type of fusion proteins may be prepared by operably linking the Sol(IL-18Rα) or Sol(IL-18Rα)ₓ subunit sequence and the Sol(IL-IR-rp2) or Sol(IL-1R-rp2)ₓ subunit sequence to domains isolated from other proteins allowing the formation of dimers, trimers, etc. Examples for protein sequences allowing the multimerization of the IL-18Rα soluble receptors of the invention are domains isolated from proteins such as immunoglobulins, hCG (WO 97/30161), collagen X (WO 04/33486), C4BP (WO 04/20639), Erb proteins (WO 98/02540), or coiled coil peptides (WO 01/00814).

In a particular aspect, the heteromultimers are heterodimers comprising one subunit consisting of Sol(IL-18Rα) and one subunit consisting of Sol(IL-1R-rp2), or one subunit consisting of Sol(IL-18Rα)ₓ and one subunit consisting of Sol(IL-1R-rp2), or one subunit consisting of Sol(IL-18Rα) and one subunit consisting of Sol(IL-1R-rp2)ₓ, or one subunit consisting of Sol(IL-18Rα)ₓ and one subunit consisting of Sol(IL-1R-rp²)ₓ. In yet another particular aspect, the two subunits of the heterodimer are operably linked to an immunoglobulin. The term “operably linked” is as defined here above. In these embodiments, the subunits are operably linked to all or a portion of an immunoglobulin, particularly a human immunoglobulin, even more particularly the Fc portion of a human immunoglobulin. Typically an Fc portion of a human immunoglobulin contains two constant region domains (the CH2 and CH3 domains) and a hinge region but lacks the variable region (See e.g. U.S. Pat. Nos. 6,018,026 and 5,750,375). The immunoglobulin may be selected from any of the major classes of immunoglobulins, including IgA, IgD, IgE, IgG and IgM, and any subclass or isotype, e.g. IgG1, IgG2, IgG3 and IgG4; IgA-1 and IgA-2. In an embodiment, the Fc moiety is of human IgG4, which is stable in solution and has little or no complement activating activity. In another embodiment, the Fc moiety is of human IgG1. The Fc part may be mutated in order to prevent unwanted activities, such as complement binding, binding to Fc receptors, or the like. Usually the two subunits are operably linked to the same immunoglobulin (particularly to the Fc portion of a human immunoglobulin, for example of a human IgG4 or human IgG1). The amino acid sequence derived from the immunoglobulin may be linked to the C-terminus or to the N-terminus of the subunit, preferably to the C-terminus. Such fusion proteins can be prepared by transfecting cells with DNA encoding the first subunit:Fc fusion protein and DNA encoding the other subunit:Fc fusion protein and expressing the dimers in the same cells. Subunits advantageously comprise a native or heterologous signal peptide when initially synthesized, to promote secretion from the cell, but the signal sequence is cleaved upon secretion. Methods for making immunoglobulin fusion proteins are well known in the art, such as the ones described in Hollenbaugh and Aruffo (“Construction of Immunoglobulin Fusion Proteins”, in Current Protocols in Immunology, Suppl. 4, pages 10.19.1-10.19.11, 1992) or WO 01/03737, for example.

Alternatively, the heterodimers comprising one subunit consisting of Sol(IL-18Rα) and one subunit consisting of Sol(IL-1R-rp2), or one subunit consisting of Sol(IL-18Rα)ₓ and one subunit consisting of Sol(IL-1R-rp2), or one subunit consisting of Sol(IL-18Rα) and one subunit consisting of Sol(IL-1R-rp2)ₓ, or one subunit consisting of Sol(IL-18Rα)ₓ and one subunit consisting of Sol(IL-1R-rp2)ₓ, of the present invention can be prepared by operably linking one of the receptor subunit to the constant region of an immunoglobulin heavy chain and operably linking the other receptor subunit to the constant region of an immunoglobulin light chain. The term “operably linked” is as defined here above. For example, the Sol(IL-18Rα) or Sol(IL-18Rα)ₓ subunit can be operably linked to the CH₁-hinge-CH2-CH3 region of human IgG1 and the Sol(IL-1R-rp2) subunit can be operably linked to the C kappa region of the Ig kappa light chain (or vice versa); or the Sol(IL-18Rα) or Sol(IL-18Rα)ₓ subunit can be operably linked to the CH₁-hinge-CH2-CH3 region of human IgG1 and the Sol(IL-1R-rp2)ₓ subunit can be operably linked to the C kappa region of the Ig kappa light chain (or vice versa). The amino acid sequence derived from the immunoglobulin may be linked to the C-terminus or to the N-terminus of the subunits, preferably to the C-terminus. Cells transfected with DNA encoding the immunoglobulin light chain fusion protein and the immunoglobulin heavy chain fusion protein express heavy chain/light chain heterodimers containing each a subunit. Both subunits advantageously comprise a native or heterologous signal peptide when initially synthesized, to promote secretion from the cell, but the signal sequence is cleaved upon secretion.

In another particular aspect of the present invention, the subunits of the heteromultimers are linked via non-covalent linkages. Non-covalent bonding of the subunits may be achieved by any suitable means that does not interfere with its biological activity (i.e. its ability to reduce the symptoms of MS). In a particular aspect, these heteromultimers are heterodimers comprising one subunit consisting of Sol(IL-18Rα) and one subunit consisting of Sol(IL-1R-rp2), or one subunit consisting of Sol(IL-18Rα)ₓ and one subunit consisting of Sol(IL-1R-rp2), or one subunit consisting of Sol(IL-18Rα) and one subunit consisting of Sol(IL-1R-rp2)ₓ, or one subunit consisting of Sol(IL-18Rα)ₓ and one subunit consisting of Sol(IL-1R-rp2)ₓ, where one subunit is operably linked to a first compound the other is operably linked to a second compound that will non-covalently bond to the first compound. The term “operably linked” is as defined here above. Examples of such compounds are biotin and avidin. These heterodimers can be prepared by operably linking one of the receptor subunit to biotin and operably linking the other subunit to avidin. The receptor is thus formed through the non-covalent interactions of biotin with avidin. Other examples include subunits of heterodimeric proteinaceous hormone. In these embodiments, a DNA construct encoding one subunit (Sol(IL-18Rα) or Sol(IL-18Rα)ₓ) is fused to a DNA construct encoding a subunit of a heterodimeric proteinaceous hormone, such as hCG, and a DNA construct encoding the other subunit (Sol(IL-1R-rp2) or Sol(IL-1R-rp2)ₓ) is fused to DNA encoding the other subunit of the heterodimeric proteinaceous hormone, such as hCG (as disclosed in U.S. Pat. No. 6,193,972). These DNA constructs are coexpressed in the same cells leading to the expression of an heterodimeric receptor fusion protein, as each coexpressed sequence contains a corresponding hormone subunit so as to form a heterodimer upon expression. The amino acid sequence derived from the heterodimeric proteinaceous hormone may be linked to the C-terminus or to the N-terminus of the subunits, preferably to the C-terminus. Both subunits advantageously comprise a native or heterologous signal peptide when initially synthesized, to promote secretion from the cell, but the signal sequence is cleaved upon secretion.

Other examples for protein sequences allowing the dimerization of the Sol(IL-18Rα)ₓ-(IL-1R-rp2)ₓ subunits are domains isolated from proteins such as collagen X (WO 04/33486), C4BP (WO 04/20639), Erb proteins (WO 98/02540), or coiled coil peptides (WO 01/00814).

In an embodiment, the heteromultimers comprising at least one Sol(IL-18Rα) or Sol(IL-18Rα)ₓ subunit and one Sol(IL-1R-rp2) or Sol(IL-1R-rp2)ₓ subunit of the present invention are recombinant antibodies. The technology of recombinant antibody is described for example in the U.S. Pat. No. 6,018,026. In that case, the multimer of one Sol(IL-18Rα) or Sol(IL-18Rα)ₓ and Sol(IL-1R-rp2) or Sol(IL-1R-rp2)ₓ is a multimer polypeptide fusion, comprising: a first Sol(IL-18Rα) or Sol(IL-18Rα)ₓ polypeptide chain and a second Sol(IL-1R-rp2) or Sol(IL-1R-rp2)ₓ polypeptide chains, wherein one of the polypeptide chain is operably linked to an immunoglobulin heavy chain constant region and the other polypeptide chain is operably linked to an immunoglobulin light chain constant region. In an embodiment, the first Sol(IL-18Rα) or Sol(IL-18Rα)ₓ polypeptide chain is operably linked to an immunoglobulin heavy chain constant region and the second Sol(IL-1R-rp2) or Sol(IL-1R-rp2)ₓ polypeptide chains is operably linked to an immunoglobulin light chain constant region. In another embodiment, the first Sol(IL-18Rα) or Sol(IL-18Rα)ₓ polypeptide chain is operably linked to an immunoglobulin light chain constant region and the second Sol(IL-1R-rp2) or Sol(IL-1R-rp2)ₓ polypeptide chains is operably linked to an immunoglobulin heavy chain constant region. The term “operably linked” indicates that Sol(IL-18Rα) or Sol(IL-18Rα)x and Sol(IL-1R-rp2) or Sol(IL-1R-rp2)ₓ, and the immunoglobulin heavy or light chain constant region are associated through peptide linkage, either directly or via a “peptide linker” (as defined here above). In an embodiment, the immunoglobulin heavy chain constant region domain and the immunoglobulin light chain constant region domain are human immunoglobulin constant regions. In an embodiment, the immunoglobulin heavy chain constant region domain is selected from the group consisting of the constant region of an α, γ, μ, δ or ε human immunoglobulin heavy chain. Preferably, said constant region is the constant region of a γ1, γ2, γ3 or γ4 human immunoglobulin heavy chain. In a preferred embodiment, the immunoglobulin light chain constant region domain is selected from the group consisting of the constant region of a κ or λ human immunoglobulin light chain. The amino acid sequence from the immunoglobulin may be linked to the C-terminus or to the N-terminus of the Sol(IL-18Rα) or Sol(IL-18Rα)ₓ and Sol(IL-1R-rp2) or Sol(IL-1R-rp2)ₓ subunits, preferably to the C-terminus. Cells transfected with DNA encoding the immunoglobulin light chain fusion protein and the immunoglobulin heavy chain fusion protein express a fusion protein having the structure of an antibody. The resulting protein obtained consists of:

two identical heavy chains constant region operably linked to a Sol(IL-18Rα) or Sol(IL-18Rα)ₓ subunit and two identical light chains constant region operably linked to a Sol(IL-1R-rp2) or Sol(IL-1R-rp2)ₓ subunit; or

two identical heavy chains constant region operably linked to a Sol(IL-1R-rp2) or Sol(IL-1R-rp2)ₓ subunit and two identical light chains constant region operably linked to a Sol(IL-18Rα) or Sol(IL-18Rα)ₓ subunit.

As for an antibody, heavy and light chains are disulfide linked (interchain disulfide bond) and heavy chains are disulfide linked (interchain disulfide bond).
The resulting molecule is therefore a homodimer composed of two heterodimers each of these heterodimers being composed of:

an immunoglobulin heavy chain constant region operably linked to a Sol(IL-18Rα) or Sol(IL-18Rα)ₓ polypeptide chain and;

an immunoglobulin light chain constant region operably linked to a Sol(IL-1R-rp2) or Sol(IL-1R-rp2)ₓ polypeptide chain.
Or a homodimer composed of two heterodimers each of these heterodimers being composed of:

an immunoglobulin heavy chain constant region operably linked to a Sol(IL-1R-rp2) or Sol(IL-1R-rp2)ₓ polypeptide chain and;

an immunoglobulin light chain constant region operably linked to a Sol(IL-18Rα) or Sol(IL-18Rα)ₓ polypeptide chain.
Both subunits advantageously comprise a native or heterologous signal peptide when initially synthesized, to promote secretion from the cell, but the signal sequence is cleaved upon secretion. In an embodiment, the heavy constant chain is human γ4, which is stable in solution and has little or no complement activating activity. In another embodiment, the heavy constant chain is human γ1. The heavy constant chain may be mutated in order to prevent unwanted activities, such as complement binding, binding to Fc receptors, or the like.

1. In an embodiment the recombinant antibody of the present invention comprises or consists of:

two identical heavy chains constant regions, said heavy chains constant regions being the constant region of γ1, γ2, γ3 or γ4 human immunoglobulin heavy chain, operably linked to the extracellular domain of the human IL-18Rα and;

two identical light chains constant region, said light chain constant region being the constant region of κ or λ human immunoglobulin light chain, operably linked to the extra cellular domain of the human IL-1R-rp2. In an embodiment, heavy and light chains are disulfide linked (interchain disulfide bond) and heavy chains are disulfide linked (interchain disulfide bond) as for a natural antibody.

2. In another particular embodiment, the recombinant antibody of the present invention comprises or consists of:

two identical heavy chains constant region, said heavy chains constant region being the constant region of γ1, γ2, γ3 or γ4 human immunoglobulin heavy chain, operably linked to the extracellular domain of the human IL-1R-rp2 and;

two identical light chains constant region, said light chain constant region being the constant region of κ or λ human immunoglobulin light chain, operably linked to the extra cellular domain of the human IL-18Rα. In an embodiment, heavy and light chains are disulfide linked (interchain disulfide bond) and heavy chains are disulfide linked (interchain disulfide bond) as for a natural antibody.

3. In another embodiment, the present invention resides in a recombinant antibody as defined at point 1 or 2 above wherein the constant regions of the heavy chain are the constant regions of γ1 human immunoglobulin heavy chain.

4. In another embodiment, the present invention resides in a recombinant antibody as defined at point 1, 2 or 3 above wherein the constant regions of the light chain are the constant regions of κ human immunoglobulin light chain.

5. In another embodiment, the present invention resides in a recombinant antibody as defined at point 1, 2, 3 or 4 above wherein the extra cellular domain of the human IL-18Rα consists of amino acids residues 19-329 of SEQ ID NO: 2 or a variant of said polypeptide as defined here above.

6. In another embodiment, the present invention resides in a recombinant antibody as defined at point 1, 2, 3, 4 or 5 above wherein the extra cellular domain of the human IL-1R-rp2 consists of amino acids residues 20-335 of SEQ ID NO: 8 or a variant of said polypeptide as defined here above.

7. In another embodiment, the present invention resides in a recombinant antibody as defined at point 1, 2, 3, 4, 5 or 6 above wherein the heavy chain constant regions are directly associated through peptide linkage to the extracellular domain of the human IL-18Rα or of the human IL-1R-rp2.

8. In another embodiment, the present invention resides in a recombinant antibody as defined at point 1, 2, 3, 4, 5, 6 or 7 above wherein the light chain constant regions are directly associated through peptide linkage to the extracellular domain of the human IL-18Rα or of the human IL-1R-rp2.

9. In another embodiment, the present invention resides in a recombinant antibody as defined at point 1, 2, 3, 4, 5 or 6 above wherein the heavy chain constant regions are associated through peptide linkage via a peptide linker to the extracellular domain of the human IL-18Rα or of the human IL-1R-rp2. The peptide linker can be as short as 1 to 3 amino acid residues in length (preferably consisting of small amino acids such as glycine, serine, threonine or alanine) or longer, for example 13, 15 or 16 amino acid residues in length, introduced between the subunits. Preferably, the peptide linker is a peptide which is immunologically inert. Said linker may be a tripeptide of the sequence E-F-M (Glu-Phe-Met) (SEQ ID NO: 13), for example, a 13-amino acid linker sequence consisting of Glu-Phe-Gly-Ala-Gly-Leu-Val-Leu-Gly-Gly-Gln-Phe-Met (SEQ ID NO: 14), a 15-amino acid linker sequence consisting of (G₄S)₃ (SEQ ID NO: 15), a 16-amino acid linker sequence consisting of GGSGG SGGGG SGGGG S (SEQ ID NO: 16) or the hinge region of human IgG (e.g. IgG1, IgG2, IgG3 or IgG4). In an embodiment, said peptide linker is a 15-amino acid linker sequence consisting of (G₄S)₃ (SEQ ID NO: 15),

10. In another embodiment, the present invention resides in a recombinant antibody as defined at point 1, 2, 3, 4, 5, 6 or 9 above wherein the light chain constant regions are associated through peptide linkage via a peptide linker to the extracellular domain of the human IL-18Rα or of the human IL-1R-rp2. The peptide linker can be as short as 1 to 3 amino acid residues in length (preferably consisting of small amino acids such as glycine, serine, threonine or alanine) or longer, for example 13, 15 or 16 amino acid residues in length, introduced between the subunits. Preferably, the peptide linker is a peptide which is immunologically inert. Said linker may be a tripeptide of the sequence E-F-M (Glu-Phe-Met) (SEQ ID NO: 13), for example, a 13-amino acid linker sequence consisting of Glu-Phe-Gly-Ala-Gly-Leu-Val-Leu-Gly-Gly-Gln-Phe-Met (SEQ ID NO: 14), a 15-amino acid linker sequence consisting of (G₄S)₃ (SEQ ID NO: 15), a 16-amino acid linker sequence consisting of GGSGG SGGGG SGGGG S (SEQ ID NO: 16) or the hinge region of human IgG (e.g. IgG1, IgG2, IgG3 or IgG4). In an embodiment, said peptide linker is a 15-amino acid linker sequence consisting of (G₄S)₃ (SEQ ID NO: 15).

11. In another embodiment, the present invention resides in a recombinant antibody as defined at point 1, 2, 3, 4, 5, 6, 7, 8, 9 or 10 above wherein the heavy constant chain is human γ4, which is stable in solution and has little or no complement activating activity.

12. In another embodiment, the present invention resides in a recombinant antibody as defined at point 1, 2, 3, 4, 5, 6, 7, 8, 9 or 10 above wherein the heavy constant chain is human γ1 and is mutated in order to prevent unwanted activities, such as complement binding, binding to Fc receptors, or the like.

13. In another embodiment, the present invention resides in a recombinant antibody as defined at point 1, 2, 3, 4, 5, 6, 7, 8, 9, 10, 11 or 12 above wherein the heavy chain constant regions are operably linked to the C-terminus or to the N-terminus of the extracellular domain of the human IL-18Rα or of the human IL-1R-rp2, preferably to the C-terminus.

14. In another embodiment, the present invention resides in a recombinant antibody as defined at point 1, 2, 3, 4, 5, 6, 7, 8, 9, 10, 11, 12 or 13 above wherein the light chain constant regions are operably linked to the C-terminus or to the N-terminus of the extracellular domain of the human IL-18Rα or of the human IL-1R-rp2, preferably to the C-terminus.

15. In another embodiment, the present invention resides in a recombinant antibody as defined at point 1, 2, 3, 4, 5, 6, 7, 8, 9, 10, 11, 12, 13 or 14 above wherein the extracellular domain of the human IL-18Rα or of the human IL-1R-rp2 is operably linked to the C-terminus or to the N-terminus of the heavy chain constant regions, preferably to the N-terminus.

16. In another embodiment, the present invention resides in a recombinant antibody as defined at point 1, 2, 3, 4, 5, 6, 7, 8, 9, 10, 11, 12, 13, 14 or 15 above wherein the extracellular domain of the human IL-18Rα or of the human IL-1R-rp2 is operably linked to the C-terminus or to the N-terminus of the light chain constant regions, preferably to the N-terminus.

Also, if needed, fusion proteins described herein may comprise any functional region facilitating purification or production. Specific examples of such additional amino acid sequences include a GST sequence or a His tag sequence.

5) Soluble IL-18Rα Comprising at Least One IL-18Rα Subunit (Sol(IL-18Rα) and/or Sol(IL-18Rα)ₓ) and One T1/ST2 Subunit (Sol(T1/ST2) and/or Sol(T1/ST2)ₓ):

In a particular aspect of the present invention, the soluble IL-18Rα receptors used to treat, prevent or ameliorate the symptoms of an autoimmune or demyelinating disease, in particular MS, are soluble receptors comprising at least one IL-18Rα subunit (Sol(IL-18Rα) and/or Sol(IL-18Rα)ₓ as defined here above), and at least one T1/ST2 subunit, as defined here after. The term “soluble receptor” has been defined above.

T1/ST2 (also named DER4, FIT-1, MGC32623, ST2L or ST2V in the literature) is a member of the IL-1 receptor family and possesses an extracellular domain comprising three immunoglobulin-like domains (Ig domains). A cDNA encoding human T1/ST2 is presented at SEQ ID NO: 9. This cDNA encodes a 556 amino acids long protein (SEQ ID NO: 10) which includes an extracellular domain of 328 amino acids (residues 1-328 from N- to C-terminus of SEQ ID NO: 10) that includes a signal peptide of 18 amino acids (residues 1-18 of SEQ ID NO: 10); a transmembrane region of 21 amino acids (residues 329-349) and a cytoplasmic domain of 207 amino acids (residues 350-556).

5.1 T1/ST2 Subunit and Variants thereof (Named here after “Sol(T1/ST2)”):

In one aspect, the T1/ST2 subunit of the soluble IL-18Rα receptor of the present invention is a polypeptide comprising all or part of the extracellular domain of T1/ST2, in particular all or part of the extracellular domain of human T1/ST2 or a variant thereof.

In an aspect, the T1/ST2 subunit of the soluble IL-18Rα receptor of the present invention (Sol(T1/ST2)) is a polypeptide comprising or consisting of amino acids residues 19-328 of SEQ ID NO: 10, or a variant of said polypeptide. Ordinarily, the variant polypeptides are at least 280 amino acids in length, often at least 300 amino acids in length, more often at least 310 amino acids in length. A variant is defined as a polypeptide having at least 80% amino acid sequence identity with the sequence of reference (here residues 19-328 of SEQ ID NO: 10), preferably at least 90% amino acid sequence identity, more preferably at least 95% amino acid sequence identity, more preferably at least 98% amino acid sequence identity and most preferably at least 99% amino acid sequence identity. More preferably, the variants are differing from the sequence of reference (here residues 19-328 of SEQ ID NO: 10) by five, more preferably by four, even more preferably by three, even more preferably by two and most preferably by one amino acid. In some particular aspects of the invention, the variants are differing from the sequence of reference (here residues 19-328 of SEQ ID NO: 10) by the lack of 20, 15, 10, 9, 8, 7, 6, 5, 4, 3, 2 or 1 amino acid(s) at the N-terminal and/or C-terminal end. One of skill in the art using the genetic code can readily determine polynucleotides that encode such polypeptides. “Percent (%) amino acid sequence identity” is defined as here above.

In another embodiment, Sol(T1/ST2) is a polypeptide comprising or consisting of amino acids residues 19-211, or 104-328, or 19-113 and 198-328 linked by a peptide bond, of SEQ ID NO: 10, or a variant of said polypeptide. Ordinarily, the variant polypeptides are at least 180 amino acids in length, often at least 193 amino acids in length, often at least 225 amino acids in length, more often at least 226 amino acids in length. A variant is defined as a polypeptide having at least 80% amino acid sequence identity with the sequence of reference (here residues 19-211, or 104-328, or 19-113 and 198-328 linked by a peptide bond, of SEQ ID NO: 10), preferably at least 90% amino acid sequence identity, more preferably at least 95% amino acid sequence identity, more preferably at least 98% amino acid sequence identity and most preferably at least 99% amino acid sequence identity. More preferably, the variants are differing from the sequence of reference (here residues 19-211, or 104-328, or 19-113 and 198-328 linked by a peptide bond, of SEQ ID NO: 10), by five, more preferably by four, even more preferably by three, even more preferably by two and most preferably by one amino acid. In some particular aspects of the invention, the variants are differing from the sequence of reference (here residues 19-211, or 104-328, or 19-113 and 198-328 linked by a peptide bond, of SEQ ID NO: 10), by the lack of 20, 15, 10, 9, 8, 7, 6, 5, 4, 3, 2 or 1 amino acid(s) at the N-terminal and/or C-terminal end. One of skill in the art using the genetic code can readily determine polynucleotides that encode such polypeptides. “Percent (%) amino acid sequence identity” is defined as here above.

In yet another embodiment, Sol(T1/ST2) is a polypeptide comprising or consisting of amino acids residues 19-113, or 104-211, or 198-328 of SEQ ID NO: 10, or a variant of said polypeptide. Ordinarily, the variant polypeptides are at least 85 amino acids in length, often at least 95 amino acids in length, often at least 108 amino acids in length, more often at least 131 amino acids in length. A variant is defined as a polypeptide having at least 80% amino acid sequence identity with the sequence of reference (here residues 19-113, or 104-211, or 198-328 of SEQ ID NO: 10), preferably at least 90% amino acid sequence identity, more preferably at least 95% amino acid sequence identity, more preferably at least 98% amino acid sequence identity and most preferably at least 99% amino acid sequence identity. More preferably, the variants are differing from the sequence of reference (here residues 19-113, or 104-211, or 198-328 of SEQ ID NO: 10) by five, more preferably by four, even more preferably by three, even more preferably by two and most preferably by one amino acid. In some particular aspects of the invention, the variants are differing from the sequence of reference (here residues 19-113, or 104-211, or 198-328 of SEQ ID NO: 10), by the lack of 20, 15, 10, 9, 8, 7, 6, 5, 4, 3, 2 or 1 amino acid(s) at the N-terminal and/or C-terminal end. One of skill in the art using the genetic code can readily determine polynucleotides that encode such polypeptides. “Percent (%) amino acid sequence identity” is defined as here above.

5.2 Soluble T1/ST2 Comprising at Least Two T1/ST2 Subunits or Variant thereof on the Same Protein Backbone (Named here after “Sol(T1/ST2)ₓ”):

As it will be described here after, the present invention, among other aspects, pertains to soluble IL-18Rα receptors comprising at least two T1/ST2 subunits (at least two Sol(T1/ST2)). These soluble T1/ST2 comprising at least two T1/ST2 subunits (i.e at least two Sol(T1/ST2) subunits as defined here above) are on the same protein backbone as a fusion protein and are named here after “Sol(T1/ST2)ₓ”. In a particular embodiment, the fusion protein comprises two Sol(T1/ST2) subunits. In yet another particular embodiment, the at least two Sol(T1/ST2) subunits are the same (i.e the fusion protein is a homomultimer of Sol(T1/ST2)), and in a particular embodiment the fusion protein is a homodimer of Sol(T1/ST2).

The at least two T1/ST2 subunits are operably linked to one another. The term “operably linked” indicates that the subunits are associated through peptide linkage, either directly or via a “peptide linker”. In this manner, the fusion protein can be produced recombinantly, by direct expression in a host cell of a nucleic acid molecule encoding the same. The subunits are linked either directly or via a “peptide linker”. The peptide linker can be as short as 1 to 3 amino acid residues in length (preferably consisting of small amino acids such as glycine, serine, threonine or alanine) or longer, for example 13, 15 or 16 amino acid residues in length, introduced between the subunits. Preferably, the peptide linker is a peptide which is immunologically inert. Said linker may be a tripeptide of the sequence E-F-M (Glu-Phe-Met) (SEQ ID NO: 13), for example, a 13-amino acid linker sequence consisting of Glu-Phe-Gly-Ala-Gly-Leu-Val-Leu-Gly-Gly-Gln-Phe-Met (SEQ ID NO: 14), a 15-amino acid linker sequence consisting of (G₄S)₃ (SEQ ID NO: 15), a 16-amino acid linker sequence consisting of GGSGG SGGGG SGGGG S (SEQ ID NO: 16) or the hinge region of human IgG (e.g. IgG1, IgG2, IgG3 or IgG4).

5.3 Soluble IL-18Rα Comprising at Least One IL-18Rα Subunit (Sol(IL-18Rα) or Sol(IL-18Rα)ₓ) and at Least One T1/ST2 Subunit (Sol(T1/ST2) or Sol(T1/ST2)ₓ):

As disclosed here above, the present invention, among other aspects, pertains to soluble IL-18Rα receptors comprising at least one IL-18Rα subunit ((Sol(IL-18Rα) or Sol(IL-18Rα)ₓ as defined here above), and one T1/ST2 subunit (Sol(T1/ST2) or Sol(T1/ST2)ₓ as defined here above).

5.3.1 Soluble IL-18Rα Comprising at Least One IL-18Rα Subunit (Sol(IL-18Rα) or Sol(IL-18Rα)ₓ) and at Least One T1/ST2 Subunit (Sol(T1/ST2) or Sol(T1/ST2)ₓ) on the Same Protein Backbone (Named here after “Sol(IL-18Rα)ₓ-(T1/ST2)ₓ”):

In one aspect of the present invention, the Sol(IL-18Rα) or Sol(IL-18Rα)ₓ, and, the Sol(T1/ST2) or Sol(T1/ST2)ₓ, are on the same protein backbone as a fusion protein (these soluble receptors will be named “Sol(IL-18Rα)ₓ-(T1/ST2)ₓ” here after). According to this embodiment, the Sol(IL-18Rα) or Sol(IL-18Rα)ₓ subunit is operably linked to the Sol(T1/ST2) or Sol(T1/ST2)ₓ subunit. The term “operably linked” indicates that the subunits are associated through peptide linkage, either directly or via a “peptide linker” (as defined here above). In this manner, the fusion protein can be produced recombinantly, by direct expression in a host cell of a nucleic acid molecule encoding the same. The Sol(IL-18Rα) or Sol(IL-18Rα)ₓ subunit can be located upstream (closer to the N-terminus of the protein) or downstream (closer to the C-terminus of the protein) to the Sol(T1/ST2) or Sol(T1/ST2)ₓ subunit. The subunits are linked either directly or via a “peptide linker”. In a particular embodiment, the fusion protein comprises one Sol(IL-18Rα) subunit and one Sol(T1/ST2) subunit as defined herein.

5.3.2 Soluble IL-18Rα Comprising at Least One IL-18Rα Subunit (Sol(IL-18Rα) or Sol(IL-18Rα)ₓ) a and at Least One T1/ST2 Subunit (Sol(T1/ST2) or Sol(T1/ST2)ₓ) on the Same Protein Backbone (Sol(IL-18Rα)ₓ-(T1/ST2)ₓ) as Fusion Protein:

In yet another particular aspect, the fusion protein comprising, the Sol(IL-18Rα) or Sol(IL-18Rα)ₓ, and, the Sol(T1/ST2) or Sol(T1/ST2)ₓ, subunits (Sol(IL-18Rα)ₓ-(T1/ST2)ₓ) is itself “operably linked” to an additional amino acid domain. The term “operably linked” indicates that the additional amino acid domain is associated through peptide linkage, either directly or via a “peptide linker” as defined here above. In this manner, this fusion protein can be produced recombinantly, by direct expression in a host cell of a nucleic acid molecule encoding the same. The additional amino acid domain may be located upstream (N-ter) or downstream (C-ter) to Sol(IL-18Rα)ₓ-(T1/ST2)ₓ. In this embodiment, the additional amino acid domain comprises any functional region providing for instance an increased stability, targeting or bioavailability of the fusion protein; facilitating purification or production, or conferring on the molecule additional biological activity. Specific examples of such additional amino acid sequences include a GST sequence, a His tag sequence, the constant region of an immunoglobulin molecule or a heterodimeric protein hormone such as human chorionic gonadotropin (hCG) as described in U.S. Pat. No. 6,193,972. Also, if needed, the additional amino acid sequence included in the fusion proteins may be eliminated, either at the end of the production/purification process or in vivo, e.g., by means of an appropriate endo-/exopeptidase. For example, a spacer sequence included in the fusion protein may comprise a recognition site for an endopeptidase (such as a caspase) that can be used to separate by enzymatic cleavage the desired polypeptide variant from the additional amino acid domain, either in vivo or in vitro. In a particular aspect of this embodiment, Sol(IL-18Rα)ₓ-(T1/ST2)ₓ comprises one Sol(IL-18Rα) subunit and one Sol(T1/ST2) subunit as defined here above.

5.3.3 Multimers of Sol(IL-18Rα)ₓ-(T1/ST2)ₓ:

In a particular aspect, Sol(IL-18Rα)ₓ-(T 1/ST2)ₓ soluble receptors are produced as multimers. Each subunit of the multimer comprising one Sol(IL-18Rα)ₓ-(T1/ST2)ₓ. These multimers may be homodimeric, heterodimeric, or multimeric soluble receptors, with multimeric receptors generally not comprising more than 9 subunits, preferably not comprising more than 6 subunits, even more preferably not more than 3 subunits and most preferably not comprising more than 2 subunits. Preferably, these multimers soluble receptors are homodimers of Sol(IL-18Rα)ₓ-(T1/ST2)ₓ as defined here above. In an embodiment, the subunits of the multimers are linked via covalent linkages. The subunits may be covalently linked by any suitable means, such as via a cross-linking reagent or a polypeptide linker. In another embodiment, the subunits are linked via non-covalent linkages.

In one embodiment, each Sol(IL-18Rα)ₓ-(T1/ST2)ₓ subunit is operably linked to an additional amino acid domain that provides for the multimerization of the subunits (in particular the additional domains comprise any functional region providing for dimerization of the subunits). The term “operably linked” is as defined here above. The additional amino acid domain may be located upstream (N-ter) or downstream (C-ter) from the sequence of the Sol(IL-18Rα)ₓ-(T1/ST2)ₓ subunit. In this manner, the fusion protein can be produced recombinantly, by direct expression in a host cell of a nucleic acid molecule encoding the same. In these embodiments, soluble IL-18Rα receptors of the invention are multimers of fusion proteins containing a Sol(IL-18Rα)ₓ-(T1/ST2)ₓ subunit, operably linked to a multimerizing component capable of interacting with the multimerizing component present in another fusion protein to form a higher order structure, such as a dimer. This type of fusion proteins may be prepared by operably linking the Sol(IL-18Rα)ₓ-(T1/ST2)ₓ subunit sequence to domains isolated from other proteins allowing the formation of dimers, trimers, etc. Examples for protein sequences allowing the multimerization of the IL-18Rα soluble receptors of the invention are domains isolated from proteins such as immunoglobulins, hCG (WO 97/30161), collagen X (WO 04/33486), C4BP (WO 04/20639), Erb proteins (WO 98/02540), or coiled coil peptides (WO 01/00814).

In a particular aspect, the multimers are dimers of Sol(IL-18Rα)ₓ-(T1/ST2)ₓ where the subunits are operably linked to an immunoglobulin. The term “operably linked” is as defined here above. In this embodiment, the subunits are operably linked to all or a portion of an immunoglobulin, particularly a human immunoglobulin, even more particularly the Fc portion of a human immunoglobulin. Typically an Fc portion of a human immunoglobulin contains two constant region domains (the CH2 and CH3 domains) and a hinge region but lacks the variable region (See e.g. U.S. Pat. Nos. 6,018,026 and 5,750,375). The immunoglobulin may be selected from any of the major classes of immunoglobulins, including IgA, IgD, IgE, IgG and IgM, and any subclass or isotype, e.g. IgG1, IgG2, IgG3 and IgG4; IgA-1 and IgA-2. In an embodiment, the Fc moiety is of human IgG4, which is stable in solution and has little or no complement activating activity. In another embodiment, the Fc moiety is of human IgG1. The Fc part may be mutated in order to prevent unwanted activities, such as complement binding, binding to Fc receptors, or the like. Usually the Sol(IL-18Rα)ₓ-(T1/ST2)ₓ subunits are operably linked to the same immunoglobulin (particularly to the Fc portion of a human immunoglobulin, for example of a human IgG4 or human IgG1). The amino acid sequence derived from the immunoglobulin may be linked to the C-terminus or to the N-terminus of Sol(IL-18Rα)ₓ-(T1/ST2)ₓ, preferably to the C-terminus. Such fusion proteins can be prepared by transfecting cells with DNA encoding Sol(IL-18Rα)ₓ-(T1/ST2)ₓ:Fc fusion protein and/or DNA encoding another Sol(IL-18Rα)ₓ-(T1/ST2)ₓ:Fc fusion protein and expressing the dimers in the same cells. In a particular embodiment, the subunits Sol(IL-18Rα)ₓ-(T1/ST2)ₓ are the same on each monomer (i.e the dimer is a homodimer of Sol(IL-18Rα)ₓ-(T1/ST2)ₓ). Even more particularly, the subunits of Sol(IL-18Rα)ₓ-(T1/ST2)ₓ are operably linked to the same immunoglobulin (particularly to the Fc portion of a human immunoglobulin, for example of a human IgG4 or human IgG1). Such fusion proteins can be prepared by transfecting cells with DNA encoding Sol(IL-18Rα)ₓ-(T1/ST2)ₓ:Fc fusion protein and expressing the dimers in the same cells. Subunits advantageously comprise a native or heterologous signal peptide when initially synthesized, to promote secretion from the cell, but the signal sequence is cleaved upon secretion. Methods for making immunoglobulin fusion proteins are well known in the art, such as the ones described in Hollenbaugh and Aruffo (“Construction of Immunoglobulin Fusion Proteins”, in Current Protocols in Immunology, Suppl. 4, pages 10.19.1-10.19.11, 1992) or WO 01/03737, for example.

Alternatively, the dimers of Sol(IL-18Rα)ₓ-(T1/ST2)ₓ of the present invention can be prepared by operably linking one of the receptor subunit to the constant region of an immunoglobulin heavy chain and operably linking the other receptor subunit to the constant region of an immunoglobulin light chain. The term “operably linked” indicates that Sol(IL-18Rα)ₓ-(T1/ST2)ₓ, and the immunoglobulin are associated through peptide linkage, either directly or via a “peptide linker” (as defined here above). For example, a Sol(IL-18Rα)ₓ-(T1/ST2)ₓ subunit can be operably linked to the CH₁-hinge-CH2-CH3 region of human IgG1 and another or the same Sol(IL-18Rα)ₓ-(T1/ST2)ₓ subunit can be operably linked to the C kappa region of the Ig kappa light chain. The amino acid sequence derived from the immunoglobulin may be linked to the C-terminus or to the N-terminus of the Sol(IL-18Rα)ₓ-(T1/ST2)ₓ subunits, preferably to the C-terminus. Cells transfected with DNA encoding the immunoglobulin light chain fusion protein and the immunoglobulin heavy chain fusion protein express heavy chain/light chain heterodimers containing each a Sol(IL-18Rα)ₓ-(T1/ST2)ₓ subunit. Both subunits advantageously comprise a native or heterologous signal peptide when initially synthesized, to promote secretion from the cell, but the signal sequence is cleaved upon secretion. In a particular embodiment, the subunits Sol(IL-18Rα)ₓ-(T1/ST2)ₓ are the same on each monomer (i.e the dimer is a homodimer of Sol(IL-18Rα)ₓ-(T1/ST2)ₓ).

In another particular aspect of the present invention, the subunits of the multimers Sol(IL-18Rα)ₓ-(T 1/ST2)ₓ (as defined here above) are linked via non-covalent linkages. Non-covalent bonding of the subunits may be achieved by any suitable means that does not interfere with its biological activity (i.e. its ability to reduce the symptoms of MS). In a particular aspect, these multimers are dimers of Sol(IL-18Rα)ₓ-(T1/ST2)ₓ where one subunit of Sol(IL-18Rα)ₓ-(T1/ST2)ₓ is operably linked to a first compound and another or the same subunit Sol(IL-18Rα)ₓ-(T1/ST2)ₓ is operably linked to a second compound that will non-covalently bond to the first compound. The term “operably linked” is as defined here above. Examples of such compounds are biotin and avidin. The dimers of Sol(IL-18Rα)ₓ-(T1/ST2)ₓ can be prepared by operably linking one of the receptor subunit to biotin and operably linking the other subunit to avidin. The receptor is thus formed through the non-covalent interactions of biotin with avidin. Other examples include subunits of heterodimeric proteinaceous hormone. In these embodiments, a DNA construct encoding one subunit of Sol(IL-18Rα)ₓ-(T1/ST2)ₓ is fused to a DNA construct encoding a subunit of a heterodimeric proteinaceous hormone, such as hCG, and a DNA construct encoding the other Sol(IL-18Rα)ₓ-(T1/ST2)ₓ subunit is fused to DNA encoding the other subunit of the heterodimeric proteinaceous hormone, such as hCG (as disclosed in U.S. Pat. No. 6,193,972). These DNA constructs are coexpressed in the same cells leading to the expression of an Sol(IL-18Rα)ₓ-(T1/ST2)ₓ heterodimeric receptor fusion protein, as each coexpressed sequence contains a corresponding hormone subunit so as to form a heterodimer upon expression. The amino acid sequence derived from the heterodimeric proteinaceous hormone may be linked to the C-terminus or to the N-terminus of the Sol(IL-18Rα)ₓ-(T1/ST2)ₓ subunits, preferably to the C-terminus. Both subunits advantageously comprise a native or heterologous signal peptide when initially synthesized, to promote secretion from the cell, but the signal sequence is cleaved upon secretion. In a particular embodiment, the subunits Sol(IL-18Rα)ₓ-(T1/ST2)ₓ are the same on each monomer (i.e the dimer is a homodimer of Sol(IL-18Rα)ₓ-(T1/ST2)ₓ).

Other examples for protein sequences allowing the dimerization of the Sol(IL-18Rα)ₓ-(T1/ST2)ₓ subunits are domains isolated from proteins such as collagen X (WO 04/33486), C4BP (WO 04/20639), Erb proteins (WO 98/02540), or coiled coil peptides (WO 01/00814).

Also, if needed, fusion proteins described herein may comprise any functional region facilitating purification or production. Specific examples of such additional amino acid sequences include a GST sequence or a His tag sequence.

5.3.4 Soluble IL-18Rα Comprising at Least One IL-18Rα Subunit (Sol(IL-18Rα) or Sol(IL-18Rα)ₓ) and at Least One T1/ST2 Subunit (Sol(T1/ST2) or Sol(T1/ST2)ₓ) as Heteromultimers:

In a particular aspect, soluble receptors of the present invention comprising at least one IL-18Rα subunit (Sol(IL-18Rα) or Sol(IL-18Rα)ₓ) and at least one T1/ST2 subunit (Sol(T1/ST2) or Sol(T1/ST2)ₓ) are heteromultimers. Each subunit of the heteromultimer comprising:

at least one IL-18Rα subunit (Sol(IL-18Rα) or Sol(IL-18Rα)) or;

at least one T1/ST2 subunit (Sol(T1/ST2) or Sol(T1/ST2)ₓ).

These heteromultimers generally do not comprise more than 9 subunits, preferably not more than 6 subunits, even more preferably not more than 3 subunits and most preferably not more than 2 subunits. Preferably, these heteromultimers soluble receptors are heterodimers comprising one subunit consisting of Sol(IL-18Rα) or Sol(IL-18Rα)ₓ (as defined above) and one subunit consisting of Sol(T1/ST2) or Sol(T1/ST2)ₓ (as defined above). In an embodiment, the subunits of the heteromultimers are linked via covalent linkages. The subunits may be covalently linked by any suitable means, such as via a cross-linking reagent. In another embodiment, the subunits are linked via non-covalent linkages.

In one embodiment, each subunit of the heteromultimer is operably linked to an additional amino acid domain that provides for the multimerization of the subunits (in particular the additional domains may comprise any functional region providing for dimerization of the subunits). The term “operably linked” is as defined here above. The additional amino acid domain may be located upstream (N-ter) or downstream (C-ter) (preferably downstream (C-ter)) from the sequence of the Sol(IL-18Rα) or Sol(IL-18Rα)ₓ subunit(s) and upstream (N-ter) or downstream (C-ter) (preferably downstream (C-ter)) from the sequence of the Sol(T1/ST2) or Sol(T1/ST2)ₓ subunit(s). In this manner, the fusion protein can be produced recombinantly, by direct expression in a host cell of a nucleic acid molecule encoding the same. In these embodiments, soluble IL-18Rα receptors of the invention are heteromultimers of fusion proteins containing one subunit consisting of Sol(IL-18Rα) or Sol(IL-18Rα)ₓ or of Sol(T1/ST2) or Sol(T1/ST2)ₓ, operably linked to a multimerizing component capable of interacting with the multimerizing component present in another fusion protein to form a higher order structure, such as a dimer. This type of fusion proteins may be prepared by operably linking the Sol(IL-18Rα) or Sol(IL-18Rα)ₓ subunit sequence and the Sol(T1/ST2) or Sol(T1/ST2)ₓ subunit sequence to domains isolated from other proteins allowing the formation of dimers, trimers, etc. Examples for protein sequences allowing the multimerization of the IL-18Rα soluble receptors of the invention are domains isolated from proteins such as immunoglobulins, hCG (WO 97/30161), collagen X (WO 04/33486), C4BP (WO 04/20639), Erb proteins (WO 98/02540), or coiled coil peptides (WO 01/00814).

In a particular aspect, the heteromultimers are heterodimers comprising one subunit consisting of Sol(IL-18Rα) and one subunit consisting of Sol(T1/ST2), or one subunit consisting of Sol(IL-18Rα)ₓ and one subunit consisting of Sol(T1/ST2), or one subunit consisting of Sol(IL-18Rα) and one subunit consisting of Sol(T1/ST2)ₓ, or one subunit consisting of Sol(IL-18Rα)ₓ and one subunit consisting of Sol(T1/ST2)ₓ . In yet another particular aspect, the two subunits of the heterodimer are operably linked to an immunoglobulin. The term “operably linked” is as defined here above. In these embodiment, the subunits are operably linked to all or a portion of an immunoglobulin, particularly a human immunoglobulin, even more particularly the Fc portion of a human immunoglobulin. Typically an Fc portion of a human immunoglobulin contains two constant region domains (the CH2 and CH3 domains) and a hinge region but lacks the variable region (See e.g. U.S. Pat. Nos. 6,018,026 and 5,750,375). The immunoglobulin may be selected from any of the major classes of immunoglobulins, including IgA, IgD, IgE, IgG and IgM, and any subclass or isotype, e.g. IgG1, IgG2, IgG3 and IgG4; IgA-1 and IgA-2. In an embodiment, the Fc moiety is of human IgG4, which is stable in solution and has little or no complement activating activity. In another embodiment, the Fc moiety is of human IgG1. The Fc part may be mutated in order to prevent unwanted activities, such as complement binding, binding to Fc receptors, or the like. Usually the two subunits are operably linked to the same immunoglobulin (particularly to the Fc portion of a human immunoglobulin, for example of a human IgG4 or human IgG1). The amino acid sequence derived from the immunoglobulin may be linked to the C-terminus or to the N-terminus of the subunit, preferably to the C-terminus. Such fusion proteins can be prepared by transfecting cells with DNA encoding the first subunit:Fc fusion protein and DNA encoding the other subunit:Fc fusion protein and expressing the dimers in the same cells. Subunits advantageously comprise a native or heterologous signal peptide when initially synthesized, to promote secretion from the cell, but the signal sequence is cleaved upon secretion. Methods for making immunoglobulin fusion proteins are well known in the art, such as the ones described in Hollenbaugh and Aruffo (“Construction of Immunoglobulin Fusion Proteins”, in Current Protocols in Immunology, Suppl. 4, pages 10.19.1-10.19.11, 1992) or WO 01/03737, for example.

Alternatively, the heterodimers comprising one subunit consisting of Sol(IL-18Rα) and one subunit consisting of Sol(T1/ST2), or one subunit consisting of Sol(IL-18Rα)ₓ and one subunit consisting of Sol(T1/ST2), or one subunit consisting of Sol(IL-18Rα) and one subunit consisting of Sol(T1/ST2)ₓ, or one subunit consisting of Sol(IL-18Rα)ₓ and one subunit consisting of Sol(T1/ST2)ₓ, of the present invention can be prepared by operably linking one of the receptor subunit to the constant region of an immunoglobulin heavy chain and operably linking the other receptor subunit to the constant region of an immunoglobulin light chain. The term “operably linked” is as defined here above. For example, the Sol(IL-18Rα) or Sol(IL-18Rα)ₓ subunit can be operably linked to the CH₁-hinge-CH2-CH3 region of human IgG1 and the Sol(T1/ST2) subunit can be operably linked to the C kappa region of the Ig kappa light chain (or vice versa); or the Sol(IL-18Rα) or Sol(IL-18Rα)ₓ subunit can be operably linked to the CH₁-hinge-CH2-CH3 region of human IgG1 and the Sol(T1/ST2)ₓ subunit can be operably linked to the C kappa region of the Ig kappa light chain (or vice versa). The amino acid sequence derived from the immunoglobulin may be linked to the C-terminus or to the N-terminus of the subunits, preferably to the C-terminus. Cells transfected with DNA encoding the immunoglobulin light chain fusion protein and the immunoglobulin heavy chain fusion protein express heavy chain/light chain heterodimers containing each a subunit. Both subunits advantageously comprise a native or heterologous signal peptide when initially synthesized, to promote secretion from the cell, but the signal sequence is cleaved upon secretion.

In another particular aspect of the present invention, the subunits of the heteromultimers are linked via non-covalent linkages. Non-covalent bonding of the subunits may be achieved by any suitable means that does not interfere with its biological activity (i.e. its ability to reduce the symptoms of MS). In a particular aspect, these heteromultimers are heterodimers comprising one subunit consisting of Sol(IL-18Rα) and one subunit consisting of Sol(T1/ST2), or one subunit consisting of Sol(IL-18Rα)ₓ and one subunit consisting of Sol(T1/ST2), or one subunit consisting of Sol(IL-18Rα) and one subunit consisting of Sol(T1/ST2)ₓ, or one subunit consisting of Sol(IL-18Rα)ₓ and one subunit consisting of Sol(T1/ST2)ₓ, where one subunit is operably linked to a first compound the other is operably linked to a second compound that will non-covalently bond to the first compound. The term “operably linked” is as defined here above. Examples of such compounds are biotin and avidin. These heterodimers can be prepared by operably linking one of the receptor subunit to biotin and operably linking the other subunit to avidin. The receptor is thus formed through the non-covalent interactions of biotin with avidin. Other examples include subunits of heterodimeric proteinaceous hormone. In these embodiments, a DNA construct encoding one subunit (Sol(IL-18Rα) or Sol(IL-18Rα)ₓ) is fused to a DNA construct encoding a subunit of a heterodimeric proteinaceous hormone, such as hCG, and a DNA construct encoding the other subunit (Sol(T1/ST2) or Sol(T1/ST2)ₓ) is fused to DNA encoding the other subunit of the heterodimeric proteinaceous hormone, such as hCG (as disclosed in U.S. Pat. No. 6,193,972). These DNA constructs are coexpressed in the same cells leading to the expression of an heterodimeric receptor fusion protein, as each coexpressed sequence contains a corresponding hormone subunit so as to form a heterodimer upon expression. The amino acid sequence derived from the heterodimeric proteinaceous hormone may be linked to the C-terminus or to the N-terminus of the subunits, preferably to the C-terminus. Both subunits advantageously comprise a native or heterologous signal peptide when initially synthesized, to promote secretion from the cell, but the signal sequence is cleaved upon secretion.

Other examples for protein sequences allowing the dimerization of the Sol(IL-18Rα)ₓ-(T1/ST2)ₓ subunits are domains isolated from proteins such as collagen X (WO 04/33486), C4BP (WO 04/20639), Erb proteins (WO 98/02540), or coiled coil peptides (WO 01/00814).

In an embodiment, the heteromultimers comprising at least one Sol(IL-18Rα) or Sol(IL-18Rα)ₓ subunit and one Sol(T1/ST2) or Sol(T1/ST2)ₓ subunit of the present invention are recombinant antibodies. The technology of recombinant antibody is described for example in the U.S. Pat. No. 6,018,026. In that case, the multimer of one Sol(IL-18Rα) or Sol(IL-18Rα)ₓ and Sol(T1/ST2) or Sol(T1/ST2)ₓ is a multimer polypeptide fusion, comprising: a first Sol(IL-18Rα) or Sol(IL-18Rα)ₓ polypeptide chain and a second Sol(T1/ST2) or Sol(T1/ST2)ₓ polypeptide chains, wherein one of the polypeptide chain is operably linked to an immunoglobulin heavy chain constant region and the other polypeptide chain is operably linked to an immunoglobulin light chain constant region. In an embodiment, the first Sol(IL-18Rα) or Sol(IL-18Rα)ₓ polypeptide chain is operably linked to an immunoglobulin heavy chain constant region and the second Sol(T1/ST2) or Sol(T1/ST2)ₓ polypeptide chains is operably linked to an immunoglobulin light chain constant region. In another embodiment, the first Sol(IL-18Rα) or Sol(IL-18Rα)ₓ polypeptide chain is operably linked to an immunoglobulin light chain constant region and the second Sol(T1/ST2) or Sol(T1/ST2)ₓ polypeptide chains is operably linked to an immunoglobulin heavy chain constant region. The term “operably linked” indicates that Sol(IL-18Rα) or Sol(IL-18Rα)ₓ and Sol(T1/ST2) or Sol(T1/ST2)ₓ, and the immunoglobulin heavy or light chain constant region are associated through peptide linkage, either directly or via a “peptide linker” (as defined here above). In an embodiment, the immunoglobulin heavy chain constant region domain and the immunoglobulin light chain constant region domain are human immunoglobulin constant regions. In an embodiment, the immunoglobulin heavy chain constant region domain is selected from the group consisting of the constant region of an α, γ, μ, δ or ε human immunoglobulin heavy chain. Preferably, said constant region is the constant region of a γ1, γ2, γ3 or γ4 human immunoglobulin heavy chain. In a preferred embodiment, the immunoglobulin light chain constant region domain is selected from the group consisting of the constant region of a κ or λ human immunoglobulin light chain. The amino acid sequence from the immunoglobulin may be linked to the C-terminus or to the N-terminus of the Sol(IL-18Rα) or Sol(IL-18Rα)ₓ and Sol(T1/ST2) or Sol(T1/ST2)ₓ subunits, preferably to the C-terminus. Cells transfected with DNA encoding the immunoglobulin light chain fusion protein and the immunoglobulin heavy chain fusion protein express a fusion protein having the structure of an antibody. The resulting protein obtained consists of:

two identical heavy chains constant region operably linked to a Sol(IL-18Rα) or Sol(IL-18Rα)ₓ subunit and two identical light chains constant region operably linked to a Sol(T1/ST2) or Sol(T1/ST2)ₓ subunit; or

two identical heavy chains constant region operably linked to a Sol(T1/ST2) or Sol(T1/ST2)ₓ subunit and two identical light chains constant region operably linked to a Sol(IL-18Rα) or Sol(IL-18Rα)ₓ subunit.

As for an antibody, heavy and light chains are disulfide linked (interchain disulfide bond) and heavy chains are disulfide linked (interchain disulfide bond).
The resulting molecule is therefore a homodimer composed of two heterodimers each of these heterodimers being composed of:

an immunoglobulin heavy chain constant region operably linked to a Sol(IL-18Rα) or Sol(IL-18Rα)ₓ polypeptide chain and;

an immunoglobulin light chain constant region operably linked to a Sol(T1/ST2) or Sol(T1/ST2)ₓ polypeptide chain.
Or a homodimer composed of two heterodimers each of these heterodimers being composed of:

an immunoglobulin heavy chain constant region operably linked to a Sol(T1/ST2) or Sol(T1/ST2)ₓ polypeptide chain and; an immunoglobulin light chain constant region operably linked to a Sol(IL-18Rα) or Sol(IL-18Rα)ₓ polypeptide chain.

Both subunits advantageously comprise a native or heterologous signal peptide when initially synthesized, to promote secretion from the cell, but the signal sequence is cleaved upon secretion. In an embodiment, the heavy constant chain is human γ4, which is stable in solution and has little or no complement activating activity. In another embodiment, the heavy constant chain is human γ1. The heavy constant chain may be mutated in order to prevent unwanted activities, such as complement binding, binding to Fc receptors, or the like.

1. In an embodiment the recombinant antibody of the present invention comprises or consists of:

two identical heavy chains constant regions, said heavy chains constant regions being the constant region of γ1, γ2, γ3 or γ4 human immunoglobulin heavy chain, operably linked to the extracellular domain of the human IL-18Rα and;

two identical light chains constant region, said light chain constant region being the constant region of κ or λ human immunoglobulin light chain, operably linked to the extra cellular domain of the human T1/ST2. In an embodiment, heavy and light chains are disulfide linked (interchain disulfide bond) and heavy chains are disulfide linked (interchain disulfide bond) as for a natural antibody.

2. In another particular embodiment, the recombinant antibody of the present invention comprises or consists of:

two identical heavy chains constant region, said heavy chains constant region being the constant region of γ1, γ2, γ3 or γ4 human immunoglobulin heavy chain, operably linked to the extracellular domain of the human T1/ST2 and;

two identical light chains constant region, said light chain constant region being the constant region of κ or λ human immunoglobulin light chain, operably linked to the extra cellular domain of the human IL-18Rα. In an embodiment, heavy and light chains are disulfide linked (interchain disulfide bond) and heavy chains are disulfide linked (interchain disulfide bond) as for a natural antibody.

3. In another embodiment, the present invention resides in a recombinant antibody as defined at point 1 or 2 above wherein the constant regions of the heavy chain are the constant regions of γ1 human immunoglobulin heavy chain.

4. In another embodiment, the present invention resides in a recombinant antibody as defined at point 1, 2 or 3 above wherein the constant regions of the light chain are the constant regions of κ human immunoglobulin light chain.

5. In another embodiment, the present invention resides in a recombinant antibody as defined at point 1, 2, 3 or 4 above wherein the extra cellular domain of the human IL-18Rα consists of amino acids residues 19-329 of SEQ ID NO: 2 or a variant of said polypeptide as defined here above.

6. In another embodiment, the present invention resides in a recombinant antibody as defined at point 1, 2, 3, 4 or 5 above wherein the extra cellular domain of the human T1/ST2 consists of amino acids residues 19-328 of SEQ ID NO: 10 or a variant of said polypeptide as defined here above.

7. In another embodiment, the present invention resides in a recombinant antibody as defined at point 1, 2, 3, 4, 5 or 6 above wherein the heavy chain constant regions are directly associated through peptide linkage to the extracellular domain of the human IL-18Rα or of the human T1/ST2.

8. In another embodiment, the present invention resides in a recombinant antibody as defined at point 1, 2, 3, 4, 5, 6 or 7 above wherein the light chain constant regions are directly associated through peptide linkage to the extracellular domain of the human IL-18Rα or of the human T1/ST2.

9. In another embodiment, the present invention resides in a recombinant antibody as defined at point 1, 2, 3, 4, 5 or 6 above wherein the heavy chain constant regions are associated through peptide linkage via a peptide linker to the extracellular domain of the human IL-18Rα or of the human T1/ST2. The peptide linker can be as short as 1 to 3 amino acid residues in length (preferably consisting of small amino acids such as glycine, serine, threonine or alanine) or longer, for example 13, 15 or 16 amino acid residues in length, introduced between the subunits. Preferably, the peptide linker is a peptide which is immunologically inert. Said linker may be a tripeptide of the sequence E-F-M (Glu-Phe-Met) (SEQ ID NO: 13), for example, a 13-amino acid linker sequence consisting of Glu-Phe-Gly-Ala-Gly-Leu-Val-Leu-Gly-Gly-Gln-Phe-Met (SEQ ID NO: 14), a 15-amino acid linker sequence consisting of (G₄S)₃ (SEQ ID NO: 15), a 16-amino acid linker sequence consisting of GGSGG SGGGG SGGGG S (SEQ ID NO: 16) or the hinge region of human IgG (e.g. IgG1, IgG2, IgG3 or IgG4). In an embodiment, said peptide linker is a 15-amino acid linker sequence consisting of (G₄S)₃ (SEQ ID NO: 15),

10. In another embodiment, the present invention resides in a recombinant antibody as defined at point 1, 2, 3, 4, 5, 6 or 9 above wherein the light chain constant regions are associated through peptide linkage via a peptide linker to the extracellular domain of the human IL-18Rα or of the human T1/ST2. The peptide linker can be as short as 1 to 3 amino acid residues in length (preferably consisting of small amino acids such as glycine, serine, threonine or alanine) or longer, for example 13, 15 or 16 amino acid residues in length, introduced between the subunits. Preferably, the peptide linker is a peptide which is immunologically inert. Said linker may be a tripeptide of the sequence E-F-M (Glu-Phe-Met) (SEQ ID NO: 13), for example, a 13-amino acid linker sequence consisting of Glu-Phe-Gly-Ala-Gly-Leu-Val-Leu-Gly-Gly-Gln-Phe-Met (SEQ ID NO: 14), a 15-amino acid linker sequence consisting of (G₄S)₃ (SEQ ID NO: 15), a 16-amino acid linker sequence consisting of GGSGG SGGGG SGGGG S (SEQ ID NO: 16) or the hinge region of human IgG (e.g. IgG1, IgG2, IgG3 or IgG4). In an embodiment, said peptide linker is a 15-amino acid linker sequence consisting of (G₄S)₃ (SEQ ID NO: 15).

11. In another embodiment, the present invention resides in a recombinant antibody as defined at point 1, 2, 3, 4, 5, 6, 7, 8, 9 or 10 above wherein the heavy constant chain is human γ4, which is stable in solution and has little or no complement activating activity.

12. In another embodiment, the present invention resides in a recombinant antibody as defined at point 1, 2, 3, 4, 5, 6, 7, 8, 9 or 10 above wherein the heavy constant chain is human γ1 and is mutated in order to prevent unwanted activities, such as complement binding, binding to Fc receptors, or the like.

13. In another embodiment, the present invention resides in a recombinant antibody as defined at point 1, 2, 3, 4, 5, 6, 7, 8, 9, 10, 11 or 12 above wherein the heavy chain constant regions are operably linked to the C-terminus or to the N-terminus of the extracellular domain of the human IL-18Rα or of the human T1/ST2, preferably to the C-terminus.

14. In another embodiment, the present invention resides in a recombinant antibody as defined at point 1, 2, 3, 4, 5, 6, 7, 8, 9, 10, 11, 12 or 13 above wherein the light chain constant regions are operably linked to the C-terminus or to the N-terminus of the extracellular domain of the human IL-18Rα or of the human T1/ST2, preferably to the C-terminus.

15. In another embodiment, the present invention resides in a recombinant antibody as defined at point 1, 2, 3, 4, 5, 6, 7, 8, 9, 10, 11, 12, 13 or 14 above wherein the extracellular domain of the human IL-18Rα or of the human T1/ST2 is operably linked to the C-terminus or to the N-terminus of the heavy chain constant regions, preferably to the N-terminus.

16. In another embodiment, the present invention resides in a recombinant antibody as defined at point 1, 2, 3, 4, 5, 6, 7, 8, 9, 10, 11, 12, 13, 14 or 15 above wherein the extracellular domain of the human IL-18Rα or of the human T1/ST2 is operably linked to the C-terminus or to the N-terminus of the light chain constant regions, preferably to the N-terminus.

Also, if needed, fusion proteins described herein may comprise any functional region facilitating purification or production. Specific examples of such additional amino acid sequences include a GST sequence or a His tag sequence.

6) Soluble IL-18Rα Comprising at Least One IL-18Rα Subunit (Sol(IL-18Rα) and/or Sol(IL-18Rα)ₓ) and One IL-1R-1 Subunit (Sol(IL-1R-1) and/or Sol(IL-1R-1)ₓ):

In a particular aspect of the present invention, the soluble IL-18Rα receptors used to treat, prevent or ameliorate the symptoms of an autoimmune or demyelinating disease, in particular MS, are soluble receptors comprising at least one IL-18Rα subunit (Sol(IL-18Rα) and/or Sol(IL-18Rα)ₓ as defined here above), and at least one IL-1R-1 subunit, as defined here after. The term “soluble receptor” has been defined above.

IL-1R-1 (also named Interleukin-1 receptor type I, IL-1RT1, IL-1R-alpha, p80 or CD121a antigen in the literature) is a member of the IL-1 receptor family and possesses an extracellular domain comprising three immunoglobulin-like domains (Ig domains). A cDNA encoding human IL-1R-1 is presented at SEQ ID NO: 17. This cDNA encodes a 569 amino acids long protein (SEQ ID NO: 18) which includes an extracellular domain of 336 amino acids (residues 1-336 from N- to C-terminus of SEQ ID NO: 18) that includes a signal peptide of 17 amino acids (residues 1-17 of SEQ ID NO: 18); a transmembrane region of 20 amino acids (residues 337-356) and a cytoplasmic domain of 213 amino acids (residues 357-569).

6.1 IL-1R-1 Subunit and Variants thereof (Named here after “Sol(IL-1R-1)”):

In one aspect, the IL-1R-1 subunit of the soluble IL-18Rα receptor of the present invention is a polypeptide comprising all or part of the extracellular domain of IL-1R-1, in particular all or part of the extracellular domain of human IL-1R-1 or a variant thereof.

In an aspect, the IL-1R-1 subunit of the soluble IL-18Rα receptor of the present invention (Sol(IL-1R-1)) is a polypeptide comprising or consisting of amino acids residues 18-336 of SEQ ID NO: 18, or a variant of said polypeptide. Ordinarily, the variant polypeptides are at least 290 amino acids in length, often at least 310 amino acids in length, more often at least 319 amino acids in length. A variant is defined as a polypeptide having at least 80% amino acid sequence identity with the sequence of reference (here residues 18-336 of SEQ ID NO: 18), preferably at least 90% amino acid sequence identity, more preferably at least 95% amino acid sequence identity, more preferably at least 98% amino acid sequence identity and most preferably at least 99% amino acid sequence identity. More preferably, the variants are differing from the sequence of reference (here residues 18-336 of SEQ ID NO: 18) by five, more preferably by four, even more preferably by three, even more preferably by two and most preferably by one amino acid. In some particular aspects of the invention, the variants are differing from the sequence of reference (here residues 18-336 of SEQ ID NO: 18) by the lack of 20, 15, 10, 9, 8, 7, 6, 5, 4, 3, 2 or 1 amino acid(s) at the N-terminal and/or C-terminal end. One of skill in the art using the genetic code can readily determine polynucleotides that encode such polypeptides. “Percent (%) amino acid sequence identity” is defined as here above.

In another embodiment, Sol(IL-1R-1) is a polypeptide comprising or consisting of amino acids residues 18-225, or 111-336, or 18-117 and 211-336 linked by a peptide bond, of SEQ ID NO: 18, or a variant of said polypeptide. Ordinarily, the variant polypeptides are at least 100 amino acids in length, often at least 126 amino acids in length, often at least 208 amino acids in length, more often at least 226 amino acids in length. A variant is defined as a polypeptide having at least 80% amino acid sequence identity with the sequence of reference (here residues 18-225, or 111-336, or 18-117 and 211-336 linked by a peptide bond, of SEQ ID NO: 18), preferably at least 90% amino acid sequence identity, more preferably at least 95% amino acid sequence identity, more preferably at least 98% amino acid sequence identity and most preferably at least 99% amino acid sequence identity. More preferably, the variants are differing from the sequence of reference (residues 18-225, or 111-336, or 18-117 and 211-336 linked by a peptide bond, of SEQ ID NO: 18), by five, more preferably by four, even more preferably by three, even more preferably by two and most preferably by one amino acid. In some particular aspects of the invention, the variants are differing from the sequence of reference (residues 18-225, or 111-336, or 18-117 and 211-336 linked by a peptide bond, of SEQ ID NO: 18), by the lack of 20, 15, 10, 9, 8, 7, 6, 5, 4, 3, 2 or 1 amino acid(s) at the N-terminal and/or C-terminal end. One of skill in the art using the genetic code can readily determine polynucleotides that encode such polypeptides. “Percent (%) amino acid sequence identity” is defined as here above.

In yet another embodiment, Sol(IL-1R-1) is a polypeptide comprising or consisting of amino acids residues 18-117, or 111-225, or 211-336 of SEQ ID NO: 18, or a variant of said polypeptide. Ordinarily, the variant polypeptides are at least 90 amino acids in length, often at least 100 amino acids in length, often at least 115 amino acids in length, more often at least 126 amino acids in length. A variant is defined as a polypeptide having at least 80% amino acid sequence identity with the sequence of reference (here residues 18-117, or 111-225, or 211-336 of SEQ ID NO: 18), preferably at least 90% amino acid sequence identity, more preferably at least 95% amino acid sequence identity, more preferably at least 98% amino acid sequence identity and most preferably at least 99% amino acid sequence identity. More preferably, the variants are differing from the sequence of reference (here residues 18-117, or 111-225, or 211-336 of SEQ ID NO: 18) by five, more preferably by four, even more preferably by three, even more preferably by two and most preferably by one amino acid. In some particular aspects of the invention, the variants are differing from the sequence of reference (here residues 18-117, or 111-225, or 211-336 of SEQ ID NO: 18), by the lack of 20, 15, 10, 9, 8, 7, 6, 5, 4, 3, 2 or 1 amino acid(s) at the N-terminal and/or C-terminal end. One of skill in the art using the genetic code can readily determine polynucleotides that encode such polypeptides. “Percent (%) amino acid sequence identity” is defined as here above.

6.2 Soluble IL-1R-1 Comprising at Least Two IL-1R-1 Subunits or Variant thereof on the Same Protein Backbone (Named here after “Sol(IL-1R-1)ₓ”):

As it will be described here after, the present invention, among other aspects, pertains to soluble IL-18Rα receptors comprising at least two IL-1R-1 subunits (at least two Sol(IL-1R-1)). These soluble IL-1R-1 comprising at least two IL-1R-1 subunits (i.e at least two Sol(IL-1R-1) subunits as defined here above) are on the same protein backbone as a fusion protein and are named here after “Sol(IL-1R-1)ₓ”. In a particular embodiment, the fusion protein comprises two Sol(IL-1R-1) subunits. In yet another particular embodiment, the at least two Sol(IL-1R-1) subunits are the same (i.e the fusion protein is a homomultimer of Sol(IL-1R-1)), and in a particular embodiment the fusion protein is a homodimer of Sol(IL-1R-1).

The at least two IL-1R-1 subunits are operably linked to one another. The term “operably linked” indicates that the subunits are associated through peptide linkage, either directly or via a “peptide linker”. In this manner, the fusion protein can be produced recombinantly, by direct expression in a host cell of a nucleic acid molecule encoding the same. The subunits are linked either directly or via a “peptide linker”. The peptide linker can be as short as 1 to 3 amino acid residues in length (preferably consisting of small amino acids such as glycine, serine, threonine or alanine) or longer, for example 13, 15 or 16 amino acid residues in length, introduced between the subunits. Preferably, the peptide linker is a peptide which is immunologically inert. Said linker may be a tripeptide of the sequence E-F-M (Glu-Phe-Met) (SEQ ID NO: 13), for example, a 13-amino acid linker sequence consisting of Glu-Phe-Gly-Ala-Gly-Leu-Val-Leu-Gly-Gly-Gln-Phe-Met (SEQ ID NO: 14), a 15-amino acid linker sequence consisting of (G₄S)₃ (SEQ ID NO: 15), a 16-amino acid linker sequence consisting of GGSGG SGGGG SGGGG S (SEQ ID NO: 16) or the hinge region of human IgG (e.g. IgG1, IgG2, IgG3 or IgG4).

6.3 Soluble IL-18Rα Comprising at Least One IL-18Rα Subunit (Sol(IL-18Rα) or Sol(IL-18Rα)ₓ) and at Least One IL-1R-1 Subunit (Sol(IL-1R-1) or Sol(IL-1R-1)ₓ):

As disclosed here above, the present invention, among other aspects, pertains to soluble IL-18Rα receptors comprising at least one IL-18Rα subunit ((Sol(IL-18Rα) or Sol(IL-18Rα)ₓ as defined here above), and one IL-1R-1 subunit (Sol(IL-1R-1) or Sol(IL-1R-1)ₓ as defined here above).

6.3.1 Soluble IL-18Rα Comprising at Least One IL-18Rα Subunit (Sol(IL-18Rα) or Sol(IL-18Rα)ₓ) and at Least One IL-1R-1 Subunit (Sol(IL-1R-1) or Sol(IL-1R-1)ₓ) on the Same Protein Backbone (Named here after “Sol(IL-18Rα)ₓ-(IL-1R-1)ₓ”):

In one aspect of the present invention, the Sol(IL-18Rα) or Sol(IL-18Rα)ₓ, and, the Sol(IL-1R-1) or Sol(IL-1R-1)ₓ, are on the same protein backbone as a fusion protein (these soluble receptors will be named “Sol(IL-18Rα)ₓ-(IL-1R-1)ₓ” here after). According to this embodiment, the Sol(IL-18Rα) or Sol(IL-18Rα)ₓ subunit is operably linked to the Sol(IL-1R-1) or Sol(IL-1R-1)ₓ subunit. The term “operably linked” indicates that the subunits are associated through peptide linkage, either directly or via a “peptide linker” (as defined here above). In this manner, the fusion protein can be produced recombinantly, by direct expression in a host cell of a nucleic acid molecule encoding the same. The Sol(IL-18Rα) or Sol(IL-18Rα)ₓ subunit can be located upstream (closer to the N-terminus of the protein) or downstream (closer to the C-terminus of the protein) to the Sol(IL-1R-1) or Sol(IL-1R-1)ₓ subunit. The subunits are linked either directly or via a “peptide linker”. In a particular embodiment, the fusion protein comprises one Sol(IL-18Rα) subunit and one Sol(IL-1R-1) subunit as defined herein.

6.3.2 Soluble IL-18Rα Comprising at Least One IL-18Rα Subunit (Sol(IL-18Rα) or Sol(IL-18Rα)ₓ) a and at Least One IL-1R-1 Subunit (Sol(IL-1R-1) or Sol(IL-1R-1)ₓ) on the Same Protein Backbone (Sol(IL-18Rα)ₓ-(IL-1R-1)ₓ) as Fusion Protein:

In yet another particular aspect, the fusion protein comprising, the Sol(IL-18Rα) or Sol(IL-18Rα)ₓ, and, the Sol(IL-1R-1) or Sol(IL-1R-1)ₓ, subunits (Sol(IL-18Rα)ₓ-(IL-1R-1)ₓ) is itself “operably linked” to an additional amino acid domain. The term “operably linked” indicates that the additional amino acid domain is associated through peptide linkage, either directly or via a “peptide linker” as defined here above. In this manner, this fusion protein can be produced recombinantly, by direct expression in a host cell of a nucleic acid molecule encoding the same. The additional amino acid domain may be located upstream (N-ter) or downstream (C-ter) to Sol(IL-18Rα)ₓ-(IL-IR-1)ₓ. In this embodiment, the additional amino acid domain comprises any functional region providing for instance an increased stability, targeting or bioavailability of the fusion protein; facilitating purification or production, or conferring on the molecule additional biological activity. Specific examples of such additional amino acid sequences include a GST sequence, a His tag sequence, the constant region of an immunoglobulin molecule or a heterodimeric protein hormone such as human chorionic gonadotropin (hCG) as described in U.S. Pat. No. 6,193,972. Also, if needed, the additional amino acid sequence included in the fusion proteins may be eliminated, either at the end of the production/purification process or in vivo, e.g., by means of an appropriate endo-/exopeptidase. For example, a spacer sequence included in the fusion protein may comprise a recognition site for an endopeptidase (such as a caspase) that can be used to separate by enzymatic cleavage the desired polypeptide variant from the additional amino acid domain, either in vivo or in vitro. In a particular aspect of this embodiment, Sol(IL-18Rα)ₓ-(IL-1R-1)ₓ comprises one Sol(IL-18Rα) subunit and one Sol(IL-1R-1) subunit as defined here above.

6.3.3 Multimers of Sol(IL-18Rα)ₓ-(IL-1R-1)ₓ:

In a particular aspect, Sol(IL-18Rα)ₓ-(IL-1R-1)ₓ soluble receptors are produced as multimers. Each subunit of the multimer comprising one Sol(IL-18Rα)ₓ-(IL-1R-1)ₓ. These multimers may be homodimeric, heterodimeric, or multimeric soluble receptors, with multimeric receptors generally not comprising more than 9 subunits, preferably not comprising more than 6 subunits, even more preferably not more than 3 subunits and most preferably not comprising more than 2 subunits. Preferably, these multimers soluble receptors are homodimers of Sol(IL-18Rα)ₓ-(IL-1R-1)ₓ as defined here above. In an embodiment, the subunits of the multimers are linked via covalent linkages. The subunits may be covalently linked by any suitable means, such as via a cross-linking reagent or a polypeptide linker. In another embodiment, the subunits are linked via non-covalent linkages.

In one embodiment, each Sol(IL-18Rα)ₓ-(IL-1R-1)ₓ subunit is operably linked to an additional amino acid domain that provides for the multimerization of the subunits (in particular the additional domains comprise any functional region providing for dimerization of the subunits). The term “operably linked” is as defined here above. The additional amino acid domain may be located upstream (N-ter) or downstream (C-ter) from the sequence of the Sol(IL-18Rα)ₓ-(IL-1R-1)ₓ subunit. In this manner, the fusion protein can be produced recombinantly, by direct expression in a host cell of a nucleic acid molecule encoding the same. In these embodiments, soluble IL-18Rα receptors of the invention are multimers of fusion proteins containing a Sol(IL-18Rα)ₓ-(IL-1R-1)ₓ subunit, operably linked to a multimerizing component capable of interacting with the multimerizing component present in another fusion protein to form a higher order structure, such as a dimer. This type of fusion proteins may be prepared by operably linking the Sol(IL-18Rα)ₓ-(IL-1R-1)ₓ subunit sequence to domains isolated from other proteins allowing the formation of dimers, trimers, etc. Examples for protein sequences allowing the multimerization of the IL-18Rα soluble receptors of the invention are domains isolated from proteins such as immunoglobulins, hCG (WO 97/30161), collagen X (WO 04/33486), C4BP (WO 04/20639), Erb proteins (WO 98/02540), or coiled coil peptides (WO 01/00814).

In a particular aspect, the multimers are dimers of Sol(IL-18Rα)ₓ-(IL-1R-1)ₓ where the subunits are operably linked to an immunoglobulin. The term “operably linked” is as defined here above. In this embodiment, the subunits are operably linked to all or a portion of an immunoglobulin, particularly a human immunoglobulin, even more particularly the Fc portion of a human immunoglobulin. Typically an Fc portion of a human immunoglobulin contains two constant region domains (the CH2 and CH3 domains) and a hinge region but lacks the variable region (See e.g. U.S. Pat. Nos. 6,018,026 and 5,750,375). The immunoglobulin may be selected from any of the major classes of immunoglobulins, including IgA, IgD, IgE, IgG and IgM, and any subclass or isotype, e.g. IgG1, IgG2, IgG3 and IgG4; IgA-1 and IgA-2. In an embodiment, the Fc moiety is of human IgG4, which is stable in solution and has little or no complement activating activity. In another embodiment, the Fc moiety is of human IgG1. The Fc part may be mutated in order to prevent unwanted activities, such as complement binding, binding to Fc receptors, or the like. Usually the Sol(IL-18Rα)ₓ-(IL-1R-1)ₓ subunits are operably linked to the same immunoglobulin (particularly to the Fc portion of a human immunoglobulin, for example of a human IgG4 or human IgG1). The amino acid sequence derived from the immunoglobulin may be linked to the C-terminus or to the N-terminus of Sol(IL-18Rα)ₓ-(IL-1R-1)ₓ, preferably to the C-terminus. Such fusion proteins can be prepared by transfecting cells with DNA encoding Sol(IL-18Rα)ₓ-(IL-1R-1)ₓ:Fc fusion protein and/or DNA encoding another Sol(IL-18Rα)ₓ-(IL-1R-1)ₓ:Fc fusion protein and expressing the dimers in the same cells. In a particular embodiment, the subunits Sol(IL-18Rα)ₓ-(IL-1R-1)ₓ are the same on each monomer (i.e the dimer is a homodimer of Sol(IL-18Rα)ₓ-(IL-1R-1)ₓ). Even more particularly, the subunits of Sol(IL-18Rα)ₓ-(IL-1R-1)ₓ are operably linked to the same immunoglobulin (particularly to the Fc portion of a human immunoglobulin, for example of a human IgG4 or human IgG1). Such fusion proteins can be prepared by transfecting cells with DNA encoding Sol(IL-18Rα)ₓ-(IL-1R-1)ₓ:Fc fusion protein and expressing the dimers in the same cells. Subunits advantageously comprise a native or heterologous signal peptide when initially synthesized, to promote secretion from the cell, but the signal sequence is cleaved upon secretion. Methods for making immunoglobulin fusion proteins are well known in the art, such as the ones described in Hollenbaugh and Aruffo (“Construction of Immunoglobulin Fusion Proteins”, in Current Protocols in Immunology, Suppl. 4, pages 10.19.1-10.19.11, 1992) or WO 01/03737, for example.

Alternatively, the dimers of Sol(IL-18Rα)ₓ-(IL-1R-1)ₓ of the present invention can be prepared by operably linking one of the receptor subunit to the constant region of an immunoglobulin heavy chain and operably linking the other receptor subunit to the constant region of an immunoglobulin light chain. The term “operably linked” indicates that Sol(IL-18Rα)ₓ-(IL-1R-1)ₓ, and the immunoglobulin are associated through peptide linkage, either directly or via a “peptide linker” (as defined here above). For example, a Sol(IL-18Rα)ₓ-(IL-1R-1)ₓ subunit can be operably linked to the CH₁-hinge-CH2-CH3 region of human IgG1 and another or the same Sol(IL-18Rα)ₓ-(IL-1R-1)ₓ subunit can be operably linked to the C kappa region of the Ig kappa light chain. The amino acid sequence derived from the immunoglobulin may be linked to the C-terminus or to the N-terminus of the Sol(IL-18Rα)ₓ-(IL-1R-1)ₓ subunits, preferably to the C-terminus. Cells transfected with DNA encoding the immunoglobulin light chain fusion protein and the immunoglobulin heavy chain fusion protein express heavy chain/light chain heterodimers containing each a Sol(IL-18Rα)ₓ-(IL-1R-1)ₓ subunit. Both subunits advantageously comprise a native or heterologous signal peptide when initially synthesized, to promote secretion from the cell, but the signal sequence is cleaved upon secretion. In a particular embodiment, the subunits Sol(IL-18Rα)ₓ-(IL-1R-1)ₓ are the same on each monomer (i.e the dimer is a homodimer of Sol(IL-18Rα)ₓ-(IL-1R-1)ₓ).

In another particular aspect of the present invention, the subunits of the multimers Sol(IL-18Rα)ₓ-(IL-1R-1)ₓ (as defined here above) are linked via non-covalent linkages. Non-covalent bonding of the subunits may be achieved by any suitable means that does not interfere with its biological activity (i.e. its ability to reduce the symptoms of MS). In a particular aspect, these multimers are dimers of Sol(IL-18Rα)ₓ-(IL-1R-1)ₓ where one subunit of Sol(IL-18Rα)ₓ-(IL-1R-1)ₓ is operably linked to a first compound and another or the same subunit Sol(IL-18Rα)ₓ-(IL-1R-1)ₓ is operably linked to a second compound that will non-covalently bond to the first compound. The term “operably linked” is as defined here above. Examples of such compounds are biotin and avidin. The dimers of Sol(IL-18Rα)ₓ-(IL-1R-1)ₓ can be prepared by operably linking one of the receptor subunit to biotin and operably linking the other subunit to avidin. The receptor is thus formed through the non-covalent interactions of biotin with avidin. Other examples include subunits of heterodimeric proteinaceous hormone. In these embodiments, a DNA construct encoding one subunit of Sol(IL-18Rα)ₓ-(IL-1R-1)ₓ is fused to a DNA construct encoding a subunit of a heterodimeric proteinaceous hormone, such as hCG, and a DNA construct encoding the other Sol(IL-18Rα)ₓ-(IL-1R-1)ₓ subunit is fused to DNA encoding the other subunit of the heterodimeric proteinaceous hormone, such as hCG (as disclosed in U.S. Pat. No. 6,193,972). These DNA constructs are coexpressed in the same cells leading to the expression of an Sol(IL-18Rα)ₓ-(IL-1R-1)ₓ heterodimeric receptor fusion protein, as each coexpressed sequence contains a corresponding hormone subunit so as to form a heterodimer upon expression. The amino acid sequence derived from the heterodimeric proteinaceous hormone may be linked to the C-terminus or to the N-terminus of the Sol(IL-18Rα)ₓ-(IL-1R-1)ₓ subunits, preferably to the C-terminus. Both subunits advantageously comprise a native or heterologous signal peptide when initially synthesized, to promote secretion from the cell, but the signal sequence is cleaved upon secretion. In a particular embodiment, the subunits Sol(IL-18Rα)ₓ-(IL-1R-1)ₓ are the same on each monomer (i.e the dimer is a homodimer of Sol(IL-18Rα)ₓ-(IL-1R-1)ₓ).

Other examples for protein sequences allowing the dimerization of the Sol(IL-18Rα)ₓ-(IL-1R-1)ₓ subunits are domains isolated from proteins such as collagen X (WO 04/33486), C4BP (WO 04/20639), Erb proteins (WO 98/02540), or coiled coil peptides (WO 01/00814).

Also, if needed, fusion proteins described herein may comprise any functional region facilitating purification or production. Specific examples of such additional amino acid sequences include a GST sequence or a His tag sequence.

6.3.4 Soluble IL-18Rα Comprising at Least One IL-18Rα Subunit (Sol(IL-18Rα) or Sol(IL-18Rα)ₓ) and at Least One IL-1R-1 Subunit (Sol(IL-1R-1) or Sol(IL-1R-1)ₓ) as Heteromultimers:

In a particular aspect, soluble receptors of the present invention comprising at least one IL-18Rα subunit (Sol(IL-18Rα) or Sol(IL-18Rα)ₓ) and at least one IL-1R-1 subunit (Sol(IL-1R-1) or Sol(IL-1R-1)ₓ) are heteromultimers. Each subunit of the heteromultimer comprising:

at least one IL-18Rα subunit (Sol(IL-18Rα) or Sol(IL-18Rα)ₓ) or;

at least one IL-1R-1 subunit (Sol(IL-1R-1) or Sol(IL-1R-1)ₓ).

These heteromultimers generally do not comprise more than 9 subunits, preferably not more than 6 subunits, even more preferably not more than 3 subunits and most preferably not more than 2 subunits. Preferably, these heteromultimers soluble receptors are heterodimers comprising one subunit consisting of Sol(IL-18Rα) or Sol(IL-18Rα)ₓ (as defined above) and one subunit consisting of Sol(IL-1R-1) or Sol(IL-1R-1)ₓ (as defined above). In an embodiment, the subunits of the heteromultimers are linked via covalent linkages. The subunits may be covalently linked by any suitable means, such as via a cross-linking reagent. In another embodiment, the subunits are linked via non-covalent linkages.

In one embodiment, each subunit of the heteromultimer is operably linked to an additional amino acid domain that provides for the multimerization of the subunits (in particular the additional domains may comprise any functional region providing for dimerization of the subunits). The term “operably linked” is as defined here above. The additional amino acid domain may be located upstream (N-ter) or downstream (C-ter) (preferably downstream (C-ter)) from the sequence of the Sol(IL-18Rα) or Sol(IL-18Rα)ₓ subunit(s) and upstream (N-ter) or downstream (C-ter) (preferably downstream (C-ter)) from the sequence of the Sol(IL-1R-1) or Sol(IL-1R-1)ₓ subunit(s). In this manner, the fusion protein can be produced recombinantly, by direct expression in a host cell of a nucleic acid molecule encoding the same. In these embodiments, soluble IL-18Rα receptors of the invention are heteromultimers of fusion proteins containing one subunit consisting of Sol(IL-18Rα) or Sol(IL-18Rα)ₓ or of Sol(IL-1R-1) or Sol(IL-IR-1)ₓ, operably linked to a multimerizing component capable of interacting with the multimerizing component present in another fusion protein to form a higher order structure, such as a dimer. This type of fusion proteins may be prepared by operably linking the Sol(IL-18Rα) or Sol(IL-18Rα)ₓ subunit sequence and the Sol(IL-1R-1) or Sol(IL-1R-1)ₓ subunit sequence to domains isolated from other proteins allowing the formation of dimers, trimers, etc. Examples for protein sequences allowing the multimerization of the IL-18Rα soluble receptors of the invention are domains isolated from proteins such as immunoglobulins, hCG (WO 97/30161), collagen X (WO 04/33486), C4BP (WO 04/20639), Erb proteins (WO 98/02540), or coiled coil peptides (WO 01/00814).

In a particular aspect, the heteromultimers are heterodimers comprising one subunit consisting of Sol(IL-18Rα) and one subunit consisting of Sol(IL-1R-1), or one subunit consisting of Sol(IL-18Rα)ₓ and one subunit consisting of Sol(IL-1R-1), or one subunit consisting of Sol(IL-18Rα) and one subunit consisting of Sol(IL-1R-1)ₓ, or one subunit consisting of Sol(IL-18Rα)ₓ and one subunit consisting of Sol(IL-1R-1)ₓ. In yet another particular aspect, the two subunits of the heterodimer are operably linked to an immunoglobulin. The term “operably linked” is as defined here above. In these embodiment, the subunits are operably linked to all or a portion of an immunoglobulin, particularly a human immunoglobulin, even more particularly the Fc portion of a human immunoglobulin. Typically an Fc portion of a human immunoglobulin contains two constant region domains (the CH2 and CH3 domains) and a hinge region but lacks the variable region (See e.g. U.S. Pat. Nos. 6,018,026 and 5,750,375). The immunoglobulin may be selected from any of the major classes of immunoglobulins, including IgA, IgD, IgE, IgG and IgM, and any subclass or isotype, e.g. IgG1, IgG2, IgG3 and IgG4; IgA-1 and IgA-2. In an embodiment, the Fc moiety is of human IgG4, which is stable in solution and has little or no complement activating activity. In another embodiment, the Fc moiety is of human IgG1. The Fc part may be mutated in order to prevent unwanted activities, such as complement binding, binding to Fc receptors, or the like. Usually the two subunits are operably linked to the same immunoglobulin (particularly to the Fc portion of a human immunoglobulin, for example of a human IgG4 or human IgG1). The amino acid sequence derived from the immunoglobulin may be linked to the C-terminus or to the N-terminus of the subunit, preferably to the C-terminus. Such fusion proteins can be prepared by transfecting cells with DNA encoding the first subunit:Fc fusion protein and DNA encoding the other subunit:Fc fusion protein and expressing the dimers in the same cells. Subunits advantageously comprise a native or heterologous signal peptide when initially synthesized, to promote secretion from the cell, but the signal sequence is cleaved upon secretion. Methods for making immunoglobulin fusion proteins are well known in the art, such as the ones described in Hollenbaugh and Aruffo (“Construction of Immunoglobulin Fusion Proteins”, in Current Protocols in Immunology, Suppl. 4, pages 10.19.1-10.19.11, 1992) or WO 01/03737, for example.

Alternatively, the heterodimers comprising one subunit consisting of Sol(IL-18Rα) and one subunit consisting of Sol(IL-1R-1), or one subunit consisting of Sol(IL-18Rα)ₓ and one subunit consisting of Sol(IL-1R-1), or one subunit consisting of Sol(IL-18Rα) and one subunit consisting of Sol(IL-1R-1)ₓ, or one subunit consisting of Sol(IL-18Rα)ₓ and one subunit consisting of Sol(IL-1R-1)ₓ, of the present invention can be prepared by operably linking one of the receptor subunit to the constant region of an immunoglobulin heavy chain and operably linking the other receptor subunit to the constant region of an immunoglobulin light chain. The term “operably linked” is as defined here above. For example, the Sol(IL-18Rα) or Sol(IL-18Rα)ₓ subunit can be operably linked to the CH₁-hinge-CH2-CH3 region of human IgG1 and the Sol(IL-1R-1) subunit can be operably linked to the C kappa region of the Ig kappa light chain (or vice versa); or the Sol(IL-18Rα) or Sol(IL-18Rα)ₓ subunit can be operably linked to the CH₁-hinge-CH2-CH3 region of human IgG1 and the Sol(IL-1R-1)ₓ subunit can be operably linked to the C kappa region of the Ig kappa light chain (or vice versa). The amino acid sequence derived from the immunoglobulin may be linked to the C-terminus or to the N-terminus of the subunits, preferably to the C-terminus. Cells transfected with DNA encoding the immunoglobulin light chain fusion protein and the immunoglobulin heavy chain fusion protein express heavy chain/light chain heterodimers containing each a subunit. Both subunits advantageously comprise a native or heterologous signal peptide when initially synthesized, to promote secretion from the cell, but the signal sequence is cleaved upon secretion.

In another particular aspect of the present invention, the subunits of the heteromultimers are linked via non-covalent linkages. Non-covalent bonding of the subunits may be achieved by any suitable means that does not interfere with its biological activity (i.e. its ability to reduce the symptoms of MS). In a particular aspect, these heteromultimers are heterodimers comprising one subunit consisting of Sol(IL-18Rα) and one subunit consisting of Sol(IL-1R-1), or one subunit consisting of Sol(IL-18Rα)ₓ and one subunit consisting of Sol(IL-1R-1), or one subunit consisting of Sol(IL-18Rα) and one subunit consisting of Sol(IL-1R-1)ₓ, or one subunit consisting of Sol(IL-18Rα)ₓ and one subunit consisting of Sol(IL-1R-1)ₓ, where one subunit is operably linked to a first compound the other is operably linked to a second compound that will non-covalently bond to the first compound. The term “operably linked” is as defined here above. Examples of such compounds are biotin and avidin. These heterodimers can be prepared by operably linking one of the receptor subunit to biotin and operably linking the other subunit to avidin. The receptor is thus formed through the non-covalent interactions of biotin with avidin. Other examples include subunits of heterodimeric proteinaceous hormone. In these embodiments, a DNA construct encoding one subunit (Sol(IL-18Rα) or Sol(IL-18Rα)ₓ) is fused to a DNA construct encoding a subunit of a heterodimeric proteinaceous hormone, such as hCG, and a DNA construct encoding the other subunit (Sol(IL-1R-1) or Sol(IL-1R-1)ₓ) is fused to DNA encoding the other subunit of the heterodimeric proteinaceous hormone, such as hCG (as disclosed in U.S. Pat. No. 6,193,972). These DNA constructs are coexpressed in the same cells leading to the expression of an heterodimeric receptor fusion protein, as each coexpressed sequence contains a corresponding hormone subunit so as to form a heterodimer upon expression. The amino acid sequence derived from the heterodimeric proteinaceous hormone may be linked to the C-terminus or to the N-terminus of the subunits, preferably to the C-terminus. Both subunits advantageously comprise a native or heterologous signal peptide when initially synthesized, to promote secretion from the cell, but the signal sequence is cleaved upon secretion.

Other examples for protein sequences allowing the dimerization of the Sol(IL-18Rα)ₓ-(IL-1R-1)ₓ subunits are domains isolated from proteins such as collagen X (WO 04/33486), C4BP (WO 04/20639), Erb proteins (WO 98/02540), or coiled coil peptides (WO 01/00814).

In an embodiment, the heteromultimers comprising at least one Sol(IL-18Rα) or Sol(IL-18Rα)ₓ subunit and one Sol(IL-1R-1) or Sol(IL-1R-1)ₓ subunit of the present invention are recombinant antibodies. The technology of recombinant antibody is described for example in the U.S. Pat. No. 6,018,026. In that case, the multimer of one Sol(IL-18Rα) or Sol(IL-18Rα)ₓ and Sol(IL-1R-1) or Sol(IL-1R-1)ₓ is a multimer polypeptide fusion, comprising: a first Sol(IL-18Rα) or Sol(IL-18Rα)ₓ polypeptide chain and a second Sol(IL-1R-1) or Sol(IL-1R-1)ₓ polypeptide chains, wherein one of the polypeptide chain is operably linked to an immunoglobulin heavy chain constant region and the other polypeptide chain is operably linked to an immunoglobulin light chain constant region. In an embodiment, the first Sol(IL-18Rα) or Sol(IL-18Rα)ₓ polypeptide chain is operably linked to an immunoglobulin heavy chain constant region and the second Sol(IL-1R-1) or Sol(IL-1R-1)ₓ polypeptide chains is operably linked to an immunoglobulin light chain constant region. In another embodiment, the first Sol(IL-18Rα) or Sol(IL-18Rα)ₓ polypeptide chain is operably linked to an immunoglobulin light chain constant region and the second Sol(IL-1R-1) or Sol(IL-1R-1)ₓ polypeptide chains is operably linked to an immunoglobulin heavy chain constant region. The term “operably linked” indicates that Sol(IL-18Rα) or Sol(IL-18Rα)ₓ and Sol(IL-1R-1) or Sol(IL-1R-1)ₓ, and the immunoglobulin heavy or light chain constant region are associated through peptide linkage, either directly or via a “peptide linker” (as defined here above). In an embodiment, the immunoglobulin heavy chain constant region domain and the immunoglobulin light chain constant region domain are human immunoglobulin constant regions. In an embodiment, the immunoglobulin heavy chain constant region domain is selected from the group consisting of the constant region of an α, γ, μ, δ or ε human immunoglobulin heavy chain. Preferably, said constant region is the constant region of a γ1, γ2, γ3 or γ4 human immunoglobulin heavy chain. In a preferred embodiment, the immunoglobulin light chain constant region domain is selected from the group consisting of the constant region of a κ or λ human immunoglobulin light chain. The amino acid sequence from the immunoglobulin may be linked to the C-terminus or to the N-terminus of the Sol(IL-18Rα) or Sol(IL-18Rα)ₓ and Sol(IL-1R-1) or Sol(IL-1R-1)ₓ subunits, preferably to the C-terminus. Cells transfected with DNA encoding the immunoglobulin light chain fusion protein and the immunoglobulin heavy chain fusion protein express a fusion protein having the structure of an antibody. The resulting protein obtained consists of:

two identical heavy chains constant region operably linked to a Sol(IL-18Rα) or Sol(IL-18Rα)ₓ subunit and two identical light chains constant region operably linked to a Sol(IL-1R-1) or Sol(IL-1R-1)ₓ subunit; or

two identical heavy chains constant region operably linked to a Sol(IL-1R-1) or Sol(IL-IR-1)ₓ subunit and two identical light chains constant region operably linked to a Sol(IL-18Rα) or Sol(IL-18Rα)ₓ subunit.

As for an antibody, heavy and light chains are disulfide linked (interchain disulfide bond) and heavy chains are disulfide linked (interchain disulfide bond).
The resulting molecule is therefore a homodimer composed of two heterodimers each of these heterodimers being composed of:

an immunoglobulin heavy chain constant region operably linked to a Sol(IL-18Rα) or Sol(IL-18Rα)ₓ polypeptide chain and;

an immunoglobulin light chain constant region operably linked to a Sol(IL-1R-1) or Sol(IL-1R-1)ₓ polypeptide chain.
Or a homodimer composed of two heterodimers each of these heterodimers being composed of:

an immunoglobulin heavy chain constant region operably linked to a Sol(IL-1R-1) or Sol(IL-1R-1)ₓ polypeptide chain and;

an immunoglobulin light chain constant region operably linked to a Sol(IL-18Rα) or Sol(IL-18Rα)ₓ polypeptide chain.
Both subunits advantageously comprise a native or heterologous signal peptide when initially synthesized, to promote secretion from the cell, but the signal sequence is cleaved upon secretion. In an embodiment, the heavy constant chain is human γ4, which is stable in solution and has little or no complement activating activity. In another embodiment, the heavy constant chain is human γ1. The heavy constant chain may be mutated in order to prevent unwanted activities, such as complement binding, binding to Fc receptors, or the like.

1. In an embodiment the recombinant antibody of the present invention comprises or consists of:

two identical heavy chains constant regions, said heavy chains constant regions being the constant region of γ1, γ2, γ3 or γ4 human immunoglobulin heavy chain, operably linked to the extracellular domain of the human IL-18Rα and;

two identical light chains constant region, said light chain constant region being the constant region of κ or λ human immunoglobulin light chain, operably linked to the extra cellular domain of the human IL-1R-1. In an embodiment, heavy and light chains are disulfide linked (interchain disulfide bond) and heavy chains are disulfide linked (interchain disulfide bond) as for a natural antibody.

2. In another particular embodiment, the recombinant antibody of the present invention comprises or consists of:

two identical heavy chains constant region, said heavy chains constant region being the constant region of γ1, γ2, γ3 or γ4 human immunoglobulin heavy chain, operably linked to the extracellular domain of the human IL-1R-1 and;

two identical light chains constant region, said light chain constant region being the constant region of κ or λ human immunoglobulin light chain, operably linked to the extra cellular domain of the human IL-18Rα. In an embodiment, heavy and light chains are disulfide linked (interchain disulfide bond) and heavy chains are disulfide linked (interchain disulfide bond) as for a natural antibody.

3. In another embodiment, the present invention resides in a recombinant antibody as defined at point 1 or 2 above wherein the constant regions of the heavy chain are the constant regions of γ1 human immunoglobulin heavy chain.

4. In another embodiment, the present invention resides in a recombinant antibody as defined at point 1, 2 or 3 above wherein the constant regions of the light chain are the constant regions of κ human immunoglobulin light chain.

5. In another embodiment, the present invention resides in a recombinant antibody as defined at point 1, 2, 3 or 4 above wherein the extra cellular domain of the human IL-18Rα consists of amino acids residues 19-329 of SEQ ID NO: 2 or a variant of said polypeptide as defined here above.

6. In another embodiment, the present invention resides in a recombinant antibody as defined at point 1, 2, 3, 4 or 5 above wherein the extra cellular domain of the human IL-1R-1 consists of amino acids residues 18-336 of SEQ ID NO: 18 or a variant of said polypeptide as defined here above.

7. In another embodiment, the present invention resides in a recombinant antibody as defined at point 1, 2, 3, 4, 5 or 6 above wherein the heavy chain constant regions are directly associated through peptide linkage to the extracellular domain of the human IL-18Rα or of the human IL-1R-1.

8. In another embodiment, the present invention resides in a recombinant antibody as defined at point 1, 2, 3, 4, 5, 6 or 7 above wherein the light chain constant regions are directly associated through peptide linkage to the extracellular domain of the human IL-18Rα or of the human IL-1R-1.

9. In another embodiment, the present invention resides in a recombinant antibody as defined at point 1, 2, 3, 4, 5 or 6 above wherein the heavy chain constant regions are associated through peptide linkage via a peptide linker to the extracellular domain of the human IL-18Rα or of the human IL-1R-1. The peptide linker can be as short as 1 to 3 amino acid residues in length (preferably consisting of small amino acids such as glycine, serine, threonine or alanine) or longer, for example 13, 15 or 16 amino acid residues in length, introduced between the subunits. Preferably, the peptide linker is a peptide which is immunologically inert. Said linker may be a tripeptide of the sequence E-F-M (Glu-Phe-Met) (SEQ ID NO: 13), for example, a 13-amino acid linker sequence consisting of Glu-Phe-Gly-Ala-Gly-Leu-Val-Leu-Gly-Gly-Gln-Phe-Met (SEQ ID NO: 14), a 15-amino acid linker sequence consisting of (G₄S)₃ (SEQ ID NO: 15), a 16-amino acid linker sequence consisting of GGSGG SGGGG SGGGG S (SEQ ID NO: 16) or the hinge region of human IgG (e.g. IgG1, IgG2, IgG3 or IgG4). In an embodiment, said peptide linker is a 15-amino acid linker sequence consisting of (G₄S)₃ (SEQ ID NO: 15),

10. In another embodiment, the present invention resides in a recombinant antibody as defined at point 1, 2, 3, 4, 5, 6 or 9 above wherein the light chain constant regions are associated through peptide linkage via a peptide linker to the extracellular domain of the human IL-18Rα or of the human IL-1R-1. The peptide linker can be as short as 1 to 3 amino acid residues in length (preferably consisting of small amino acids such as glycine, serine, threonine or alanine) or longer, for example 13, 15 or 16 amino acid residues in length, introduced between the subunits. Preferably, the peptide linker is a peptide which is immunologically inert. Said linker may be a tripeptide of the sequence E-F-M (Glu-Phe-Met) (SEQ ID NO: 13), for example, a 13-amino acid linker sequence consisting of Glu-Phe-Gly-Ala-Gly-Leu-Val-Leu-Gly-Gly-Gln-Phe-Met (SEQ ID NO: 14), a 15-amino acid linker sequence consisting of (G₄S)₃ (SEQ ID NO: 15), a 16-amino acid linker sequence consisting of GGSGG SGGGG SGGGG S (SEQ ID NO: 16) or the hinge region of human IgG (e.g. IgG1, IgG2, IgG3 or IgG4). In an embodiment, said peptide linker is a 15-amino acid linker sequence consisting of (G₄S)₃ (SEQ ID NO: 15).

11. In another embodiment, the present invention resides in a recombinant antibody as defined at point 1, 2, 3, 4, 5, 6, 7, 8, 9 or 10 above wherein the heavy constant chain is human γ4, which is stable in solution and has little or no complement activating activity.

12. In another embodiment, the present invention resides in a recombinant antibody as defined at point 1, 2, 3, 4, 5, 6, 7, 8, 9 or 10 above wherein the heavy constant chain is human γ1 and is mutated in order to prevent unwanted activities, such as complement binding, binding to Fc receptors, or the like.

13. In another embodiment, the present invention resides in a recombinant antibody as defined at point 1, 2, 3, 4, 5, 6, 7, 8, 9, 10, II or 12 above wherein the heavy chain constant regions are operably linked to the C-terminus or to the N-terminus of the extracellular domain of the human IL-18Rα or of the human IL-1R-1, preferably to the C-terminus.

14. In another embodiment, the present invention resides in a recombinant antibody as defined at point 1, 2, 3, 4, 5, 6, 7, 8, 9, 10, 11, 12 or 13 above wherein the light chain constant regions are operably linked to the C-terminus or to the N-terminus of the extracellular domain of the human IL-18Rα or of the human IL-1R-1, preferably to the C-terminus.

15. In another embodiment, the present invention resides in a recombinant antibody as defined at point 1, 2, 3, 4, 5, 6, 7, 8, 9, 10, 11, 12, 13 or 14 above wherein the extracellular domain of the human IL-18Rα or of the human IL-1R-1 is operably linked to the C-terminus or to the N-terminus of the heavy chain constant regions, preferably to the N-terminus.

16. In another embodiment, the present invention resides in a recombinant antibody as defined at point 1, 2, 3, 4, 5, 6, 7, 8, 9, 10, 11, 12, 13, 14 or 15 above wherein the extracellular domain of the human IL-18Rα or of the human IL-1R-1 is operably linked to the C-terminus or to the N-terminus of the light chain constant regions, preferably to the N-terminus.

Also, if needed, fusion proteins described herein may comprise any functional region facilitating purification or production. Specific examples of such additional amino acid sequences include a GST sequence or a His tag sequence.

### 7) Preparation of the Soluble Receptors of the Present Invention:

Soluble IL-18Rα receptors disclosed herein may be produced by any technique known per se in the art, such as by recombinant technologies, chemical synthesis, cloning, ligations, or combinations thereof. In a particular embodiment, the soluble receptors of the present invention are produced by recombinant technologies, e.g., by expression of a corresponding nucleic acid in a suitable host cell. The polypeptide produced may be glycosylated or not, or may contain other post-translational modifications depending on the host cell type used. Many books and reviews provide teachings on how to clone and produce recombinant proteins using vectors and prokaryotic or eukaryotic host cells, such as some titles in the series “A Practical Approach” published by Oxford University Press (“DNA Cloning 2: Expression Systems”, 1995; “DNA Cloning 4: Mammalian Systems”, 1996; “Protein Expression”, 1999; “Protein Purification Techniques”, 2001).

A further object of the present invention is therefore an isolated nucleic acid molecule encoding any of the soluble receptor here above or below described, or a complementary strand or degenerate sequence thereof. In this regard, the term “nucleic acid molecule” encompasses all different types of nucleic acids, including without limitation deoxyribonucleic acids (e.g., DNA, cDNA, gDNA, synthetic DNA, etc.), ribonucleic acids (e.g., RNA, mRNA, etc.) and peptide nucleic acids (PNA). In a preferred embodiment, the nucleic acid molecule is a DNA molecule, such as a double-stranded DNA molecule or a cDNA molecule. The term “isolated” means nucleic acid molecules that have been identified and separated from at least one contaminant nucleic acid molecule with which it is ordinarily associated in the natural source. An isolated nucleic acid molecule is other than in the form or setting in which it is found in nature. Isolated nucleic acid molecules therefore are distinguished from the specific nucleic acid molecule as it exists in natural cells. A degenerate sequence designates any nucleotide sequence encoding the same amino acid sequence as a reference nucleotide sequence, but comprising a distinct nucleotide sequence as a result of the genetic code degeneracy.

A further object of this invention is a vector comprising DNA encoding any of the above or below described soluble receptors. The vector may be any cloning or expression vector, integrative or autonomously replicating, functional in any prokaryotic or eukaryotic cell. In particular, the vector may be a plasmid, cosmid, virus, phage, episome, artificial chromosome, and the like. The vector may comprise regulatory elements, such as a promoter, terminator, enhancer, selection marker, origin of replication, etc. Specific examples of such vectors include prokaryotic plasmids, such as pBR, pUC or pcDNA plasmids; viral vectors, including retroviral, adenoviral or AAV vectors; bacteriophages; baculoviruses; BAC or YAC, etc., as will be discussed below. The appropriate nucleic acid sequence may be inserted into the vector by a variety of procedures. In general, DNA is inserted into an appropriate restriction endonuclease site(s) using techniques known in the art. Construction of suitable vectors containing one or more of these components employs standard ligation techniques which are known to the skilled artisan.

A further aspect of the present invention is a recombinant host cell, wherein said cell comprises a nucleic acid molecule or a vector as defined above. The host cell may be a prokaryotic or eukaryotic cell. Examples of prokaryotic cells include bacteria, such as E. coli. Examples of eucaryotic cells are yeast cells, plant cells, mammalian cells and insect cells including any primary cell culture or established cell line (e.g., 3T3, Véro, HEK293, TN5, etc.). Suitable host cells for the expression of glycosylated proteins are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/−DHFR (CHO, Urlaub and Chasin, Proc. Natl, Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). Particularly preferred mammalian cells of the present invention are CHO cells.

As disclosed here above, the soluble receptors of the present invention may be produced by any technique known per se in the art, such as by recombinant technologies, chemical synthesis, cloning, ligations, or combinations thereof. In a particular embodiment, the soluble receptors are produced by recombinant technologies, e.g., by expression of a corresponding nucleic acid in a suitable host cell. Another object of this invention is therefore a method of producing a soluble receptor of the present invention, the method comprising culturing a recombinant host cell of the invention under conditions allowing expression of the nucleic acid molecule, and recovering the polypeptide produced. The polypeptide produced may be glycosylated or not, or may contain other post-translational modifications depending on the host cell type used. Many books and reviews provide teachings on how to clone and produce recombinant proteins using vectors and prokaryotic or eukaryotic host cells, such as some titles in the series “A Practical Approach” published by Oxford University Press (“DNA Cloning 2: Expression Systems”, 1995; “DNA Cloning 4: Mammalian Systems”, 1996; “Protein Expression”, 1999; “Protein Purification Techniques”, 2001).

The vectors to be used in the method of producing a soluble receptor according to the present invention can be episomal or non-/homologously integrating vectors, which can be introduced into the appropriate host cells by any suitable means (transformation, transfection, conjugation, protoplast fusion, electroporation, calcium phosphate-precipitation, direct microinjection, etc.). Factors of importance in selecting a particular plasmid, viral or retroviral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species. The vectors should allow the expression of the polypeptide or fusion proteins of the invention in prokaryotic or eukaryotic host cells, under the control of appropriate transcriptional initiation/termination regulatory sequences, which are chosen to be constitutively active or inducible in said cell. A cell line substantially enriched in such cells can be then isolated to provide a stable cell line.

Host cells are transfected or transformed with expression or cloning vectors described herein for protein production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

For eukaryotic host cells (e.g. yeasts, insect or mammalian cells), different transcriptional and translational regulatory sequences may be employed, depending on the nature of the host. They may be derived form viral sources, such as adenovirus, papilloma virus, Simian virus or the like, where the regulatory signals are associated with a particular gene which has a high level of expression. Examples are the TK promoter of the Herpes virus, the SV40 early promoter, the yeast gal4 gene promoter, etc. Transcriptional initiation regulatory signals may be selected which allow for repression and activation, so that expression of the genes can be modulated. The cells which have been stably transformed by the introduced DNA can be selected by also introducing one or more markers which allow for selection of host cells which contain the expression vector. The marker may also provide for phototrophy to an auxotrophic host, biocide resistance, e.g. antibiotics, or heavy metals such as copper, or the like. The selectable marker gene can be either directly linked to the DNA sequences to be expressed (e.g., on the same vector), or introduced into the same cell by co-transfection. Additional elements may also be needed for optimal synthesis of proteins of the invention.

Particularly suitable prokaryotic cells include bacteria (such as Bacillus subtilis or E. coli) transformed with a recombinant bacteriophage, plasmid or cosmid DNA expression vector. Such cells typically produce proteins comprising a N-terminal Methionine residue. Preferred cells to be used in the present invention are eukaryotic host cells, e.g. mammalian cells, such as human, monkey, mouse, and Chinese Hamster Ovary (CHO) cells, because they provide post-translational modifications to protein molecules, including correct folding or glycosylation at correct sites. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K¹; ATCC CCL61; CHO DG44 (Chasin et al., Som. Cell. Molec. Genet. 12:555, 1986)), rat pituitary cells (GH1; ATCC CCL82), HeLa S3 cells (ATCC CCL2.2), rat hepatoma cells (H-4-II-E; ATCC CRL 1548), SV40-transformed monkey kidney cells (COS-1; ATCC CRL 1650), Bowes melanoma and human hepatocellular carcinoma (for example Hep G2), murine embryonic cells (NIH-3T3; ATCC CRL 1658) and a number of other cell lines. Alternative eukaryotic host cells are yeast cells (e.g., Saccharomyces, Kluyveromyces, etc.) transformed with yeast expression vectors. Also yeast cells can carry out post-translational peptide modifications including glycosylation. A number of recombinant DNA strategies exist which utilize strong promoter sequences and high copy number of plasmids that can be utilized for production of the desired proteins in yeast. Yeast cells recognize leader sequences in cloned mammalian gene products and secrete polypeptides bearing leader sequences (i.e., pre-peptides).

For long-term, high-yield production of a recombinant polypeptide, stable expression is preferred. For example, cell lines which stably express the polypeptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type. A cell line substantially enriched in such cells can be then isolated to provide a stable cell line.

A particularly preferred method of high-yield production of a recombinant polypeptide of the present invention is through the use of dihydrofolate reductase (DHFR) amplification in DHFR-deficient CHO cells, by the use of successively increasing levels of methotrexate as described in U.S. Pat. No. 4,889,803. The polypeptide obtained may be in a glycosylated form.

Soluble receptors disclosed herein can also be expressed in other eukaryotic cells, such as avian, fungal, insect, yeast, or plant cells. The baculovirus system provides an efficient means to introduce cloned genes into insect cells. The materials for baculovirus/insect cell expression systems are commercially available in kit form from, inter alia, Invitrogen.

In addition to recombinant DNA technologies, the soluble receptors of this invention may be prepared by chemical synthesis technologies. Examples of chemical synthesis technologies are solid phase synthesis and liquid phase synthesis. As a solid phase synthesis, for example, the amino acid corresponding to the carboxy-terminus of the polypeptide to be synthesised is bound to a support which is insoluble in organic solvents and, by alternate repetition of reactions (e.g., by sequential condensation of amino acids with their amino groups and side chain functional groups protected with appropriate protective groups), the polypeptide chain is extended. Solid phase synthesis methods are largely classified by the tBoc method and the Fmoc method, depending on the type of protective group used. Totally synthetic proteins are disclosed in the literature (Brown A et al., 1996).

The soluble receptors of the present invention can be produced, formulated, administered, or generically used in other alternative forms that can be preferred according to the desired method of use and/or production. The proteins of the invention can be post-translationally modified, for example by glycosylation. The polypeptides or proteins of the invention can be provided in isolated (or purified) biologically active form, or as precursors, derivatives and/or salts thereof. The term “biologically active” meaning that such polypeptides have the ability to reduce the symptoms of MS.

Useful conjugates or complexes can also be generated for improving the agents in terms of drug delivery efficacy. For this purpose, the soluble receptors described herein can be in the form of active conjugates or complex with molecules such as polyethylene glycol and other natural or synthetic polymers (Harris J M and Chess R B, 2003; Greenwald R B et al., 2003; Pillai O and Panchagnula R, 2001). In this regard, the present invention contemplates chemically modified polypeptides and proteins as disclosed herein, in which the polypeptide or the protein is linked with a polymer. Typically, the polymer is water soluble so that the conjugate does not precipitate in an aqueous environment, such as a physiological environment. An example of a suitable polymer is one that has been modified to have a single reactive group, such as an active ester for acylation, or an aldehyde for alkylation. In this way, the degree of polymerization can be controlled. An example of a reactive aldehyde is polyethylene glycol propionaldehyde, or mono-(C1-C10) alkoxy, or aryloxy derivatives thereof (see, for example, Harris, et al., U.S. Pat. No. 5,252,714). The polymer may be branched or unbranched. Moreover, a mixture of polymers can be used to produce the conjugates. The conjugates used for therapy can comprise pharmaceutically acceptable water-soluble polymer moieties. Suitable water-soluble polymers include polyethylene glycol (PEG), monomethoxy-PEG, mono-(C1-C10) alkoxy-PEG, aryloxy- PEG, poly-(N-vinyl pyrrolidone) PEG, tresyl monomethoxy PEG, PEG propionaldehyde, bis-succinimidyl carbonate PEG, propylene glycol homopolymers, a polypropyleneoxide/ethylene oxide co-polymer, polyoxyethylated polyols (e.g., glycerol), polyvinyl alcohol, dextran, cellulose, or other carbohydrate-based polymers. Suitable PEG may have a molecular weight from about 600 to about 60,000, including, for example, 5,000, 12,000, 20,000 and 25,000. A conjugate can also comprise a mixture of such water-soluble polymers.

Examples of conjugates comprise any of the soluble receptor disclosed here above and a polyalkyl oxide moiety attached to the N-terminus of said soluble receptor. PEG is one suitable polyalkyl oxide. As an illustration, any of the soluble receptor disclosed here above can be modified with PEG, a process known as “PEGylation.” PEGylation can be carried out by any of the PEGylation reactions known in the art (see, for example, EP 0 154 316, Delgado et al., Critical Reviews in Therapeutic Drug Carrier Systems 9: 249 (1992), Duncan and Spreafico, Clin. Pharmacokinet. 27: 290 (1994), and Francis et al., Int J Hematol 68: 1 (1998)). For example, PEGylation can be performed by an acylation reaction or by an alkylation reaction with a reactive polyethylene glycol molecule. In an alternative approach, conjugates are formed by condensing activated PEG, in which a terminal hydroxy or amino group of PEG has been replaced by an activated linker (see, for example, Karasiewicz et al., U.S. Pat. No. 5,382,657). Preferably, all these modifications do not affect significantly the ability of the soluble receptor to reduce the symptoms of MS.

The soluble receptors here above described may comprise an additional N-terminal amino acid residue, preferably a methionine. Indeed, depending on the expression system and conditions, polypeptides may be expressed in a recombinant host cell with a starting Methionine. This additional amino acid may then be either maintained in the resulting recombinant protein, or eliminated by means of an exopeptidase, such as Methionine Aminopeptidase, according to methods disclosed in the literature (Van Valkenburgh H A and Kahn R A, Methods Enzymol. (2002) 344:186-93; Ben-Bassat A, Bioprocess Technol. (1991) 12:147-59).

### 8) Pharmaceutical Uses of the Soluble IL-18Rα of the Present Invention:

In a particular aspect, the present invention pertains to any of the above or below described soluble IL-18Rα for use as a medicament. Preferably, any of the above or below described soluble IL-18Rα have the ability to reduce the symptoms of an autoimmune or demyelinating disease, in particular MS. Therefore, preferably, all the modifications to soluble IL-18Rα described herein do not affect significantly their ability to reduce the symptoms of MS. Even more preferably, the modifications to soluble IL-18Rα described herein enhance their ability to reduce the symptoms of MS (e.g. by enhancing their half life etc. . . . ).

The invention also pertains to methods for treating, preventing or ameliorating the symptoms of MS in a human subject by administering an effective amount of a soluble IL-18Rα to the subject. The methods of the present invention include administering a soluble IL-18Rα as described herein to an individual afflicted with MS, for a period of time sufficient to induce a sustained improvement in the patient's condition. The invention also provides, in part, the use of a soluble IL-18Rα in the manufacture of a medicament for the treatment of MS. In some embodiments, the soluble IL-18Rα are the one disclosed here above. In some embodiments, the disease to treat is relapsing-remitting (RR) MS, secondary progressive (SP) MS, primary progressive (PP) MS or progressive relapsing (PR) MS.

Basis, in part, for the invention are the results disclosed here above and in the examples of the present application. These results strongly support the use of soluble IL-18Rα in the treatment of MS. The subject methods involve administering to the patient a soluble IL-18Rα that is capable of reducing the effective amount of endogenous biologically active IL-18Rα, such as by preventing its biological activity. Such soluble IL-18Rα include the one disclosed here above.

In one preferred embodiment of the invention, sustained-release forms of the soluble IL-18Rα described here above are used. Sustained-release forms suitable for use in the disclosed methods include, but are not limited to, soluble IL-18Rα that are encapsulated in a slowly-dissolving biocompatible polymer, admixed with such a polymer, and or encased in a biocompatible semi-permeable implant. Degradable polymer microspheres have been designed to maintain high systemic levels of therapeutic proteins. Microspheres are prepared from degradable polymers such as poly(lactide-co-glycolide) (PLG), polyanhydrides, poly (ortho esters), nonbiodegradable ethylvinyl acetate polymers, in which proteins are entrapped in the polymer (Gombotz and Pettit, Bioconjugate Chem. 6:332 (1995); Ranade, “Role of Polymers in Drug Delivery,” in Drug Delivery Systems, Ranade and Hollinger (eds.), pages 51-93 (CRC Press 1995); Roskos and Maskiewicz, “Degradable Controlled Release Systems Useful for Protein Delivery,” in Protein Delivery: Physical Systems, Sanders and Hendren (eds.), pages 45-92 (Plenum Press 1997); Bartus et al., Science 281:1161 (1998); Putney and Burke, Nature Biotechnology 16:153 (1998); Putney, Curr. Opin. Chem. Biol. 2:548 (1998)). Polyethylene glycol (PEG)-coated nanospheres can also provide carriers for intravenous administration of therapeutic proteins (see, for example, Gref et al., Pharm. Biotechnol. 10:167 (1997)). In addition, the soluble IL-18Rα can be conjugated with polyethylene glycol (pegylated) to prolong its serum half-life or to enhance protein delivery.

To treat MS, the soluble IL-18Rα, and in particular the soluble IL-18Rα disclosed here above, is administered to the patient in an amount and for a time sufficient to induce a sustained improvement in at least one indicator that reflects the severity of the disorder. The degree of improvement is determined based on signs or symptoms, and may also employ questionnaires that are administered to the patient, such as quality-of-life questionnaires. A therapeutically effective amount of a soluble IL-18Rα, is that sufficient to achieve such a sustained improvement.

Improvement might be induced by repeatedly administering a dose of soluble IL-18Rα until the patient manifests an improvement over baseline for the chosen indicator or indicators. Although the extent of the patient's illness after treatment may appear improved according to one or more indicators, treatment may be continued indefinitely at the same level or at a reduced dose or frequency. Once treatment has been reduced or discontinued, it later may be resumed at the original level of symptoms should reappear.

The pharmaceutical compositions used in the methods of the present invention may contain, in combination with the soluble IL-18Rα as active ingredient, suitable pharmaceutically acceptable diluents, carriers, biologically compatible vehicles and additives which are suitable for administration to an animal (for example, physiological saline solution) and optionally comprising auxiliaries (like excipients, stabilizers, or adjuvants) which facilitate the processing of the active compounds into preparations which can be used pharmaceutically. The pharmaceutical compositions may be formulated in any acceptable way to meet the needs of the mode of administration. For example, the use of biomaterials and other polymers for drug delivery, as well the different techniques and models to validate a specific mode of administration, are disclosed in literature (Luo B and Prestwich G D, 2001; Cleland J L et al., Curr Opin Biotechnol. (2001), 12(2):212-9). “Pharmaceutically acceptable” is meant to encompass any carrier, which does not interfere with the effectiveness of the biological activity of the active ingredient and that is not toxic to the host to which is administered. For example, for parenteral administration, the above active ingredients may be formulated in unit dosage form for injection in vehicles such as saline, dextrose solution, serum albumin and Ringer's solution. Carriers can be selected also from starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the various oils, including those of petroleum, animal, vegetable or synthetic origin (peanut oil, soybean oil, mineral oil, sesame oil).

The pharmaceutical composition may be in a liquid or lyophilized form and comprises a diluent (Tris, citrate, acetate or phosphate buffers) having various pH values and ionic strengths, solubilizer such as Tween or Polysorbate, carriers such as human serum albumin or gelatin, preservatives such as thimerosal, parabens, benzylalconium chloride or benzyl alcohol, antioxidants such as ascrobic acid or sodium metabisulfite, and other components such as lysine or glycine. Selection of a particular composition will depend upon a number of factors, including the condition being treated, the route of administration and the pharmacokinetic parameters desired. A more extensive survey of components suitable for pharmaceutical compositions is found in Remington's Pharmaceutical Sciences, 18th ed. A. R. Gennaro, ed. Mack, Easton, Pa. (1980).

In a preferred embodiment, soluble IL-18Rα is administered in the form of a physiologically acceptable composition comprising purified recombinant protein in conjunction with physiologically acceptable carriers, excipients or diluents. Such carriers are non toxic to recipients at the dosages and concentrations employed. Ordinarily, preparing such compositions entails combining the soluble IL-18Rα with buffers, antioxidants such as ascorbic acid, low molecular weight polypeptides (such as those having fewer than 10 amino acids), proteins, amino acids, carbohydrates such as glucose, sucrose or dextrins, cheating agents such as EDTA, glutathione and other stabilizers and excipients. Neutral buffered saline or saline mixed with conspecific serum albumin are exemplary appropriate diluents. The soluble IL-18Rα is preferably formulated as a lyophilizate using appropriate excipient solutions (e.g., sucrose) as diluents. Appropriate dosages can be determined in standard dosing trials, and may vary according to the chosen route of administration. In accordance with appropriate industry standards, preservatives may also be added, such as benzyl alcohol. The amount and frequency of administration will depend, of course, on such factors as the severity of the indication being treated, the desired response, the age and condition of the patient, and so forth.

Any accepted mode of administration can be used and determined by those skilled in the art to establish the desired blood levels of the active ingredients. For example, administration may be by various parenteral routes such as subcutaneous, intravenous, intradermal, intramuscular, intraperitoneal, intranasal, transdermal, rectal, oral, or buccal routes. Preferably the pharmaceutical compositions of the invention are administered by injection, either subcutaneous or intravenous. The route of administration eventually chosen will depend upon a number of factors and may be ascertained by one skilled in the art.

The pharmaceutical compositions used in the methods of the present invention can also be administered in sustained or controlled release dosage forms, including depot injections, osmotic pumps, and the like, for the prolonged administration of the soluble IL-18Rα at a predetermined rate, preferably in unit dosage forms suitable for single administration of precise dosages.

Parenteral administration can be by bolus injection or by gradual perfusion over time. Preparations for parenteral administration include sterile aqueous or non-aqueous solutions, suspensions, and emulsions, which may contain auxiliary agents or excipients known in the art, and can be prepared according to routine methods. In addition, suspension of the active compounds as appropriate oily injection suspensions may be administered. Suitable lipophilic solvents or vehicles include fatty oils, for example, sesame oil, or synthetic fatty acid esters, for example, sesame oil, or synthetic fatty acid esters, for example, ethyl oleate or triglycerides. Aqueous injection suspensions that may contain substances increasing the viscosity of the suspension include, for example, sodium carboxymethyl cellulose, sorbitol, and/or dextran. Optionally, the suspension may also contain stabilizers. Pharmaceutical compositions include suitable solutions for administration by injection, and contain from about 0.01 to 99.99 percent, preferably from about 20 to 75 percent of active compound together with the excipient.

It is understood that the dosage administered will be dependent upon the age, sex, health, and weight of the recipient, kind of concurrent treatment, if any, frequency of treatment, and the nature of the effect desired. The dosage will be tailored to the individual subject, as is understood and determinable by one of skill in the art. The total dose required for each treatment may be administered by multiple doses or in a single dose.

In one embodiment of the invention, the soluble IL-18Rα disclosed here above is administered one time per week to treat MS, in another embodiment is administered at least two times per week, and in another embodiment is administered at least once per day. If injected, the effective amount, per adult (a person who is 18 years of age or older) dose, of a soluble IL-18Rα as defined here above, ranges from 1-200 mg/m², or from 1-40 mg/m² or about 5-25 mg/m². Alternatively, a flat dose may be administered, whose amount may range from 2-400 mg/dose, 2-100 mg/dose or from about 10-80 mg/dose. If the dose is to be administered more than one time per week, an exemplary dose range is the same as the foregoing described dose ranges or lower. Preferably, such soluble IL-18Rα is administered two or more times per week at a per dose range of 25-100 mg/dose. In one embodiment of the invention, MS is treated by administering a preparation acceptable for injection containing a soluble IL-18Rα, as defined here above, at 80-100 mg/dose, or alternatively, containing 80 mg per dose.

If a route of administration of the soluble IL-18Rα other than injection is used, the dose is appropriately adjusted in accord with standard medical practices. For example, if the route of administration is inhalation, dosing may be one to seven times per week at dose ranges from 10 mg/dose to 50 mg per dose.

In many instances, an improvement in a patient's condition will be obtained by injecting a dose of up to about 100 mg of the soluble IL-18Rα as disclosed hereabove, one to three times per week over a period of at least three weeks, though treatment for longer periods may be necessary to induce the desired degree of improvement. The regimen may be continued indefinitely.

### 9) Combination Therapy:

In some embodiments, a soluble IL-18Rα as defined here above, is administered in conjunction with a second therapeutic agent for treating or preventing MS. For example, a soluble IL-18Rα may be administered in conjunction with any of the standard treatments for MS including, e.g., corticosteroïds, immunosuppressive drugs, neuro-protective agents, immunomodulatory drugs or interferons.

In an embodiment of the present invention, a soluble IL-18Rα as defined here above is administered in conjunction with a corticosteroïd. By “corticosteroid” is meant any naturally occurring or synthetic steroid hormone which can be derived from cholesterol and is characterized by a hydrogenated cyclopentanoperhydrophenanthrene ring system. Naturally occurring corticosteriods are generally produced by the adrenal cortex. Synthetic corticosteriods may be halogenated. Corticosteroids may have glucocorticoid and/or mineralocorticoid activity.

Exemplary corticosteroids include, for example, dexamethasone, betamethasone, triamcinolone, triamcinolone acetonide, triamcinolone diacetate, triamcinolone hexacetonide, beclomethasone, dipropionate, beclomethasone dipropionate monohydrate, flumethasone pivalate, diflorasone diacetate, fluocinolone acetonide, fluorometholone, fluorometholone acetate, clobetasol propionate, desoximethasone, fluoxymesterone, fluprednisolone, hydrocortisone, hydrocortisone acetate, hydrocortisone butyrate, hydrocortisone sodium phosphate, hydrocortisone sodium succinate, hydrocortisone cypionate, hydrocortisone probutate, hydrocortisone valerate, cortisone acetate, paramethasone acetate, methylprednisolone, methylprednisolone acetate, methylprednisolone sodium succinate, prednisolone, prednisolone acetate, prednisolone sodium phosphate, prednisolone tebutate, clocortolone pivalate, flucinolone, dexamethasone 21-acetate, betamethasone 17-valerate, isoflupredone, 9-fluorocortisone, 6-hydroxydexamethasone, dichlorisone, meclorisone, flupredidene, doxibetasol, halopredone, halometasone, clobetasone, diflucortolone, isoflupredone acetate, fluorohydroxyandrostenedione, beclomethasone, flumethasone, diflorasone, fluocinolone, clobetasol, cortisone, paramethasone, clocortolone, prednisolone 21-hemisuccinate free acid, prednisolone metasulphobenzoate, prednisolone terbutate, and triamcino lone acetonide 21-palmitate.

Preferred examples of corticosteroids administered in conjunction with a soluble IL-18Rα as defined here above are prednisone and/or IV methylprednisolone.

In an embodiment of the present invention, a soluble IL-18Rα as defined here above is administered in conjunction with an immunosuppressive drug. In an embodiment of the present invention, the immunosuppressive drug is chosen in the group consisting of methotrexate, azathioprine, cyclophosphamide, and cladribine, which are generally used for severe progressive forms of demyelinating diseases.

In another embodiment of the present invention, a soluble IL-18Rα as defined here above is administered in conjunction with a neuroprotective agent. In an embodiment of the present invention, the neuroprotective agent is chosen in the group consisting of oral myelin, Copaxone (Glatiramer Acetate from Teva), Tysabri (Biogen/Elan), Novantrone (Serono), Teriflunomide (Aventis), Cladribine (Serono/IVAX), 683699 (T-0047) of GSK/Tanabe Seiyaku, Daclizumab (Roche), Laquinimod (Active Biotech) and ZK-117137 (Schering AG). These compounds are all on the market or in clinical trials to treat MS.

In another embodiment of the present invention, a soluble IL-18Rα as defined here above is administered in conjunction with an immunomodulatory drug. In this respect, a particular immunomodulatory drug for use in the present invention include FTY720 (2-amino-2-[2-(4-octylphenyl)ethyl]-1,3-propanediol, fingolimod). FTY720 which is in phase II to treat MS (Novartis) has the following formula:

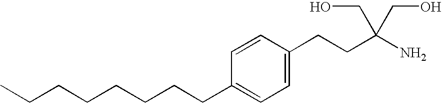

FTY720 has been identified as an orally active immunosuppressant (see, e.g., WO 94/08943; WO 99/36065) obtained by chemical modification of myriocin. Other immunomodulatory drugs for use in the present invention, include derivatives of FTY720. Derivatives of FTY720 include 2-amino-1,3-propanediol compounds as described in WO94/08943, having the following formula, as well as any pharmaceutically acceptable salts thereof:

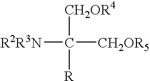

wherein R is an optionally substituted straight- or branched carbon chain which may have, in the chain, a bond, a hetero atom or a group selected from the group consisting of a double bond, a triple bond, oxygen, sulfur, sulfinyl, sulfonyl, —N(R6)- where R6 is hydrogen, alkyl, aralkyl, acyl or alkoxycarbonyl, carbonyl, optionally substituted arylene, optionally substituted cycloalkylene, optionally substituted heteroarylene and an alicycle thereof, and which may be substituted, at the chain end thereof, by a double bond, a triple bond, optionally substituted aryl, optionally substituted cycloalkyl, optionally substituted heteroaryl or an alicycle thereof, an optionally substituted aryl, an optionally substituted cycloalkyl, an optionally substituted heteroaryl or an alicycle thereof, and

R2, R3, R4 and R5 are the same or different and each represents a hydrogen, an alkyl, an aralkyl, an acyl or an alkoxycarbonyl or, R4 and R5 may be bonded to form an alkylene chain which may be substituted by an alkyl, aryl or aralkyl.

The above, optionally substituted straight- or branched carbon chains, may have a substituent selected from the group consisting of alkoxy, alkenyloxy, alkynyloxy, aralkyloxy, alkylenedioxy, acyl, alkylamino, alkylthio, acylamino, alkoxycarbonyl, alkoxycarbonylamino, acyloxy, alkylcarbamoyl, haloalkyl, haloalkoxy, nitro, halogen, amino, hydroxyimino, hydroxy, carboxy, optionally substituted aryl, optionally substituted aryloxy, optionally substituted cycloalkyl, optionally substituted heteroaryl and an alicycle thereof, the aforementioned optionally substituted arylene, optionally substituted cycloalkylene, optionally substituted heteroarylene and an alicycle thereof may have a substituent selected from the group consisting of alkoxy, alkenyloxy, alkynyloxy, aralkyloxy, alkylenedioxy, acyl, alkylamino, alkylthio, acylamino, alkoxycarbonyl, alkoxycarbonylamino, acyloxy, alkylcarbamoyl, haloalkyl, haloalkoxy, nitro, halogen, amino, hydroxy and carboxy; and the optionally substituted aryl, optionally substituted aryloxy, optionally substituted cycloalkyl, optionally substituted heteroaryl and an alicycle thereof may have a substituent selected from the group consisting of alkyl, alkoxy, alkenyloxy, alkynyloxy, aralkyloxy, alkylenedioxy, acyl, alkylamino, alkylthio, acylamino, alkoxycarbonyl, alkoxycarbonylamino, acyloxy, alkylcarbamoyl, haloalkyl, haloalkoxy, nitro, halogen, amino, hydroxy and carboxy.

Specific examples of such 2-amino-1,3-propanediol compounds include 2-amino-2-[2-(4-heptylphenyl)ethyl]-1,3-propanediol, 2-amino-2-[2-(4-octylphenyl)ethyl]-1,3-propanediol, 2-amino-2-[2-(4-nonylphenyl)ethyl]-1,3-propanediol 2-amino-2-[2-(4-decylphenyl)ethyl]-1,3-propanediol, 2-amino-2-[2-(4-undecylphenyl)ethyl]-1,3-propanediol, 2-amino-2-[2-(4-dodecylphenyl)ethyl]-1,3-propanediol, 2-amino-2-[2-(4-tridecylphenyl)-ethyl]-1,3-propanediol, 2-amino-2-[2-(4-tetradecylphenyl)ethyl]-1,3-propanediol, 2-amino-2-[2-(4-hexyloxyphenyl)ethyl]-1,3-propanediol, 2-amino-2-[2-(4-heptyloxyphenyl)ethyl]-1,3-propanediol, 2-amino-2-[2-(4-octyloxyphenyl)ethyl]-1,3-propanediol, 2-amino-2-[2-(4-nonyloxyphenyl)ethyl]-1,3-propanediol, 2-amino-2-[2-(4-decyloxyphenyl)ethyl]-1,3-propanediol, 2-amino-2-[2-(4-undecyloxyphenyl)ethyl]-1,3-propanediol, 2-amino-2-[2-(4-dodecyloxyphenyl)ethyl]-1,3-propanediol, 2-amino-2-[2-(4-tridecyloxyphenyl)ethyl]-1,3-propanediol, 2-amino-2-[2-(4-(8-fluorooctyl)phenyl)ethyl]-1,3-propanediol, 2-amino-2-[2-(4-(12-fluorododecyl)phenyl)ethyl]-1,3-propanediol, 2-amino-2-[2-(4-(7-fluoroheptyloxy)phenyl)ethyl]-1,3-propanediol, 2-amino-2-[2-(4-(11-fluoroundecyloxy)phenyl)ethyl]-1,3-propanediol, 2-amino-2-[2-(4-(7-octenyloxy)phenyl)ethyl]-1,3-propanediol, 2-amino-2-[2-(4-heptylphenyl)ethyl]-1,3-propanediol, 2-amino-2-[2-(4-octylphenyl)ethyl]-1,3-propanediol, 2-amino-2-[2-(4-nonylphenyl)ethyl]-1,3-propanediol, 2-amino-2-[2-(4-decylphenyl)ethyl]-1,3-propanediol, 2-amino-2-[2-(4-undecylphenyl)ethyl]-1,3-propanediol, 2-amino-2-[2-(4-dodecylphenyl)ethyl]-1,3-propanediol, 2-amino-2-[2-(4-heptyloxyphenyl)ethyl]-1,3-propanediol, 2-amino-2-[2-(4-octyloxyphenyl)ethyl]-1,3-propanediol, 2-amino-2-[2-(4-nonyloxyphenyl)ethyl]-1,3-propanediol, 2-amino-2-[2-(4-undecyloxyphenyl)ethyl]-1,3-propanediol, or 2-amino-2-[2-(4-(7-octenyloxy)phenyl)ethyl]-1,3-propanediol, as well as any pharmaceutically acceptable salts thereof.

In another embodiment of the present invention, a soluble IL-18Rα as defined here above is administered in conjunction with an interferon. In this respect, a particular interferon for use in the present invention is interferon-beta. The terms “interferon (IFN)” and “interferon-beta (IFN-beta)”, as used herein, are intended to include fibroblast interferon in particular of human origin, as obtained by isolation from biological fluids or as obtained by DNA recombinant techniques from prokaryotic or eukaryotic host cells, as well as their salts, functional derivatives, variants, analogs and active fragments. A particular type of interferon beta is interferon beta-1a.

The use of interferons of human origin is preferred in accordance with the present invention. IFN-beta suitable in accordance with the present invention is commercially available, e.g., as Rebif® (Serono), Avonex® (Biogen) or Bertaseron/Betaferon® (Schering). Rebif® (recombinant human interferon-) is the latest development in interferon therapy for multiple sclerosis (MS) and represents a significant advance in treatment. Rebif® is interferon (IFN)-beta 1a, produced from mammalian cell lines. It was established that interferon beta-1a given subcutaneously three times per week is efficacious in the treatment of Relapsing-Remitting Multiple Sclerosis (RRMS). Interferon beta-1a can have a positive effect on the long-term course of MS by reducing number and severity of relapses and reducing the burden of the disease and disease activity as measured by MRI. Particular examples of interferon administered in conjunction with soluble IL-18Rα for use in the methods of the present invention therefore are Rebif® (Serono), Avonex® (Biogen) or Bertaseron/Betaferon® (Schering).

A particular aspect of the invention pertains to a method of treating MS, particularly relapsing-remitting (RR) MS, secondary progressive (SP) MS, primary progressive (PP) MS or progressive relapsing (PR) MS, in a subject in need of such treatment, comprising administering to the subject a therapeutically effective amount of a combination of a soluble IL-18Rα as disclosed here above and a corticosteroïd, an immunosuppressive drug, a neuro-protective agent, an immunomodulatory drug or an interferon as disclosed here above. In certain embodiments the cortisteroid is prednisone or IV methylprednisolone. In certain embodiments the immunosuppressive drug is methotrexate, azathioprine, cyclophosphamide or cladribine. In certain embodiments the neuroprotective agent is oral myelin, Copaxone, Tysabri, Novantrone, Teriflunomide, Cladribine, 683699 (T-0047), Daclizumab, Laquinimod or ZK-117137. In certain embodiments the immunomodulatory drug is 2-amino-2-[2-(4-octylphenyl)ethyl]-1,3-propanediol (FTY720). In certain embodiments the interferon is interferon beta-1a (in particular Rebif® (Serono)).

The soluble IL-18Rα as defined here above and the second therapeutic agent as disclosed here above may be administered simultaneously, separately or sequentially. For example, the soluble IL-18Rα may be administered first, followed by the second therapeutic agent. Alternatively, the second therapeutic agent may be administered first, followed by the soluble IL-18Rα. In some cases, the soluble IL-18Rα and the second therapeutic agent are administered in the same formulation. In other cases the soluble IL-18Rα and the second therapeutic agent are administered in different formulations. When the soluble IL-18Rα and the second therapeutic agent are administered in different formulations, their administration may be simultaneous or sequential.

The invention further pertains to product comprising any of the above or below described soluble IL-18Rα, and a corticosteroïd, immunosuppressive drug, neuro-protective agent, immunomodulatory drug or interferon, as disclosed here above, as a combined preparation for simultaneous, separate or sequential use in the therapy of MS in a mammalian subject, preferably a human subject.

All patent and literature references cited in the present specification are hereby incorporated by reference in their entirety.

Further aspects and advantages of the present invention will be disclosed in the following examples, which should be considered as illustrative only, and do not limit the scope of this application.

### EXAMPLES

### Example 1

### p35_{-/-} IL-18_{-/-} Double Knockout Mice are Susceptible to EAE

It has previously been shown that deletion of IL-12p35, renders mice hypersusceptible to MOG (myelin oligodendrocyte glycoprotein)-peptide-induced Experimental Autoimmune Encephalomyelitis (EAE) in mice (Becher, B., et al. J. Clin. Invest 110, 493-497 (2002)). IL-18 acts in synergy with IL-12 to polarize Th1 cells (type 1 helper T cells) and Shi et al. have produced evidence demonstrating that mice deficient in IL-18 are resistant to EAE (Shi, F. D., et al., J. Immunol. 165, 3099-3104 (2000)).

To assess whether IL-18 is capable of compensating for the loss of IL-12 in p35_{-/-} mice, thus leading to their EAE susceptibility, we generated mice deficient in both IL-12p35 and IL-18 (p35_{-/-}X IL-18_{-/-}).

Mice (n=5 mice/group) were immunized subcutaneously with 200 μg of MOG₃₅₋₅₅ peptide (amino acid sequence: MEVGWYRSPFSRVVHLYRNGK (SEQ ID NO: 11)), obtained from GenScript, emulsified in CFA (DIFCO, Detroit, Mich.). Mice received 200 ng pertussis toxin (Sigma-Aldrich) intraperitoneally at the time of immunization and 48 hours later.

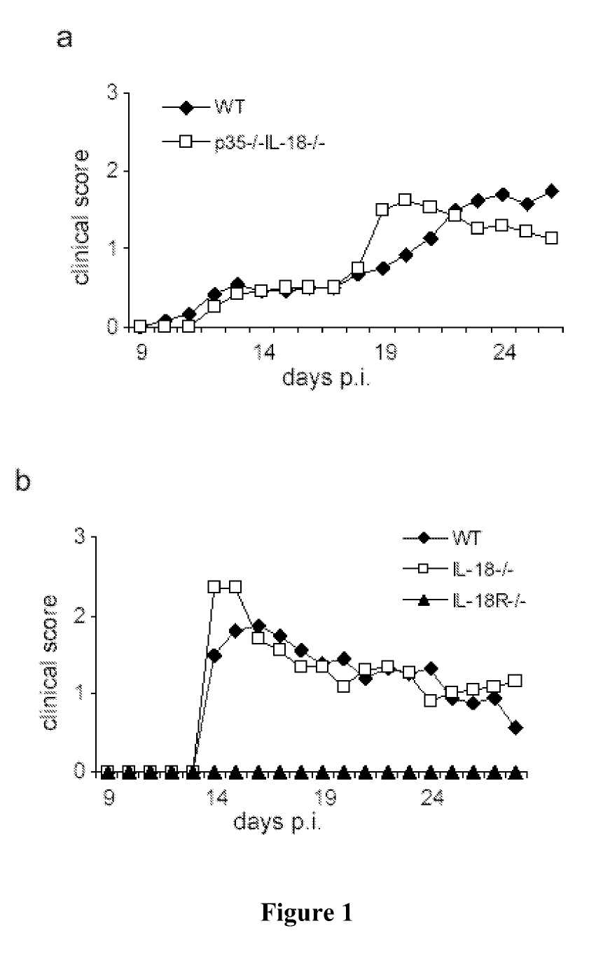

Mice were scored daily as follows: 0) no detectable signs of EAE; 0.5) distal tail limp; 1) complete tail limp; 2) unilateral partial hind limb paralysis; 2.5) bilateral partial limb paralysis; 3) complete bilateral hind limb paralysis; 3.5) complete hind limb paralysis and unilateral forelimb paralysis; 4) total paralysis of fore and hind limbs (score >4 to be euthanized); 5) death. Each time point shown is the average disease score of each group. Statistical significance was assessed using an unpaired Student's t-Test. Immunization with MOG₃₅₋₅₅ emulsified in CFA showed that p35_{-/-} x IL-18_{-/-} mice are fully susceptible to EAE and have a similar disease score and development as is produced in wt (see FIG. 1a). Therefore, the lack of protection generated by IL-18 deletion in p35_{-/-} mice shows that IL-18 is not responsible for inducing EAE susceptibility in p35_{-/-} mice but it also implies that IL-18 itself is a cytokine that has little or no effect in EAE pathogenesis.

### Example 2

### IL-18_{-/-}, but not IL-18Rα_{-/-}, Mice are Susceptible to EAE

As our experiments in p35_{-/-} x IL-18_{-/-} mice seemed to contradict the previously proposed pathogenic role for IL-18 in EAE, we actively immunized wt and IL-18_{-/-} mice with MOG peptide (as described in example 1) and found that IL-18_{-/-} mice were fully susceptible to EAE and indeed had a clinical score and disease progression comparable to that of the wt mice (see FIG. 1b and Table 1).

Mice deficient in IL-18Rα have been described as having a phenotype similar to that of IL-18_{-/-} mice in that IFNγ production is reduced. Interestingly, and in sharp contrast to both wt and IL-18_{-/-} mice, IL-18Rα_{-/-} mice were completely resistant to EAE induction (see FIG. 1b and Table 1).

Histological analysis of the spinal cords from wt. IL-18_{-/-} and IL-18Rα_{-/-} mice obtained after EAE induction demonstrated that leukocyte infiltration into the CNS correlated well with clinical severity of disease.

To do so, mice were euthanized with CO2, followed by perfusion with PBS and subsequent perfusion with 4% paraformaldehyde (PFA) in PBS. The spinal column was removed and fixed in 4% PFA in PBS. The spinal cord was then dissected and paraffin-embedded prior to staining with either haematoxylin & eosin or CD3, B220 and MAC-3 antibodies (BD Pharmingen) to assess infiltration of inflammatory cells or luxol fast blue to determine the degree of demyelination.

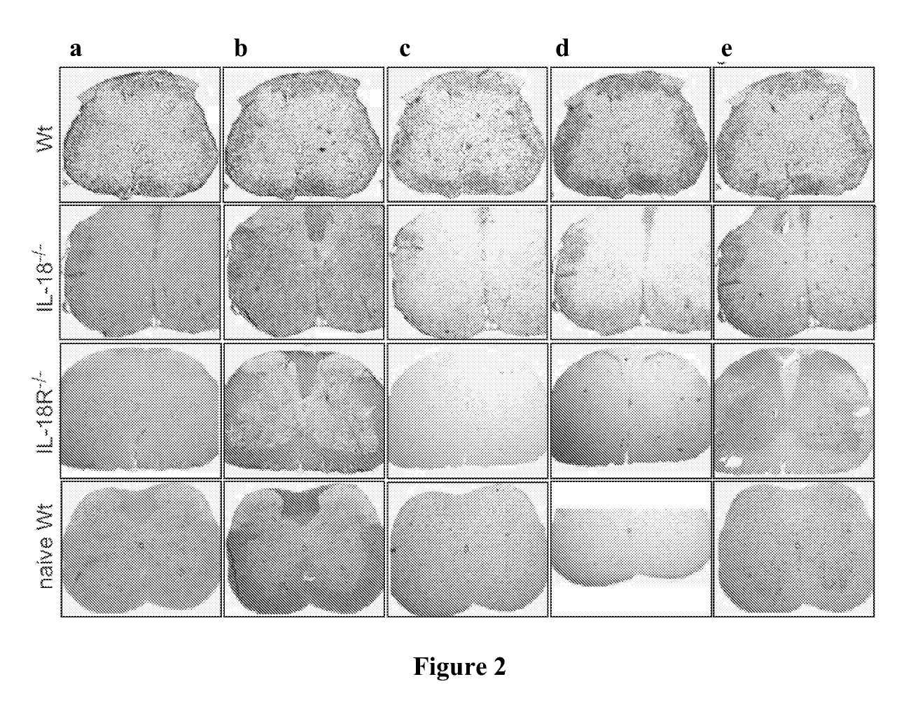

EAE-susceptible wt and IL-18_{-/-} mice had significant inflammation, characterized by infiltration of inflammatory cells (FIG. 2a) such as T cells (FIG. 2c), macrophages (FIG. 2e) and B cells (FIG. 2d), and demyelination (FIG. 2b), while there was no presence of inflammatory infiltrates or demyelination in the spinal cord of EAE-resistant IL-18Rα_{-/-} mice (FIG. 2a-e).

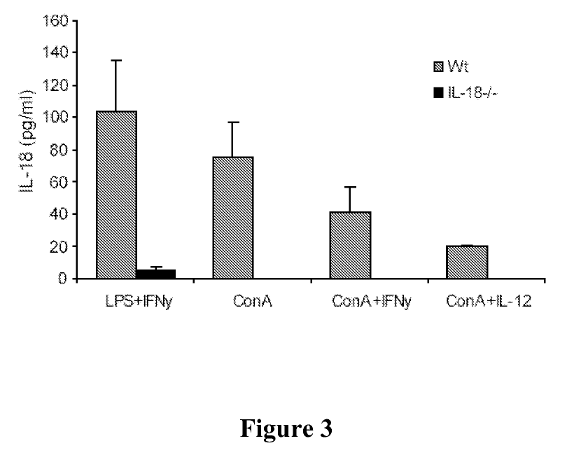

To verify the inability of IL-18_{-/-} mice to secrete IL-18, we extensively verified the targeting strategy and genotype of the mice and could clearly establish that IL-18_{-/-} mice do not contain IL-18 mRNA or protein. We also analyzed whether we could detect IL-18 secreted from activated splenocytes derived from wt and IL-18_{-/-} mice, which showed that IL-18_{-/-} mice are indeed completely IL-18 deficient in contrast to wt mice (See FIG. 3).

As it has been observed in many experimental systems that deletion of IL-18 consistently results in the paucity of an IFNγ response (Wei, X. Q. et al. J. Immunol. 163, 2821-2828 (1999), Kinjo, Y. et al. J. Immunol. 169, 323-329 (2002)), we stimulated lymphocytes derived from naïve wt. IL-18_{-/-} and IL-18Rα_{-/-} mice in vitro with the lectin Concanavalin A (ConA) for 16 hours and IFN-γ production was subsequently measured by ELISA.

To do so, axillary and inguinal lymph nodes (LN) were isolated from naïve mice. 2×10⁵ cells were placed as triplicates in a 96-well plate. 5 μg/ml ConA was used for stimulation for 16 hours and IFN-γ production was subsequently measured by ELISA (Pharmingen, La Jolla, Calif.).

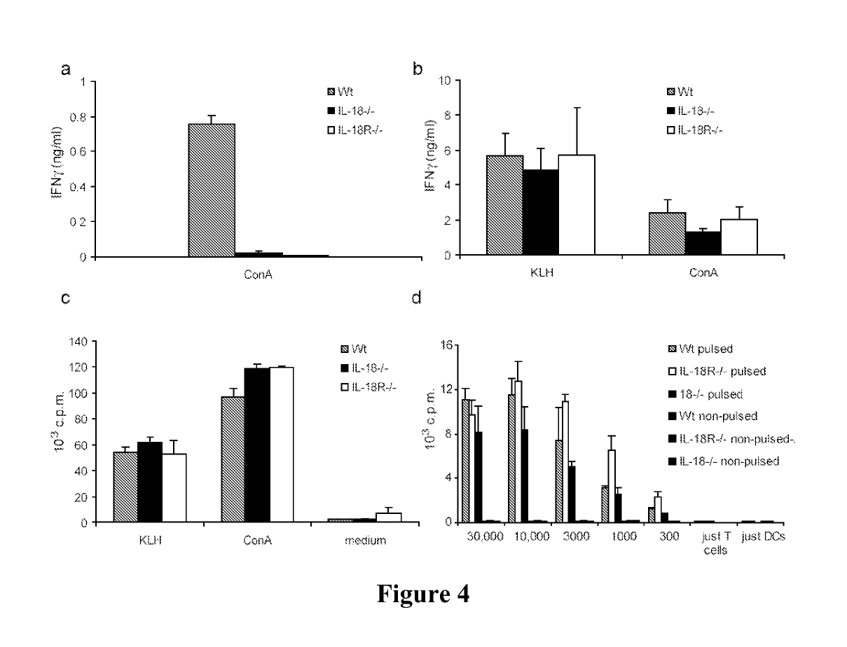

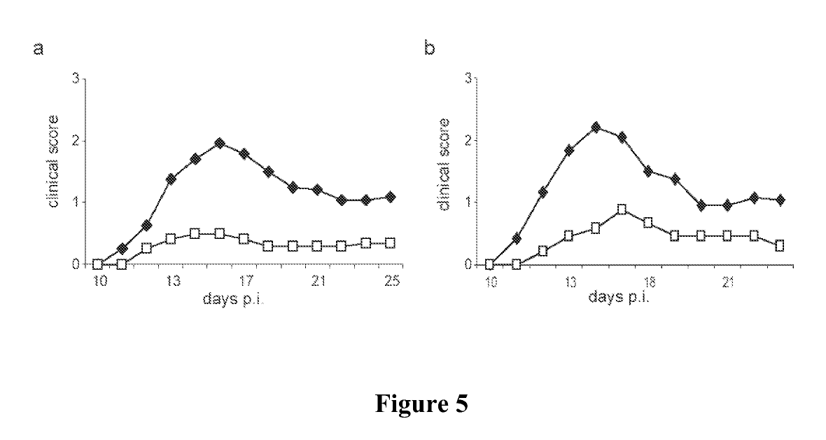

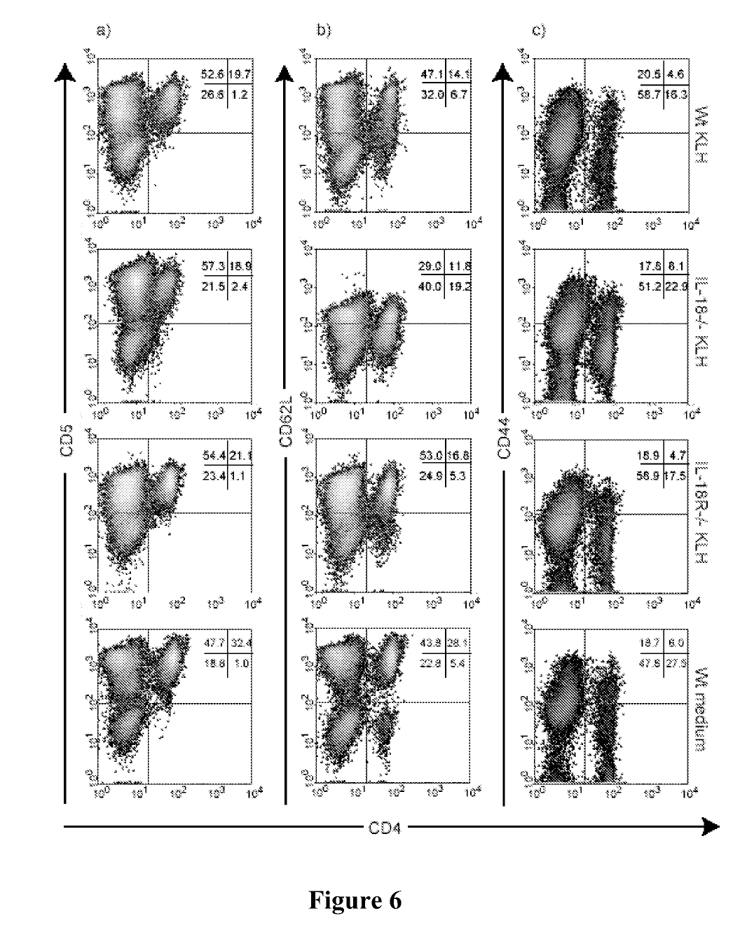

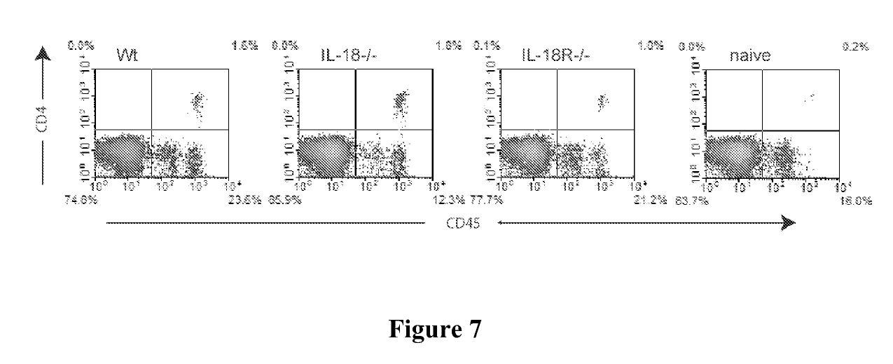

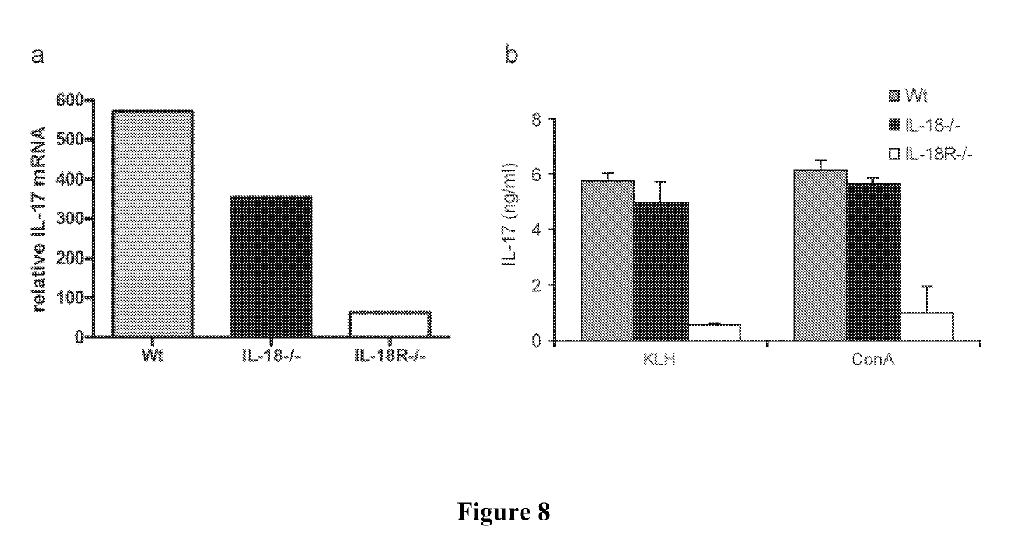

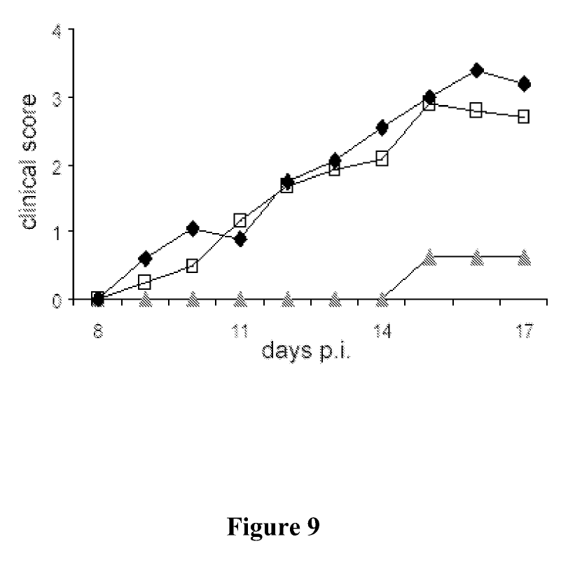

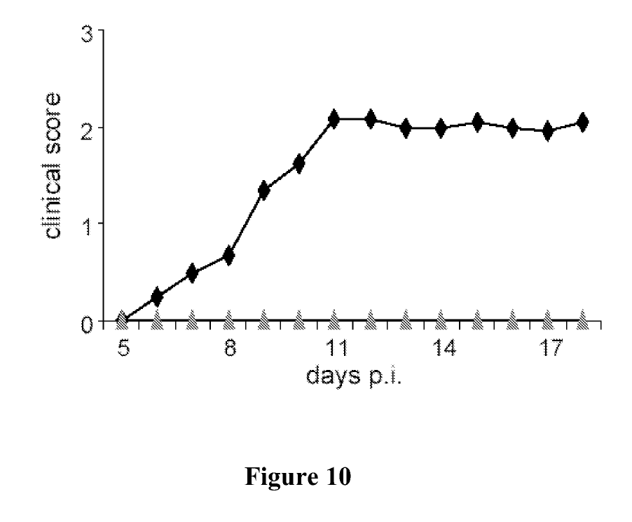

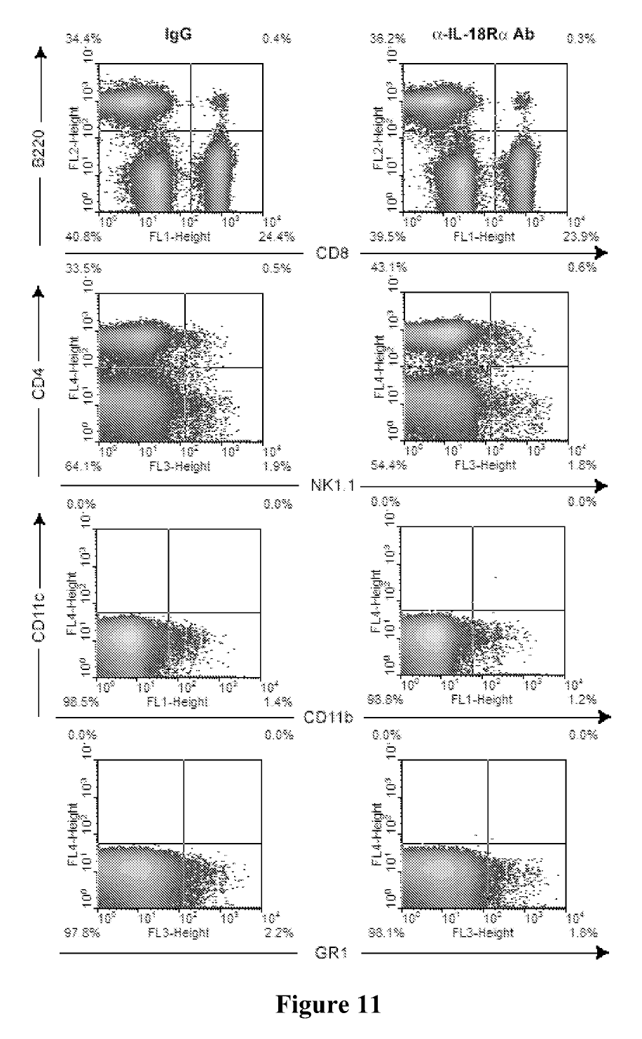

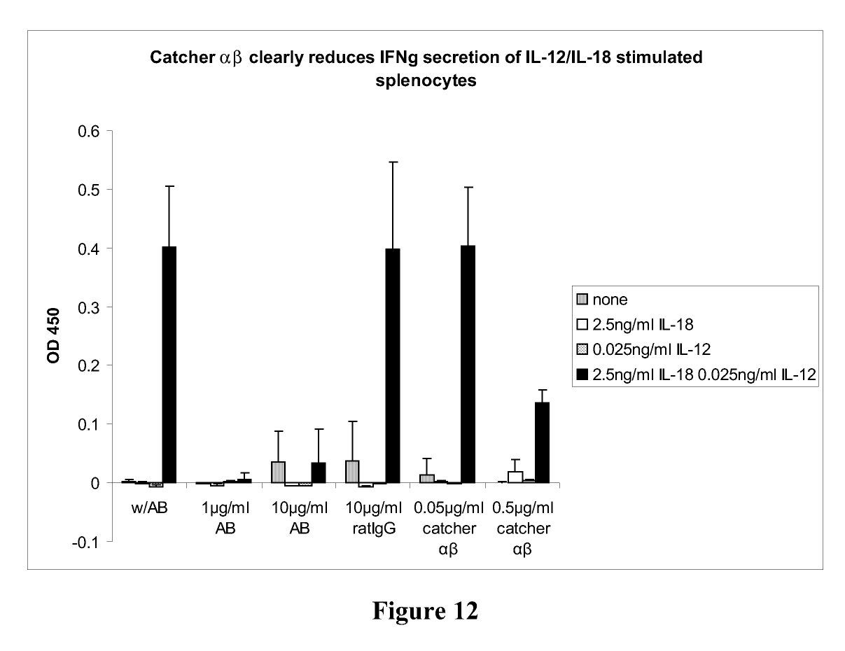

Consistent with the principle that IL-18 has an effect on IFNγ production, LN cells from both IL-18_{-/-} and IL-18Rα_{-/-} mice did not secrete IFNγ in contrast to the wt LN cells (FIG. 4a).

### Example 3

### Blocking IL-18Rα Prevents EAE in IL-18_{-/-} Mice

The discordant behavior of IL-18- and IL-18Rα-deficient mice with regards to EAE strongly points towards an additional IL-18Rα ligand with powerful encephalitogenic properties. In order to assess whether IL-18Rα and IL-18 have independent biological functions, we blocked IL-18Rα in EAE-susceptible IL-18_{-/-} mice.

Mice (n=5 mice/group) were immunized subcutaneously with 200 μg of MOG₃₅₋₅₅ peptide (amino acid sequence: MEVGWYRSPFSRVVHLYRNGK (SEQ ID NO: 11)), obtained from GenScript, emulsified in CFA (DIFCO, Detroit, Mich.). Mice received 200 ng pertussis toxin (Sigma-Aldrich) intraperitoneally at the time of immunization and 48 hours later. Monoclonal anti-IL-18Rα antibody (clone 112624) (R&D Systems) was or was not administered either 1 day pre-immunization (450 μg/mouse) and every 3 days thereafter (300 μg/mouse) or every 3 days beginning from disease onset (300 μg/mouse).

Mice were scored daily as follows: 0) no detectable signs of EAE; 0.5) distal tail limp; 1) complete tail limp; 2) unilateral partial hind limb paralysis; 2.5) bilateral partial limb paralysis; 3) complete bilateral hind limb paralysis; 3.5) complete hind limb paralysis and unilateral forelimb paralysis; 4) total paralysis of fore and hind limbs (score >4 to be euthanized); 5) death.

Each time point shown is the average disease score of each group. Statistical significance was assessed using an unpaired Student's t-Test.

Treatment of IL-18_{-/-} mice with anti-IL-18Rα mAbs, given 1 day pre-immunization and every 3 days thereafter until the end of the experiment, significantly reduced disease development (FIG. 5a). Administration of anti-IL-18Rα mAbs did not lead to deletion of IL-18Rα-expressing cells nor did it alter the composition of peripheral leukocytes in the blood, LN or spleen (see FIG. 11).

Combining the facts that IL-18Rα antagonists prevent EAE even in mice in which its ligand is completely removed by gene-targeting and that IL-18 has reportedly only a low affinity to IL-18Rα, we propose that another ligand must be responsible for the engagement, signaling and immune development mediated by IL-18Rα.

Interestingly, treating IL-18_{-/-} mice with antagonistic mAbs post-immunization (day 10 p.i.) also abrogated EAE progression (FIG. 5b) and this occurred to the same extent as Abs administered prior to immunization suggesting that IL-18Rα engagement is an important event during the effector phase of EAE.

### Example 4

### Mitogen-, but not Ag-Driven Activation Requires IL-18 for Th1 Polarization

Given the dichotomy between IL-18_{-/-} and IL-18Rα^{-/-} mice with regards to EAE susceptibility, we wanted to determine the ability of both mice to properly prime and polarize naïve T cells towards an effector phenotype. Wt. IL-18^{-/-} and IL-18Rα^{-/-} mice were immunized subcutaneously with KLH and 7 days later lymphocytes were isolated and subsequently restimulated with KLH in vitro.

To do so axillary and inguinal lymph nodes were isolated from mice primed by injections of 100 μg/flank of Keyhole limpit hemocyanin (KLH) (Sigma) emulsified in CFA 7 days earlier. 2×10⁵ cells were placed as triplicates in a 96-well plate. KLH recall cells were stimulated for 48 hours with 50 μg/ml KLH, 5 μg/ml ConA or medium and 0.5 μCi/ml 3[H]-thymidine was added after 24 hours to observe proliferative responses. Thymidine incorporation was assessed using a Filtermate Harvester and a scintillation and luminescence counter. For cytokine analysis, the culture supernatant of sister cultures was harvested after 48 hours and analyzed for IFNγ production by ELISA (Pharmingen, La Jolla, Calif.) and overall cytokine/chemokine secretion by cytokine array (Raybiotech).

Surprisingly, we did not observe any significant difference in the IFNγ-producing ability of IL-18_{-/-} and IL-18Rα_{-/-} mice and the levels of IFNγ produced by lymphocytes derived from IL-18_{-/-} or IL-18Rα_{-/-} mice were identical to that of cells obtained from wt mice (FIG. 4b). Furthermore, the proliferative capacity of Ag-driven lymphocytes between the different mouse strains was identical (FIG. 4c). Our data support the notion that IL-18 is a critical co-factor for the early IFNγ response of freshly polyclonally activated T cells (FIG. 4a) yet Ag-driven Th1 polarization is more dependent on IL-12 alone and thus IL-18 independent.

Although T_{H}1 development appeared unaffected in IL-18R_{α-/-} mice we next wanted to assess the capacity of IL-18Rα-deficient Antigen-Presenting cells (APC's) to prime naïve T cells.

To do so, we co-cultured mature, SMARTA peptide (p11)-pulsed wt. IL-18_{-/-} and IL-18Rα_{-/-} BM (Bone Marrow)-derived Dendritic Cells (DC's) with SMARTA-TcR-transgenic CD4₊ T cells and measured proliferation by thymidine incorporation (FIG. 4d).

The protocol used was the following:

Generation of BM-derived DC's: BM-donor mice were euthanized using CO₂ and femur and tibia were removed. BM-cells were isolated by flushing the bones with PBS and were filtered through a 100 μm cell strainer. Cells (2-2.5×10⁶ in 10 ml) were cultured in complete RPMI with the addition of 10% GM-CSF. After at least 6 days, BM-derived DC's were matured with 10 μg/ml lipopolysaccharide (LPS) overnight while immature BM-derived DC's are maintained in GM-CSF-containing medium. On at least day 7, BM-derived DC's were used experimentally.

Tansgenic (Tg) T cell proliferation: For in vitro proliferation of transgenic T cells, spleens are isolated from naïve TcR Tg mice and CD4₊ T cells are purified using BD Biomag magnetic beads. The purity of T cell isolation is verified by FACS analysis. 1×10⁵ Smarta T cells were cultured in a 96-well plate together with 300-30,000 immature or mature bone-marrow derived dendritic cells. Prior to co-culture, BM-derived DCs were pulsed with 1 μg/ml SMARTA p11 peptide (GPDIYKGVYQFKSVEFD (SEQ ID NO: 12)) (GenScript) in RPMI for 3 hours, followed by washing and irradiation with 2000 rads. Non-pulsed DCs were used as a control as well as T cells cultured alone. Cells were incubated for 4 days and ³-[H]-thymidine was added for the last 18 hours of culture.

No significant difference in T cell priming was observed even when immature DC's were used to activate SMARTA T cells.

Even though the above data imply that there is no deficiency in the ability of IL-18Rα_{-/-} DC's and T cells to become activated, we decided to confirm the activation status of both cell types at the level of activation marker expression. We looked at expression markers on LPS matured DC's as well as KLH-restimulated T cells by FACS, which showed that there is no difference in upregulation of CD80, CD86 and CD40 on IL-18Rα_{-/-} DC's and also no difference in CD5, CD62L and CD44 expression by IL-18Rα_{-/-} T cells, in comparison to wt and IL-18_{-/-} cells (FIG. 6). Therefore the IL-18Rα lesion does not affect T cell or DC activation, at least not at the level of upregulation of surface markers required for adequate stimulation.

### Example 5

### IL-18Rα_{-/-} CD4₊ T Cells Invade the CNS During EAE

EAE is characterized by a massive influx of inflammatory cells into the CNS at the peak of disease yet immune cells also invade the CNS prior to the onset of clinical symptoms (Hickey, W. F. Brain Pathol. 1, 97-105 (1991), Wekerle, H., et al., J. Exp. Biol. 132, 43-57 (1987)). For example, recruitment of CD4₊ T cells into the CNS is critical for the initiation of the effector phase of EAE yet the infiltration of polymorphonuclear leukocytes into the CNS appears to have a role in orchestrating these events (McColl, S. R. et al., J. Immunol. 161, 6421-6426 (1998)). Therefore in order to establish whether IL-18Rα_{-/-} inflammatory cells are completely absent from the CNS at time-points of pre-clinical disease, we immunized mice and analyzed the CNS for inflammatory infiltrates on days 5, 7 and 9 post-immunization.

In contrast to the lack of immune cells at the end-point of disease in IL-18Rα_{-/-} mice (FIG. 2a-e), IL-18Rα_{-/-} CD4₊ T cells were capable of CNS infiltration to the same extent as those of wt and IL-18_{-/-} mice on days 5, 7 and 9 post-immunization, as analyzed by flow cytometry (FIG. 7). There were also comparable numbers of granulocytes, macrophages and B cells present in the CNS. However, as seen in FIG. 2, there is a significant difference in the presence of IL-18Rα_{-/-} inflammatory cells in the CNS at timepoints of clinical disease thus demonstrating their inability to persist during the effector phase of EAE. Interestingly, these results reflect data obtained in IL-23p19_{-/-} mice, which are also resistant to MOG₃₅₋₅₅-induced EAE and in which the deficiency does not prevent infiltration of inflammatory cells into the CNS, as observed on day 7 post-immunization (Langrish, C. L. et al., J. Exp. Med. 201, 233-240 (2005)).

### Example 6

### Lack of IL-18Rα Prevents IL-17 Production

The similarities between IL-18Rα_{-/-} and IL-23_{-/-} mice with regards their EAE resistance with concomitant inflammatory cell invasion into the CNS, provoked us to assess the impact of IL-18Rα on IL-17 production in our mice. IL-17 producing T_{H} cells (T_{H}IL-17) are now accepted to be the main pathogenic population during autoimmune inflammation. To define differences between EAE-susceptible IL-18_{-/-} and EAE-resistant IL-18Rα_{-/-} mice with regards to cytokine secretion, we used a cytokine-protein array (Raybiotech) allowing the simultaneous analysis of 62 different cytokines secreted by lymphocytes upon encountering their cognate recall Ag.

wt. IL-18_{-/-} and IL-18Rα_{-/-} mice were immunized with KLH and 7 days later, lymphocytes were isolated and restimulated with 50 μg/ml KLH (see FIG. 8).

In comparison to IL-18_{-/-} lymphocytes, IL-18Rα_{-/-} lymphocytes produced much less IL-17. To confirm this finding, we analyzed the levels of this cytokine at both the RNA and protein level. Real-time PCR of RNA taken from lymphocytes upon restimulation with KLH showed that the expression of both IL-17 mRNA is significantly decreased in the IL-18Rα_{-/-} cells in comparison to wt and IL-18_{-/-} cells (FIG. 8a). These findings were corroborated by IL-17 ELISA using the supernatant of the same KLH restimulated cells (FIG. 8b).

### Example 7

### The IL-18Rα Lesion Affects Cells in the Accessory Cell Immune Compartment

The lack of IL-18Rα completely prevents the development of EAE via the prevention of T_{H}IL-17 development, whereas its putative ligand IL-18 appears to be irrelevant.

The cell type on which the IL-18Rα exerts its primary effects remains unknown. This is mainly due to the fact that IL-18Rs are expressed by various cell types and tissues. However, one is likely to presume that the presence of IL-18Rα on CD4₊ T cells is absolutely critical for the subsequent polarization of T_{H}IL-17 cells. In order to identify the cell and tissue location of the IL-18Rα lesion in EAE, we selectively expressed IL-18Rα on cells in the leukocyte compartment using irradiation bone-marrow (BM)-chimeras.

### Irradiation Bone Marrow (BM)-Chimeric Mice:

BM-donor mice were euthanized using CO2 and BM-cells were isolated by flushing femur, tibia, radius and hip bones with phosphate buffered solution (PBS). BM cells are then passed through a 100 μm cell strainer and cells are washed with PBS. Recipient mice are lethally irradiated with 1100 rads (split dose) and i.v. injected with 12-25×10⁶ BM-cells. Engraftment takes place over 8 weeks of recovery.

Following irradiation and reconstitution, the APC compartment in secondary lymphoid tissues of recipient mice is comprised entirely of BM cells derived from donor mice (Becher, B., et al., J. Exp. Med. 193, 967-974 (2001)).

We generated BM chimeras by transferring either a 4:1 ratio of RAG_{-/-} and IL-18Rα_{-/-} BM into wt recipients (RAG_{-/-}+IL-18Rα_{-/-}→wt) or IL-18Rα_{-/-} BM only into wt recipients (IL-18Rα−/−→wt). wt-BM was transferred into wt recipients as a control (wt→wt) (Table 2).

RAG_{-/-} mice do not have lymphocytes and the resulting chimera (RAG_{-/-}+IL-18Rα_{-/-}→wt) thus has an IL-18Rα-deficient lymphocyte compartment, whereas the majority of all other leukocytes has undisrupted IL-18Rα alleles.

As expected IL-18Rα_{-/-}→wt mice were resistant to EAE upon immunization with MOG peptide. Alternatively, addition of BM from RAG_{-/-} mice, which has no T or B cells and therefore expresses IL-18Rα only on accessory cells, not on lymphocytes, was able to overcome the resistance of IL-18Rα_{-/-} mice to EAE (FIG. 9). Thus IL-18Rα must exert its primary effects in the accessory cell (mono- and polymorphonucleated phagocytes, DC's & NK-cells) compartment. Again, this finding is highly unexpected, given that IL-18 is thought to exert its effects on T cells and NK cells, but it is completely consistent with our observations so far.

### Example 8

### Lack of IL-18Rα on Host Cells Prevents EAE Development Induced by the Adoptive Transfer of MOG-Reactive T Cells

The above data indicate that the lack of IL-18Rα on accessory cells does not influence T_{H} cell priming and expansion. Furthermore, RAG_{-/-}+IL-18Rα_{-/-}→wt mixed BM-chimeras (FIG. 9) clearly demonstrate that the IL-18Rα deficiency lesions accessory cell function vital for the development of EAE. We subsequently performed an adoptive transfer experiment to reveal the role and function of IL-18R signaling in accessory cells during EAE. To do so, we adoptively transferred encephalitogenic MOG-reactive T cells derived from wt donor mice into groups of both wt and IL-18Rα_{-/-} recipient mice. As expected, fully primed and activated encephalitogenic T cells derived from wt mice induced EAE in wt recipient mice, yet they were incapable of inducing clinical EAE in IL-18Rα-deficient hosts (FIG. 10). This finding again underlines that the IL-18Rα-deficiency lesions a non-lymphocytic leukocyte of the host which is essential for the development of EAE, independent of T cell activity.

### Example 9

### Cloning and Expression of the Soluble IL-18Rα of the Present Invention

DNA constructions allowing the expression of a recombinant antibody, where the variable region of the hIgG1 heavy chain is replaced with the extracellular domain of mouse IL-18Rα and the variable region of the human kappa light chain is replaced either with the extracellular domain of mouse IL-18Rβ, mouse IL-1RacP, mouse IL-1Rrp2, mouse T1/ST2 or mouse IL-1R1 were produced.

The sequences encoding the extracellular domain of mouse IL-18Rα fused to hIgG1 constant heavy chain was cloned in the vector pCEP4 (Invitrogen, cat number V044-50). This vector allows the expression of the extracellular domain of mouse IL-18Rα fused to hIgG1 constant heavy chain. The sequence of said vector is given at SEQ ID NO: 19. The signal peptide of human DEC205 has been used and a 15-amino acid linker sequence consisting of (G₄S)₃ (SEQ ID NO: 15) is encoded between the two parts of the fusion protein.

The sequences encoding the extracellular domain of mouse IL-18Rβ, mouse IL-1RacP, mouse IL-1Rrp2, mouse T1/ST2 or mouse IL-IR1 fused to the constant region of the human kappa light chain was cloned in the vector pCEP4 (Invitrogen, cat number V044-50). The sequence of said vectors which allow the expression of the extracellular domain of mouse IL-18Rβ, mouse IL-1RacP, mouse IL-1Rrp2, mouse T1/ST2 or mouse IL-1R1 fused to the constant region of the human kappa light chain are given at SEQ ID NO: 20, SEQ ID NO: 21, SEQ ID NO: 22, SEQ ID NO: 23 and SEQ ID NO: 24 respectively. The signal peptide of human DEC205 has been used and a 15-amino acid linker sequence consisting of (G₄S)₃ (SEQ ID NO: 15) is encoded between the two parts of the fusion protein.

The below table gives the GenBank accession number of the sequence encoding mouse IL-18Rα, mouse IL-18Rβ, mouse IL-1RacP, mouse IL-1Rrp2, mouse T1/ST2 and mouse IL-1R1 as well as the amino acid (AA) corresponding to the extracellular domain at the protein level.

| Gene | GenBank | Extracellular domain |
|---|---|---|
| IL-18Rα | U43673 | AA21-326 |
| IL-18Rβ | AF077347 | AA15-356 |
| IL-1RAcP | NM_008364 | AA21-359 |
| IL-1Rrp2 | NM_133193 | AA22-340 |
| T1/ST2 | NM_001025602 | AA27-333 |
| IL-1R1 | NM_008362 | AA21-340 |

The recombinant antibody, where the variable region of the hIgG1 heavy chain was replaced with the extracellular domain of mouse IL-18Rα extracellular domain and the variable region of the human kappa light chain was replaced with the extracellular domain of mouse IL-18Rβ was produced (using the technique described for example by Wardemann H et al. (Science, 2003, vol. 301(5638): p1374-7)).

This recombinant antibody (named “catcher αβ”) was expressed in 293 cells and purified over a protein A column using an äkta prime.

The other catcher molecules (soluble receptor of IL-18Rα associated with AcP, IL-1Rrp2, T1/ST2 or IL-IR1 as described herein) can be produced using a similar technology.

The activity of the recombinant antibody (catcher up) was tested for its interfering activity with IL-18 signaling in vitro (see FIG. 12). In this assay, wild type mouse splenocytes were cultured for 24 h in RPMI complete medium plus the indicated cytokines and antibodies. IFNγ secretion was detected by ELISA (following the manufacturers instruction, BD Biosciences). AB stands for a commercially available monoclonal anti-IL-18Rα antibody (clone 112624) (R&D Systems), rat IgG is an isotypic control antibody and catcher up. The result of this experiment provide very clear evidence for the functionality of catcher up, which significantly reduces the production of INFγ at very low concentrations already, suggesting that it has a high affinity for IL-18.

### Example 10

### Biological Activity of the Soluble IL-18Rα of the Present Invention

The biological activity of the soluble receptors of the present invention can be verified using the assay described in example 3.

Briefly, IL-18_{-/-} mice are immunized subcutaneously with MOG₃₅₋₅₅ peptide emulsified in CFA. Mice receive 200 ng pertussis toxin intraperitoneally at the time of immunization and 48 hours later. The soluble IL-18Rα to be tested is administered either 1 day pre-immunization and every 3 days thereafter or every 3 days beginning from disease onset.

Mice are scored daily as follows: 0) no detectable signs of EAE; 0.5) distal tail limp; 1) complete tail limp; 2) unilateral partial hind limb paralysis; 2.5) bilateral partial limb paralysis; 3) complete bilateral hind limb paralysis; 3.5) complete hind limb paralysis and unilateral forelimb paralysis; 4) total paralysis of fore and hind limbs (score >4 to be euthanized); 5) death.

Each time point shown is the average disease score of each group. Statistical significance is assessed using an unpaired Student's t-Test.

TABLE 1
IL-18R is critical for the development of active EAE in mice

| Mouse | | Mean day of | Mean maximal |
|---|---|---|---|
| genotypes | Incidence (%) | disease onset | clinical score (+/− SEM)* |
| Wt | 17/20 (85) | 11.8 | 2.6 +/− 0.13 |
| IL-18−/− | 20/22 (91) | 12.8 | 2.35 +/− 0.13 |
| IL-18R−/− | 2/20 (10) | 18.5 | 2.6 +/− 0.12 |

*of diseased animals

TABLE 2

| Donor bone-marrow | Recipient Mouse | IL-18R Deficiency |
|---|---|---|
| Wt | Wt | No lesion |
| IL-18R−/− | Wt | All cells |
| RAG−/− + IL-18R−/− (1:4) | Wt | Lymphocytes |

1-28. (canceled)

29. An isolated soluble receptor comprising:
a) all or part of the extracellular domain of IL-18Rα or a variant thereof;
b) all or part of the extracellular domain of human IL-18Rα or a variant thereof;
c) amino acid residues 19-132 of SEQ ID NO: 2 or a variant thereof;
d) amino acid residues 122-219 of SEQ ID NO: 2 or a variant thereof;
e) amino acid residues 213-329 of SEQ ID NO: 2 or a variant thereof;
f) amino acid residues 19-219 of SEQ ID NO: 2 or a variant thereof;
g) amino acid residues 122-329 of SEQ ID NO: 2 or a variant thereof;
h) amino acid residues 19-132 and 213-329 of SEQ ID NO: 2 linked by a peptide bond or a variant thereof; or
i) amino acid residues 19-329 of SEQ ID NO: 2 or a variant thereof; and/or a variant of said amino acid residues.

30. The soluble receptor according to claim 29, wherein said variant is a polypeptide having at least 80% identity with said amino acid residues or said IL-18Rα.

31. The soluble receptor according to claim 29, wherein said soluble receptor comprises at least two subunits consisting of amino acid residues 19-132 of SEQ ID NO: 2, and/or amino acid residues 122-219 of SEQ ID NO: 2, and/or amino acid residues 213-329 of SEQ ID NO: 2, and/or amino acid residues 19-219 of SEQ ID NO: 2, and/or 122-329 of SEQ ID NO: 2, and/or amino acid residues 19-132 and 213-329 of SEQ ID NO: 2 linked by a peptide bond, and/or amino acid residues 19-329 of SEQ ID NO: 2, and/or a variant of said amino acid residues, on the same protein backbone as a fusion protein.

32. The soluble receptor according to claim 29, wherein said variant of said amino acid residues is a polypeptide having at least 80% identity with said amino acid residues.

33. The soluble receptor according to claim 32, wherein at least two subunits are the same.

34. The soluble receptor according to claim 29, wherein said soluble receptor is operably linked to an additional amino acid domain.

35. The soluble receptor according to claim 29, further comprising at least one IL-18Rβ subunit that comprises all or part of the extracellular domain of IL-18Rβ.

36. The soluble receptor according to claim 29, further comprising at least one IL-1RacP subunit that comprises all or part of the extracellular domain of IL-1RacP.

37. The soluble receptor according to claim 29, further comprising at least one IL-IR-rp2 subunit that comprises all or part of the extracellular domain of IL-1R-rp2.

38. The soluble receptor according to claim 29, further comprising at least one T1/ST2 subunit that comprises all or part of the extracellular domain of T1/ST2.

39. The soluble receptor according to claim 29, further comprising at least one IL-1R-1 subunit that comprises all or part of the extracellular domain of IL-1R-1.

40. A multimer comprising a soluble receptor according to claim 29.

41. A method of treating or ameliorating the symptoms of an autoimmune or demyelinating disease in a subject, said method comprising administering to the subject a therapeutically effective amount of a soluble receptor according to claim 29.

42. A method according to claim 41 wherein the subject is human.

43. The method according to claim 41, wherein said demyelinating disease is multiple sclerosis (MS).

44. The method according to claim 41, wherein the subject is affected by relapsing-remitting (RR) multiple sclerosis, secondary progressive (SP) multiple sclerosis, primary progressive (PP) multiple sclerosis or progressive relapsing (PR) multiple sclerosis.

45. The method according to claim 41, wherein the soluble receptor is administered in conjunction with a second therapeutic agent for treating MS.

46. The method according to claim 41, wherein the soluble receptor is administered in conjunction with corticosteroids, immunosuppressive drugs, neuro-protective agents, immunomodulatory drugs or interferons.

47. The method according to claim 41, wherein the soluble receptor is administered in conjunction with interferon-beta or interferon beta-1a.

48. A composition comprising a soluble receptor according to claim 29 and a corticosteroid, an immunosuppressive drug, a neuro-protective agent, an immunomodulatory drug or an interferon.

49. The composition according to claim 48, wherein the interferon is interferon-beta or interferon beta-1a.

50. A composition comprising a soluble receptor according to claim 29 and pharmaceutically acceptable diluents, carriers, biologically compatible vehicles or additives.
